(19) United States
(12) Patent Application Publication
(10) Pub. No.: US 2023/0365995 A1
(43) Pub. Date: Nov. 16, 2023

(54) LIPID NANOPARTICLE COMPOSITIONS

(71) Applicant: Precision BioSciences, Inc., Durham, NC (US)

(72) Inventor: Tyler Goodwin, Durham, NC (US)

(73) Assignee: Precision BioSciences, Inc., Durham, NC (US)

(21) Appl. No.: 18/248,146

(22) PCT Filed: Oct. 6, 2021
(86) PCT No.: PCT/US2021/053757

§ 371 Date: Apr. 6, 2023

Related U.S. Application Data
(60) Provisional application No. 63/088,816, filed on Oct. 7, 2020.

(51) Int. Cl. C12N 15/88; A61K 47/69; A61K 47/62; A61K 9/51; A61K 47/60

(52) U.S. Cl. CPC C12N 15/88; A61K 47/6929; A61K 47/62; A61K 9/5123; A61K 9/5169; A61K 47/60

(57) ABSTRACT

The present invention provides lipid nanoparticle compositions, and methods of using the same, that are useful for introducing nucleic acids into eukaryotic cells, such as human immune cells. Generally, the lipid nanoparticles described herein comprise an apolipoprotein that is bound to one component of the lipid nanoparticles. The apolipoprotein can be bound to the lipid nanoparticle components by various types of bonds, including covalent bonds. The invention further provides methods for transfecting eukaryotic cells with such lipid nanoparticles, populations of eukaryotic cells, pharmaceutical compositions, and methods of treatment and use.

### FIELD OF THE INVENTION

The present invention generally relates to the field of lipid nanoparticle technology. In particular, the invention relates to a simplified method for introducing nucleic acids into eukaryotic cells, such as human immune cells.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Oct. 6, 2021, is named P109070052WO00-SEQ-MJT, and is 5,187 bytes in size.

### BACKGROUND OF THE INVENTION

Genetic modification of human immune cells is being leveraged for a number of therapeutic approaches, including the development of T cells and natural killer (NK) cells expressing chimeric antigen receptors (CARS) or exogenous T cell receptors (TCRs). Such cells induce tumor immunoreactivity in a major histocompatibility complex non-restricted manner. Adoptive immunotherapy has been utilized as a clinical therapy for a number of cancers, including B cell malignancies (e.g., acute lymphoblastic leukemia, B cell non-Hodgkin lymphoma, acute myeloid leukemia, and chronic lymphocytic leukemia), multiple myeloma, neuroblastoma, glioblastoma, advanced gliomas, ovarian cancer, mesothelioma, melanoma, prostate cancer, pancreatic cancer, and others.

Typically, a coding sequence for a CAR or TCR is introduced into the cell by a viral vector. In some cases, the coding sequence is randomly integrated into the genome of the cell using a lentiviral vector. Insertion of the CAR or TCR coding sequence can be accompanied by the use of an engineered nuclease to knock out certain genes of interest. For example, T cells expressing an endogenous T cell receptor may recognize major and minor histocompatibility antigens following administration to an allogeneic patient, which can lead to the development of graft-versus-host-disease (GVHD). To avoid this outcome, an engineered nuclease can be used to knock out expression of an endogenous TCR (e.g., an alpha/beta TCR) in order to produce to a T cell useful for allogeneic administration.

In other cases, the coding sequence for a CAR or exogenous TCR is specifically inserted in a target gene. Generally, the process of targeted insertion is made possible by the use of an engineered nuclease which generates a double-stranded cleavage site in the genome at the target gene. The CAR or TCR coding sequence is then inserted at the cleavage site by homologous recombination of a donor template, resulting in expression of the transgene while disrupting expression of the protein encoded by the target gene.

Engineered nucleases are usually introduced into immune cells (e.g., T cells and NK cells) using mRNA, typically by the process of electroporation. This method exposes the cells to a number of electrical and mechanical stresses that impact cell viability, number, and proliferation in the aftermath of the process. Furthermore, when a donor template comprising a CAR or TCR coding sequence is also introduced, this is often done by contacting the cells with an adeno-associated virus (AAV) comprising the donor template. Methods that include both the introduction of a nucleic acid encoding a nuclease, and the introduction of a CAR or TCR coding sequence, often require a number of centrifugation, buffer change, and vessel transfer steps that further impact recovery and performance of the cell population.

It is well established that primary eukaryotic cells, such as primary immune cells, are notoriously difficult to transfect with nucleic acids, making it difficult to directly introduce nucleic acids without the use of electroporation or viruses. Accordingly, there remains a need in the art for additional methods of transfection that allow for simplified introduction of nucleic acids into eukaryotic cells, such as primary immune cells, without producing the negative effects associated with current methods.

### SUMMARY OF THE INVENTION

The present invention provides lipid nanoparticle compositions, and methods of using the same, that are useful for introducing nucleic acids into eukaryotic cells (e.g., T cells and NK cells). Generally, the lipid nanoparticles described herein comprise an apolipoprotein (e.g., ApoE) that is bound to one component of the lipid nanoparticles, such as a cationic lipid, a non-cationic lipid (e.g., a steroid or a phospholipid), or a lipid conjugate. The apolipoprotein can be bound to the lipid nanoparticle components by various types of bonds. For example, the apolipoprotein can be bound by a covalent bond to a lipid that has been chemically modified to enable such a bond. It has been discovered that lipid nanoparticles comprising a bound apolipoprotein can facilitate binding of the lipid nanoparticle to eukaryotic cells comprising cell surface low density lipoprotein (LDL) receptors, enhancing delivery of encapsulated nucleic acids into the cells. In particular examples, such lipid nanoparticles allow for efficient uptake and expression of mRNA or DNA encoding an engineered nuclease, subsequent gene editing and disruption, and/or targeted insertion of a donor template encoding a polypeptide of interest (e.g., a CAR or exogenous TCR) at the nuclease cleavage site. In other particular examples, the lipid nanoparticles described herein can be used to deliver a donor template into a eukaryotic cell that is inserted into the genome at a nuclease cleavage site.

Thus, in one aspect, the invention provides a lipid nanoparticle composition comprising lipid nanoparticles comprising: (a) one or more cationic lipids; (b) one or more non-cationic lipids; (c) one or more lipid conjugates; and (d) an apolipoprotein bound to at least one of the one or more cationic lipids, at least one of the one or more non-cationic lipids, or at least one of the one or more lipid conjugates.

In some embodiments, the apolipoprotein is bound by hydrophobic bonds. In certain embodiments, the apolipoprotein is bound by hydrophilic bonds. In certain embodiments, the apolipoprotein is bound by noncovalent bonds.

In some embodiments, the lipid nanoparticle composition comprises lipid nanoparticles comprising: (a) one or more cationic lipids; (b) one or more non-cationic lipids; (c) one or more lipid conjugates; and (d) an apolipoprotein covalently bound to at least one of the one or more cationic lipids, at least one of the one or more non-cationic lipids, or at least one of the one or more lipid conjugates.

In some embodiments, at least one of the one or more cationic lipids is a chemically modified cationic lipid and bound to the apolipoprotein. In some embodiments, at least one of the one or more non-cationic lipids is a chemically modified non-cationic lipid and bound to the apolipoprotein. In some embodiments, at least one of the one or more lipid conjugates is a chemically modified lipid conjugate and bound to the apolipoprotein. Such chemical modifications are intended to enable the lipids to form a covalent bond with the apolipoprotein.

In certain embodiments, the apolipoprotein is bound to a terminus of at least one of the one or more cationic lipids. In certain embodiments, the apolipoprotein is bound to a terminus of at least one of the one or more non-cationic lipids. In certain embodiments, the apolipoprotein is bound to a terminus of at least one of the one or more lipid conjugates.

In some embodiments, the chemically modified cationic lipid is chemically modified at its terminus, and the apolipoprotein is bound at the terminus. In some embodiments, the chemically modified non-cationic lipid is chemically modified at its terminus, and the apolipoprotein is bound at the terminus. In some embodiments, the chemically modified lipid conjugate is chemically modified at its terminus, and the apolipoprotein is bound at the terminus.

In certain embodiments, the modification of the chemically modified cationic lipid, the chemically modified non-cationic lipid, or the chemically modified lipid conjugate comprises an introduced amino group. In certain embodiments, the modification of the chemically modified cationic lipid, the chemically modified non-cationic lipid, or the chemically modified lipid conjugate comprises an introduced carboxyl group. In certain embodiments, the modification of the chemically modified cationic lipid, the chemically modified non-cationic lipid, or the chemically modified lipid conjugate comprises an introduced hydroxyl group. In certain embodiments, the modification of the chemically modified cationic lipid, the chemically modified non-cationic lipid, or the chemically modified lipid conjugate comprises an introduced sulfhydryl group. In certain embodiments, the modification of the chemically modified cationic lipid, the chemically modified non-cationic lipid, or the chemically modified lipid conjugate comprises an introduced maleimide. In certain embodiments, the modification of the chemically modified cationic lipid, the chemically modified non-cationic lipid, or the chemically modified lipid conjugate comprises an introduced acyl halide group. In certain embodiments, the modification of the chemically modified cationic lipid, the chemically modified non-cationic lipid, or the chemically modified lipid conjugate comprises an introduced acetyl halide group. In certain embodiments, the modification of the chemically modified cationic lipid, the chemically modified non-cationic lipid, or the chemically modified lipid conjugate comprises an introduced aldehyde group. In certain embodiments, the modification of the chemically modified cationic lipid, the chemically modified non-cationic lipid, or the chemically modified lipid conjugate comprises an introduced azo group. In certain embodiments, the modification of the chemically modified cationic lipid, the chemically modified non-cationic lipid, or the chemically modified lipid conjugate comprises an introduced azide group. In certain embodiments, the modification of the chemically modified cationic lipid, the chemically modified non-cationic lipid, or the chemically modified lipid conjugate comprises an introduced alkyne group. In certain embodiments, the modification of the chemically modified cationic lipid, the chemically modified non-cationic lipid, or the chemically modified lipid conjugate comprises an introduced alkene group. In certain embodiments, the modification of the chemically modified cationic lipid, the chemically modified non-cationic lipid, or the chemically modified lipid conjugate comprises an introduced haloalkane group. In certain embodiments, the modification of the chemically modified cationic lipid, the chemically modified non-cationic lipid, or the chemically modified lipid conjugate comprises an introduced phosphine group. In certain embodiments, the modification of the chemically modified cationic lipid, the chemically modified non-cationic lipid, or the chemically modified lipid conjugate comprises an introduced imine group. In certain embodiments, the modification of the chemically modified cationic lipid, the chemically modified non-cationic lipid, or the chemically modified lipid conjugate comprises an introduced thiol group. In certain embodiments, the modification of the chemically modified cationic lipid, the chemically modified non-cationic lipid, or the chemically modified lipid conjugate comprises an introduced sulfoxide group. In certain embodiments, the modification of the chemically modified cationic lipid, the chemically modified non-cationic lipid, or the chemically modified lipid conjugate comprises an introduced sulfone group. In certain embodiments, the modification of the chemically modified cationic lipid, the chemically modified non-cationic lipid, or the chemically modified lipid conjugate comprises an introduced sulfonic acid group. In certain embodiments, the modification of the chemically modified cationic lipid, the chemically modified non-cationic lipid, or the chemically modified lipid conjugate comprises an introduced sulfide group. In certain embodiments, the modification of the chemically modified cationic lipid, the chemically modified non-cationic lipid, or the chemically modified lipid conjugate comprises an introduced peroxide group. In certain embodiments, the modification of the chemically modified cationic lipid, the chemically modified non-cationic lipid, or the chemically modified lipid conjugate comprises an introduced chelating group. In certain embodiments, the modification of the chemically modified cationic lipid, the chemically modified non-cationic lipid, or the chemically modified lipid conjugate comprises an introduced ester group. In certain embodiments, the modification of the chemically modified cationic lipid, the chemically modified non-cationic lipid, or the chemically modified lipid conjugate comprises an introduced epoxide group. In certain embodiments, the modification of the chemically modified cationic lipid, the chemically modified non-cationic lipid, or the chemically modified lipid conjugate comprises an introduced nitrone group. In certain embodiments, the modification of the chemically modified cationic lipid, the chemically modified non-cationic lipid, or the chemically modified lipid conjugate comprises an introduced cyclooctynes group. In certain embodiments, the modification of the chemically modified cationic lipid, the chemically modified non-cationic lipid, or the chemically modified lipid conjugate comprises an introduced sulfonyl halides group. In certain embodiments, the modification of the chemically modified cationic lipid, the chemically modified non-cationic lipid, or the chemically modified lipid conjugate comprises an introduced β-propiolactone group. In certain embodiments, the modification of the chemically modified cationic lipid, the chemically modified non-cationic lipid, or the chemically modified lipid conjugate comprises an introduced γ-butyrolactone group. In certain embodiments, the modification of the chemically modified cationic lipid, the chemically modified non-cationic lipid, or the chemically modified lipid conjugate comprises an introduced β-lactam group. In certain embodiments, the modification of the chemically modified cationic lipid, the chemically modified non-cationic lipid, or the chemically modified lipid conjugate comprises an introduced boronic acid group. In certain embodiments, the modification of the chemically modified cationic lipid, the chemically modified non-cationic lipid, or the chemically modified lipid conjugate comprises an introduced aryl urea with one nitrogen being in aliphatic ring group.

In some embodiments, the apolipoprotein is bound by a covalent bond. In certain embodiments, the apolipoprotein is covalently bound by an amide bond. In certain embodiments, the apolipoprotein is covalently bound by a thioester bond. In some embodiments, the apolipoprotein is covalently bound by a disulfide bond. In some embodiments, the apolipoprotein is covalently bound by a hydrazone bond. In some embodiments, the apolipoprotein is covalently bound by an imine bond. In some embodiments, the apolipoprotein is covalently bound by an azole bond. In some embodiments, the apolipoprotein is covalently bound by a triazole bond.

In certain embodiments, the one or more cationic lipids includes at least one chemically modified cationic lipid and at least one unmodified cationic lipid. In some embodiments, the chemically modified cationic lipid is derived from the unmodified cationic lipid. In some embodiments, the chemically modified cationic lipid is not derived from the unmodified cationic lipid.

In some embodiments, the one or more non-cationic lipids includes at least one chemically modified non-cationic lipid and at least one unmodified non-cationic lipid. In some embodiments, the chemically modified non-cationic lipid is derived from the unmodified non-cationic lipid. In some embodiments, the chemically modified cationic lipid is not derived from the unmodified non-cationic lipid.

In certain embodiments, the one or more lipid conjugates includes at least one chemically modified lipid conjugate and at least one unmodified lipid conjugate. In some embodiments, the chemically modified lipid conjugate is derived from the unmodified lipid conjugate. In some embodiments, the chemically modified lipid conjugate is not derived from the unmodified lipid conjugate.

In some embodiments, the apolipoprotein is an apolipoprotein A (ApoA). In some embodiments, the apolipoprotein is an apolipoprotein B (ApoB). In some embodiments, the apolipoprotein is an apolipoprotein C (ApoC). In some embodiments, the apolipoprotein is an apolipoprotein D (ApoD). In some embodiments, the apolipoprotein is an apolipoprotein E (ApoE). In some embodiments, the apolipoprotein is an apolipoprotein H (ApoH). In some embodiments, the apolipoprotein is an apolipoprotein L (ApoL). In some embodiments, the apolipoprotein is an apolipoprotein M (ApoM). In some embodiments, the apolipoprotein is an or apolipoprotein (a) (Apo(a)).

In particular embodiments, apolipoprotein is ApoE. In certain embodiments, the apolipoprotein is ApoE isoform 2. In certain embodiments, the apolipoprotein is ApoE isoform 3. In certain embodiments, the apolipoprotein is ApoE isoform 4.

In some embodiments, the total molar concentration of the one or more cationic lipids is from about 20% to about 80% of the total lipid molar concentration. In some embodiments, the total molar concentration of the one or more cationic lipids is from about 30% to about 70% of the total lipid molar concentration. In some embodiments, the total molar concentration of the one or more cationic lipids is from about 40% to about 60% of the total lipid molar concentration. In some embodiments, the total molar concentration of the one or more cationic lipids is from about 45% to about 55% of the total lipid molar concentration. In certain embodiments, the total molar concentration of the one or more cationic lipids is about 20%, about 20.5%, about 21%, about 21.5%, about 22%, about 22.5%, about 23%, about 23.5%, about 24%, about 24.5%, about 25%, about 25.5%, about 26%, about 26.5%, about 27%, about 27.5%, about 28%, about 28.5%, about 29%, about 29.5%, about 30%, about 30.5%, about 31%, about 31.5%, about 32%, about 32.5%, about 33%, about 33.5%, about 34%, about 34.5%, about 35%, about 35.5%, about 36%, about 36.5%, about 37%, about 37.5%, about 38%, about 38.5%, about 39%, about 39.5%, about 40%, about 40.5%, about 41%, about 41.5%, about 42%, about 42.5%, about 43%, about 43.5%, about 44%, about 44.5%, about 45%, about 45.5%, about 46%, about 46.5%, about 47%, about 47.5%, about 48%, about 48.5%, about 49%, about 49.5%, about 50%, about 50.5%, about 51%, about 51.5%, about 52%, about 52.5%, about 53%, about 53.5%, about 54%, about 54.5%, about 55%, about 55.5%, about 56%, about 56.5%, about 57%, about 57.5%, about 58%, about 58.5%, about 59%, about 59.5%, about 60%, about 60.5%, about 61%, about 61.5%, about 62%, about 62.5%, about 63%, about 63.5%, about 64%, about 64.5%, about 65%, about 65.5%, about 66%, about 66.5%, about 67%, about 67.5%, about 68%, about 68.5%, about 69%, about 69.5%, about 70%, about 70.5%, about 71%, about 71.5%, about 72%, about 72.5%, about 73%, about 73.5%, about 74%, about 74.5%, about 75%, about 75.5%, about 76%, about 76.5%, about 77%, or about 77.5% of the total lipid molar concentration.

In some embodiments, the total molar concentration of the one or more non-cationic lipids is from about 20% to about 80% of the total lipid molar concentration. In some embodiments, the total molar concentration of the one or more non-cationic lipids is from about 30% to about 70% of the total lipid molar concentration. In some embodiments, the total molar concentration of the one or more non-cationic lipids is from about 40% to about 70% of the total lipid molar concentration. In some embodiments, the total molar concentration of the one or more non-cationic lipids is from about 40% to about 60% of the total lipid molar concentration. In some embodiments, the total molar concentration of the one or more non-cationic lipids is from about 45% to about 55% of the total lipid molar concentration. In certain embodiments, the total molar concentration of the one or more non-cationic lipids is about 20%, about 20.5%, about 21%, about 21.5%, about 22%, about 22.4%, about 22.5%, about 22.9%, about 23%, about 23.4%, about 23.5%, about 23.9%, about 24%, about 24.4%, about 24.5%, about 24.9%, about 25%, about 25.4%, about 25.5%, about 25.9%, about 26%, about 26.4%, about 26.5%, about 26.9%, about 27%, about 27.4%, about 27.5%, about 27.9%, about 28%, about 28.4%, about 28.5%, about 28.9%, about 29%, about 29.4%, about 29.5%, about 29.9%, about 30%, about 30.4%, about 30.5%, about 30.9%, about 31%, about 31.4%, about 31.5%, about 31.9%, about 32%, about 32.4%, about 32.5%, about 32.9%, about 33%, about 33.4%, about 33.5%, about 33.9%, about 34%, about 34.4%, about 34.5%, about 34.9%, about 35%, about 35.4%, about 35.5%, about 35.9%, about 36%, about 36.4%, about 36.5%, about 36.9%, about 37%, about 37.4%, about 37.5%, about 37.9%, about 38%, about 38.4%, about 38.5%, about 38.9%, about 39%, about 39.4%, about 39.5%, about 39.9%, about 40%, about 40.4%, about 40.4%, about 40.5%, about 40.9%, about 41%, about 41.4%, about 41.5%, about 41.9%, about 42%, about 42.4%, about 42.5%, about 42.9%, about 43%, about 43.4%, about 43.5%, about 43.9%, about 44%, about 44.4%, about 44.5%, about 44.9%, about 45%, about 45.4%, about 45.5%, about 45.9%, about 46%, about 46.4%, about 46.5%, about 46.9%, about 47%, about 47.4%, about 47.5%, about 47.9%, about 48%, about 48.4%, about 48.5%, about 48.9%, about 49%, about 49.4%, about 49.5%, about 49.9%, about 50%, about 50.4%, about 50.5%, about 50.9%, about 51%, about 51.4%, about 51.5%, about 51.9%, about 52%, about 52.4%, about 52.5%, about 52.9%, about 53%, about 53.4%, about 53.5%, about 53.9%, about 54%, about 54.4%, about 54.5%, about 54.9%, about 55%, about 55.4%, about 55.5%, about 55.9%, about 56%, about 56.4%, about 56.5%, about 56.9%, about 57%, about 57.4%, about 57.5%, about 57.9%, about 58%, about 58.4%, about 58.5%, about 58.9%, about 59%, about 59.4%, about 59.5%, about 59.9%, about 60%, about 60.4%, about 60.5%, about 60.9%, about 61%, about 61.4%, about 61.5%, about 61.9%, about 62%, about 62.4%, about 62.5%, about 62.9%, about 63%, about 63.4%, about 63.5%, about 63.9%, about 64%, about 64.4%, about 64.5%, about 64.9%, about 65%, about 65.4%, about 65.5%, about 65.9%, about 66%, about 66.4%, about 66.5%, about 66.9%, about 67%, about 67.4%, about 67.5%, about 67.9%, about 68%, about 68.4%, about 68.5%, about 68.9%, about 69%, about 69.4%, about 69.5%, about 69.9%, about 70%, about 70.4%, about 70.5%, about 70.9%, about 71%, about 71.4%, about 71.5%, about 71.9%, about 72%, about 72.4%, about 72.5%, about 72.9%, about 73%, about 73.4%, about 73.5%, about 73.9%, about 74%, about 74.4%, about 74.5%, about 74.9%, about 75%, about 75.4%, about 75.5%, about 75.9%, about 76%, about 76.4%, about 76.5%, about 76.9%, about 77%, about 77.4%, about 77.5%, about 77.9%, about 78%, about 78.4%, about 78.5%, about 78.9%, about 79%, about 79.4%, about 79.5%, or about 79.9% of the total lipid molar concentration.

In some embodiments, the one or more non-cationic lipids includes one or more phospholipids. In certain embodiments, the total molar concentration of the one or more phospholipids is from about 0% to about 30% of the total lipid molar concentration. In certain embodiments, the total molar concentration of the one or more phospholipids is from about 2.5% to about 25% of the total lipid total molar concentration. In certain embodiments, the total molar concentration of the one or more phospholipids is from about 5% to about 20% of the total lipid molar concentration. In certain embodiments, the total molar concentration of the one or more phospholipids is from about 5% to about 15% of the total lipid molar concentration. In certain embodiments, the total molar concentration of the one or more phospholipids is from about 7.5% to about 12.5% of the total lipid molar concentration. In certain embodiments, the total molar concentration of the one or more phospholipids is about 5% of the total lipid molar concentration. In certain embodiments, the total molar concentration of the one or more phospholipids is about 7.5% of the total lipid molar concentration. In certain embodiments, the total molar concentration of the one or more phospholipids is about 10% of the total lipid molar concentration. In certain embodiments, the total molar concentration of the one or more phospholipids is about 12.5% of the total lipid molar concentration. In certain embodiments, the total molar concentration of the one or more phospholipids is about 15% of the total lipid molar concentration. In certain embodiments, the total molar concentration of the one or more phospholipids is about 17.5% of the total lipid molar concentration. In certain embodiments, the total molar concentration of the one or more phospholipids is about 20% of the total lipid molar concentration.

In some embodiments, the one or more non-cationic lipids includes one or more steroids. In certain embodiments, the total molar concentration of the one or more steroids is from about 20% to about 60% of the total lipid molar concentration. In certain embodiments, the total molar concentration of the one or more steroids is from about 25% to about 55% of the total lipid molar concentration. In certain embodiments, the total molar concentration of the one or more steroids is from about 30% to about 50% of the total lipid molar concentration. In certain embodiments, the total molar concentration of the one or more steroids is from about 35% to about 40% of the total lipid molar concentration. In certain embodiments, the total molar concentration of the one or more steroids is about 15%, about 15.5%, about 16%, about 16.5%, about 17%, about 17.4%, about 17.5%, about 17.9%, about 18%, about 18.4%, about 18.5%, about 18.9%, about 19%, about 19.4%, about 19.5%, about 19.9%, about 20%, about 20.4%, about 20.5%, about 20.9%, about 21%, about 21.4%, about 21.5%, about 21.9%, about 22%, about 22.4%, about 22.5%, about 22.9%, about 23%, about 23.4%, about 23.5%, about 23.9%, about 24%, about 24.4%, about 24.5%, about 24.9%, about 25%, about 25.4%, about 25.5%, about 25.9%, about 26%, about 26.4%, about 26.5%, about 26.9%, about 27%, about 27.4%, about 27.5%, about 27.9%, about 28%, about 28.4%, about 28.5%, about 28.9%, about 29%, about 29.4%, about 29.5%, about 29.9%, about 30%, about 30.4%, about 30.5%, about 30.9%, about 31%, about 31.4%, about 31.5%, about 31.9%, about 32%, about 32.4%, about 32.5%, about 32.9%, about 33%, about 33.4%, about 33.5%, about 33.9%, about 34%, about 34.4%, about 34.5%, about 34.9%, about 35%, about 35.4%, about 35.5%, about 35.9%, about 36%, about 36.4%, about 36.5%, about 36.9%, about 37%, about 37.4%, about 37.5%, about 37.9%, about 38%, about 38.4%, about 38.5%, about 38.9%, about 39%, about 39.4%, about 39.5%, about 39.9%, about 40%, about 40.4%, about 40.5%, about 40.9%, about 41%, about 41.4%, about 41.5%, about 41.9%, about 42%, about 42.4%, about 42.5%, about 42.9%, about 43%, about 43.4%, about 43.5%, about 43.9%, about 44%, about 44.4%, about 44.5%, about 44.9%, about 45%, about 45.4%, about 45.5%, about 45.9%, about 46%, about 46.4%, about 46.5%, about 46.9%, about 47%, about 47.4%, about 47.5%, about 48%, about 48.4%, about 48.5%, about 48.9%, about 49%, about 49.4%, about 49.5%, about 49.9%, about 50%, about 50.4%, about 50.5%, about 50.9%, about 51%, about 51.4%, about 51.5%, about 51.9%, about 52%, about 52.4%, about 52.5%, about 52.9%, about 53%, about 53.4%, about 53.5%, about 53.9%, about 54%, about 54.4%, about 54.5%, about 54.9%, about 55%, about 55.4%, about 55.5%, about 55.9%, about 56%, about 56.4%, about 56.5%, about 56.9%, about 57%, about 57.4%, about 57.5%, about 579%, about 58%, about 58.4%, about 58.5%, about 58.9%, about 59%, about 59.4%, about 59.5%, about 59.9%, about 60%, about 60.4%, about 60.5%, about 60.9%, about 61%, about 61.4%, about 61.5%, about 61.9%, about 62%, about 62.4%, about 62.5%, about 62.9%, about 63%, about 63.4%, about 63.5%, about 63.9%, about 64%, about 64.4%, about 64.5%, about 64.9%, about 65%, about 65.4%, about 65.5%, about 65.9%, about 66%, about 66.4%, about 66.5%, about 66.9%, about 67%, about 67.4%, about 67.5%, about 67.9%, about 68%, about 68.4%, about 68.5%, about 68.9%, about 69%, about 69.4%, about 69.5%, about 69.9%, about 70%, about 70.4%, about 70.5%, about 70.9%, about 71%, about 71.4%, about 71.5%, about 71.9%, about 72%, about 72.4%, about 72.5%, about 72.9%, about 73%, about 73.4%, about 73.5%, about 73.9%, about 74%, about 74.4%, about 74.5%, about 74.9%, or about 75% of the total lipid molar concentration.

In certain embodiments, the one or more non-cationic lipids comprise a phospholipid and a steroid.

In some embodiments, the total molar concentration of the one or more lipid conjugates is from about 0.01% to about 10% of the total lipid molar concentration. In some embodiments, the total molar concentration of the one or more lipid conjugates is from about 0.1% to about 10% of the total lipid molar concentration. In some embodiments, the total molar concentration of the one or more lipid conjugates is from about 0.2% to about 8% of the total lipid molar concentration. In some embodiments, the total molar concentration of the one or more lipid conjugates is from about 0.5% to about 5% of the total lipid molar concentration. In some embodiments, the total molar concentration of the one or more lipid conjugates is from about 0.1% to about 1.5% of the total lipid molar concentration. In some embodiments, the total molar concentration of the one or more lipid conjugates is from about 1% to about 2% of the total lipid molar concentration. In certain embodiments, the total molar concentration of the one or more lipid conjugates is about 0.1% of the total lipid molar concentration. In certain embodiments, the total molar concentration of the one or more lipid conjugates is about 0.5% of the total lipid molar concentration. In certain embodiments, the total molar concentration of the one or more lipid conjugates is about 1% of the total lipid molar concentration. In certain embodiments, the total molar concentration of the one or more lipid conjugates is about 1.5% of the total lipid molar concentration. In certain embodiments, the total molar concentration of the one or more lipid conjugates is about 2% of the total lipid molar concentration. In certain embodiments, the total molar concentration of the one or more lipid conjugates is about 2.5% of the total lipid molar concentration.

In some embodiments, the lipid nanoparticle comprises the one or more cationic lipids, the one or more phospholipids, the one or more steroids, and the one or more lipid conjugates at a molar ratio set forth in any embodiment of lipid nanoparticle composition disclosed in the table of FIG. 1. In certain embodiments, the lipid nanoparticle comprises the combination of molar percentages as set forth in any one of embodiments, 1-3204 in the table of FIG. 1.

In certain embodiments, the lipid nanoparticles comprise: (a) the one or more cationic lipids at a total molar concentration of about 40% of the total lipid molar concentration; (b) the one or more steroids at a total molar concentration of about 48.5% of the total lipid molar concentration; (c) the one or more phospholipids at a total molar concentration of about 10% of the total lipid molar concentration; and (d) the one or more lipid conjugates at a molar concentration of about 1.5% of the total lipid molar concentration.

In certain embodiments, the lipid nanoparticles comprise: (a) the one or more cationic lipids at a total molar concentration of about 50% of the total lipid molar concentration; (b) the one or more steroids at a total molar concentration of about 38.5% of the total lipid molar concentration; (c) the one or more phospholipids at a total molar concentration of about 10% of the total lipid molar concentration; and (d) the one or more lipid conjugates at a total molar concentration of about 1.5% of the total lipid molar concentration.

In some embodiments, the one or more cationic lipids includes DLin-DMA, or a derivative thereof. In some embodiments, the one or more cationic lipids includes DLin-MC3-DMA, or a derivative thereof. In some embodiments, the one or more cationic lipids includes DLin-KC2-DMA, or a derivative thereof. In some embodiments, the one or more cationic lipids includes DODMA, or a derivative thereof. In some embodiments, the one or more cationic lipids includes SS-OP, or a derivative thereof. In some embodiments, the one or more cationic lipids includes SS-M, or a derivative thereof. In some embodiments, the one or more cationic lipids includes SS-E, or a derivative thereof. In some embodiments, the one or more cationic lipids includes SS-EC, or a derivative thereof. In some embodiments, the one or more cationic lipids includes SS-LC, or a derivative thereof. In some embodiments, the one or more cationic lipids includes SS-OC, or a derivative thereof. In some embodiments, the one or more cationic lipids includes DOTAP, or a derivative thereof. In some embodiments, the one or more cationic lipids includes DOTMA, or a derivative thereof. In some embodiments, the one or more cationic lipids includes DODAP, or a derivative thereof. In some embodiments, the one or more cationic lipids includes DOGS, or a derivative thereof. In some embodiments, the one or more cationic lipids includes DOSPA, or a derivative thereof. In some embodiments, the one or more cationic lipids includes DC-Chol, or a derivative thereof. In some embodiments, the one or more cationic lipids includes GL-67, or a derivative thereof. In some embodiments, the one or more cationic lipids includes BGTC, or a derivative thereof. In some embodiments, the one or more cationic lipids includes DDAB, or a derivative thereof. In some embodiments, the one or more cationic lipids includes DORIE, or a derivative thereof. In some embodiments, the one or more cationic lipids includes DMRIE, or a derivative thereof. In some embodiments, the one or more cationic lipids includes GAP-DLRIE, or a derivative thereof. In some embodiments, the one or more cationic lipids includes diC14-amidine, or a derivative thereof. In some embodiments, the one or more cationic lipids includes L319, or a derivative thereof. In some embodiments, the one or more cationic lipids includes C12-200, or a derivative thereof. In some embodiments, the one or more cationic lipids includes OF-02, or a derivative thereof. In some embodiments, the one or more cationic lipids includes TT3, or a derivative thereof. In some embodiments, the one or more cationic lipids includes ZA3-Ep10, or a derivative thereof.

In certain embodiments, the one or more cationic lipids includes at least one chemically modified cationic lipid bound to the apolipoprotein, and at least one unmodified cationic lipid. In some embodiments, the molar ratio of the chemically modified cationic lipid to the unmodified cationic lipid is between about 1:1 and about 1:300. In some embodiments, the molar ratio of the chemically modified cationic lipid to the unmodified cationic lipid is between about 1:10 and about 1:200. In some embodiments, the molar ratio of the chemically modified cationic lipid to the unmodified cationic lipid is between about 1:25 and about 1:175. In some embodiments, the molar ratio of the chemically modified cationic lipid to the unmodified cationic lipid is between about 1:50 and about 1:150. In some embodiments, the molar ratio of the chemically modified cationic lipid to the unmodified cationic lipid is between about 1:100 and about 1:150. In some embodiments, the molar ratio of the chemically modified cationic lipid to the unmodified cationic lipid is between about 1:125 and about 1:150. In particular embodiments, the molar ratio of the chemically modified cationic lipid to the unmodified cationic lipid is about 1:50. In particular embodiments, the molar ratio of the chemically modified cationic lipid to the unmodified cationic lipid is about 1:60. In particular embodiments, the molar ratio of the chemically modified cationic lipid to the unmodified cationic lipid is about 1:70. In particular embodiments, the molar ratio of the chemically modified cationic lipid to the unmodified cationic lipid is about 1:80. In particular embodiments, the molar ratio of the chemically modified cationic lipid to the unmodified cationic lipid is about 1:90. In particular embodiments, the molar ratio of the chemically modified cationic lipid to the unmodified cationic lipid is about 1:100. In particular embodiments, the molar ratio of the chemically modified cationic lipid to the unmodified cationic lipid is about 1:105. In particular embodiments, the molar ratio of the chemically modified cationic lipid to the unmodified cationic lipid is about 1:110. In particular embodiments, the molar ratio of the chemically modified cationic lipid to the unmodified cationic lipid is about 1:115. In particular embodiments, the molar ratio of the chemically modified cationic lipid to the unmodified cationic lipid is about 1:120. In particular embodiments, the molar ratio of the chemically modified cationic lipid to the unmodified cationic lipid is about 1:125. In particular embodiments, the molar ratio of the chemically modified cationic lipid to the unmodified cationic lipid is about 1:130. In particular embodiments, the molar ratio of the chemically modified cationic lipid to the unmodified cationic lipid is about 1:135. In particular embodiments, the molar ratio of the chemically modified cationic lipid to the unmodified cationic lipid is about 1:140. In particular embodiments, the molar ratio of the chemically modified cationic lipid to the unmodified cationic lipid is about 1:145. In particular embodiments, the molar ratio of the chemically modified cationic lipid to the unmodified cationic lipid is about 1:149. In particular embodiments, the molar ratio of the chemically modified cationic lipid to the unmodified cationic lipid is about 1:150.

In some embodiments, the one or more phospholipids includes DSPC, or a derivative thereof. In some embodiments, the one or more phospholipids includes DOPE, or a derivative thereof. In some embodiments, the one or more phospholipids includes POPC, or a derivative thereof. In some embodiments, the one or more phospholipids includes DDPC, or a derivative thereof. In some embodiments, the one or more phospholipids includes DEPA-NA, or a derivative thereof. In some embodiments, the one or more phospholipids includes DEPC, or a derivative thereof. In some embodiments, the one or more phospholipids includes DEPE, or a derivative thereof. In some embodiments, the one or more phospholipids includes DEPG-NA, or a derivative thereof. In some embodiments, the one or more phospholipids includes DLOPC, or a derivative thereof. In some embodiments, the one or more phospholipids includes DLPA-NA, or a derivative thereof. In some embodiments, the one or more phospholipids includes DLPC, or a derivative thereof. In some embodiments, the one or more phospholipids includes DLPE, or a derivative thereof. In some embodiments, the one or more phospholipids includes DLPG-NA, or a derivative thereof. In some embodiments, the one or more phospholipids includes DLPG-NH4, or a derivative thereof. In some embodiments, the one or more phospholipids includes DLPS-NA, or a derivative thereof. In some embodiments, the one or more phospholipids includes DMPA-NA, or a derivative thereof. In some embodiments, the one or more phospholipids includes DMPC, or a derivative thereof. In some embodiments, the one or more phospholipids includes DMPE, or a derivative thereof. In some embodiments, the one or more phospholipids includes DMPG-NA, or a derivative thereof. In some embodiments, the one or more phospholipids includes DMPG-NH4, or a derivative thereof. In some embodiments, the one or more phospholipids includes DMPG-NH4/NA, or a derivative thereof. In some embodiments, the one or more phospholipids includes DOPA, or a derivative thereof. In some embodiments, the one or more phospholipids includes DOPG, or a derivative thereof. In some embodiments, the one or more phospholipids includes DOPS, or a derivative thereof. In some embodiments, the one or more phospholipids includes DPPA, or a derivative thereof. In some embodiments, the one or more phospholipids includes DPPC, or a derivative thereof. In some embodiments, the one or more phospholipids includes DPPE, or a derivative thereof. In some embodiments, the one or more phospholipids includes DPPG, or a derivative thereof. In some embodiments, the one or more phospholipids includes DPPS, or a derivative thereof. In some embodiments, the one or more phospholipids includes DSPA, or a derivative thereof. In some embodiments, the one or more phospholipids includes DSPG, or a derivative thereof. In some embodiments, the one or more phospholipids includes DSPS, or a derivative thereof. In some embodiments, the one or more phospholipids includes EPC, or a derivative thereof. In some embodiments, the one or more phospholipids includes HEPC, or a derivative thereof. In some embodiments, the one or more phospholipids includes MPPC, or a derivative thereof. In some embodiments, the one or more phospholipids includes MSPC, or a derivative thereof. In some embodiments, the one or more phospholipids includes PMPC, or a derivative thereof. In some embodiments, the one or more phospholipids includes POPC, or a derivative thereof. In some embodiments, the one or more phospholipids includes POPE, or a derivative thereof. In some embodiments, the one or more phospholipids includes PSPC, or a derivative thereof. In some embodiments, the one or more phospholipids includes SMPC, or a derivative thereof. In some embodiments, the one or more phospholipids includes SOPC, or a derivative thereof. In some embodiments, the one or more phospholipids includes SPPC, or a derivative thereof.

In some embodiments, the one or more steroids includes cholesterol, or a derivative thereof. In some embodiments, the one or more steroids include ergosterol, or a derivative thereof. In some embodiments, the one or more steroids include hopanoids, or a derivative thereof. In some embodiments, the one or more steroids include hydroxysteroid, or a derivative thereof. In some embodiments, the one or more steroids include phytosterol, or a derivative thereof. In some embodiments, the one or more steroids include zoosterol, or a derivative thereof. In some embodiments, the one or more steroids include gonane, or a derivative thereof. In some embodiments, the one or more steroids include testosterone, or a derivative thereof. In some embodiments, the one or more steroids include cholic acid, or a derivative thereof. In some embodiments, the one or more steroids include dexamethasone, or a derivative thereof. In some embodiments, the one or more steroids include lanosterol, or a derivative thereof. In some embodiments, the one or more steroids include progesterone, or a derivative thereof. In some embodiments, the one or more steroids include medrogestone, or a derivative thereof. In some embodiments, the one or more steroids include beta-sitosterol, or a derivative thereof. In some embodiments, the one or more steroids include cholestane, or a derivative thereof. In some embodiments, the one or more steroids include cholanes, or a derivative thereof. In some embodiments, the one or more steroids include pregnanes, or a derivative thereof. In some embodiments, the one or more steroids include androstanes, or a derivative thereof. In some embodiments, the one or more steroids include estranes, or a derivative thereof.

In certain embodiments, the one or more non-cationic lipids includes at least one chemically modified non-cationic lipid bound to the apolipoprotein, and at least one unmodified non-cationic lipid. In particular embodiments, the chemically modified non-cationic lipid is a steroid and the unmodified non-cationic lipid is also a steroid. In particular embodiments, the chemically modified non-cationic lipid is a phospholipid and the unmodified non-cationic lipid is also a phospholipid. In some embodiments, the molar ratio of the chemically modified non-cationic lipid to the unmodified non-cationic lipid is between about 1:1 and about 1:300. In some embodiments, the molar ratio of the chemically modified non-cationic lipid to the unmodified non-cationic lipid is between about 1:10 and about 1:200. In some embodiments, the molar ratio of the chemically modified non-cationic lipid to the unmodified non-cationic lipid is between about 1:25 and about 1:175. In some embodiments, the molar ratio of the chemically modified non-cationic lipid to the unmodified non-cationic lipid is between about 1:50 and about 1:150. In some embodiments, the molar ratio of the chemically modified non-cationic lipid to the unmodified non-cationic lipid is between about 1:100 and about 1:150. In some embodiments, the molar ratio of the chemically modified non-cationic lipid to the unmodified non-cationic lipid is between about 1:125 and about 1:150. In particular embodiments, the molar ratio of the chemically modified non-cationic lipid to the unmodified non-cationic lipid is about 1:50. In particular embodiments, the molar ratio of the chemically modified non-cationic lipid to the unmodified non-cationic lipid is about 1:60. In particular embodiments, the molar ratio of the chemically modified non-cationic lipid to the unmodified non-cationic lipid is about 1:70. In particular embodiments, the molar ratio of the chemically modified non-cationic lipid to the unmodified non-cationic lipid is about 1:80. In particular embodiments, the molar ratio of the chemically modified non-cationic lipid to the unmodified non-cationic lipid is about 1:90. In particular embodiments, the molar ratio of the chemically modified non-cationic lipid to the unmodified non-cationic lipid is about 1:100. In particular embodiments, the molar ratio of the chemically modified non-cationic lipid to the unmodified non-cationic lipid is about 1:105. In particular embodiments, the molar ratio of the chemically modified non-cationic lipid to the unmodified non-cationic lipid is about 1:110. In particular embodiments, the molar ratio of the chemically modified non-cationic lipid to the unmodified non-cationic lipid is about 1:115. In particular embodiments, the molar ratio of the chemically modified non-cationic lipid to the unmodified non-cationic lipid is about 1:120. In particular embodiments, the molar ratio of the chemically modified non-cationic lipid to the unmodified non-cationic lipid is about 1:125. In particular embodiments, the molar ratio of the chemically modified non-cationic lipid to the unmodified non-cationic lipid is about 1:130. In particular embodiments, the molar ratio of the chemically modified non-cationic lipid to the unmodified non-cationic lipid is about 1:135. In particular embodiments, the molar ratio of the chemically modified non-cationic lipid to the unmodified non-cationic lipid is about 1:140. In particular embodiments, the molar ratio of the chemically modified non-cationic lipid to the unmodified non-cationic lipid is about 1:145. In particular embodiments, the molar ratio of the chemically modified non-cationic lipid to the unmodified non-cationic lipid is about 1:149. In particular embodiments, the molar ratio of the chemically modified non-cationic lipid to the unmodified non-cationic lipid is about 1:150.

In some embodiments, the one or more lipid conjugates includes a pegylated lipid. In certain embodiments, the one or more lipid conjugates includes a DMG-PEG lipid, or a derivative thereof. In particular embodiments, the DMG-PEG lipid is DMG-PEG2000, or a derivative thereof. In particular embodiments, the DMG-PEG lipid is DMG-PEG5000, or a derivative thereof. In certain embodiments, the one or more lipid conjugates includes DMG-PEG1000, or a derivative thereof. In certain embodiments, the one or more lipid conjugates includes DSPE-PEG550, or a derivative thereof. In certain embodiments, the one or more lipid conjugates includes a DSPE-PEG lipid, or a derivative thereof. In particular embodiments, the DSPE-PEG lipid is DSPE-PEG5000. In certain embodiments, the one or more lipid conjugates includes DSPE-PEG2000, or a derivative thereof. In certain embodiments, the one or more lipid conjugates includes DSPE-PEG1000, or a derivative thereof. In certain embodiments, the one or more lipid conjugates includes DSPE-PEG550, or a derivative thereof. In certain embodiments, the one or more lipid conjugates includes a DMPE-PEG lipid, or a derivative thereof. In particular embodiments, the DMPE-PEG lipid is DMPE-PEG5000. In certain embodiments, the one or more lipid conjugates includes DMPE-PEG2000, or a derivative thereof. In certain embodiments, the one or more lipid conjugates includes DMPE-PEG1000, or a derivative thereof. In certain embodiments, the one or more lipid conjugates includes DMPE-PEG550, or a derivative thereof. In certain embodiments, the one or more lipid conjugates includes a C18-PEG lipid, or a derivative thereof. In particular embodiments, the C18-PEG lipid is C18-PEG5000. In certain embodiments, the one or more lipid conjugates includes C18-PEG2000, or a derivative thereof. In certain embodiments, the one or more lipid conjugates includes C18-PEG1000, or a derivative thereof. In certain embodiments, the one or more lipid conjugates includes C18-PEG550, or a derivative thereof. In certain embodiments, the one or more lipid conjugates includes a C16-PEG lipid, or a derivative thereof. In particular embodiments, the C16-PEG lipid is C16-PEG5000. In certain embodiments, the one or more lipid conjugates includes C16-PEG2000, or a derivative thereof. In certain embodiments, the one or more lipid conjugates includes C16-PEG1000, or a derivative thereof. In certain embodiments, the one or more lipid conjugates includes C16-PEG550, or a derivative thereof. In certain embodiments, the one or more lipid conjugates includes a C14-PEG lipid, or a derivative thereof. In particular embodiments, the C14-PEG lipid is C14-PEG5000. In certain embodiments, the one or more lipid conjugates includes C14-PEG2000, or a derivative thereof. In certain embodiments, the one or more lipid conjugates includes C14-PEG1000, or a derivative thereof. In certain embodiments, the one or more lipid conjugates includes C14-PEG550, or a derivative thereof. In certain embodiments, the one or more lipid conjugates includes a C12-PEG lipid, or a derivative thereof. In particular embodiments, the C12-PEG lipid is C12-PEG5000. In certain embodiments, the one or more lipid conjugates includes C12-PEG2000, or a derivative thereof. In certain embodiments, the one or more lipid conjugates includes C12-PEG1000, or a derivative thereof. In certain embodiments, the one or more lipid conjugates includes C12-PEG550, or a derivative thereof. In certain embodiments, the one or more lipid conjugates includes a cholesterol-PEG lipid, or a derivative thereof. In particular embodiments, the sterol-PEG lipid is cholesterol-PEG5000. In certain embodiments, the one or more lipid conjugates includes cholesterol-PEG2000, or a derivative thereof. In certain embodiments, the one or more lipid conjugates includes cholesterol-PEG1000, or a derivative thereof. In certain embodiments, the one or more lipid conjugates includes cholesterol-PEG550, or a derivative thereof.

In certain embodiments, the one or more lipid conjugates includes at least one chemically modified lipid conjugate bound to the apolipoprotein, and at least one unmodified lipid conjugate. In some embodiments, the molar ratio of the chemically modified lipid conjugate to the unmodified lipid conjugate is between about 1:1 and about 1:300. In some embodiments, the molar ratio of the chemically modified lipid conjugate to the unmodified lipid conjugate is between about 1:10 and about 1:200. In some embodiments, the molar ratio of the chemically modified lipid conjugate to the unmodified lipid conjugate is between about 1:25 and about 1:175. In some embodiments, the molar ratio of the chemically modified lipid conjugate to the unmodified lipid conjugate is between about 1:50 and about 1:150. In some embodiments, the molar ratio of the chemically modified lipid conjugate to the unmodified lipid conjugate is between about 1:100 and about 1:150. In some embodiments, the molar ratio of the chemically modified lipid conjugate to the unmodified lipid conjugate is between about 1:125 and about 1:150. In particular embodiments, the molar ratio of the chemically modified lipid conjugate to the unmodified lipid conjugate is about 1:50. In particular embodiments, the molar ratio of the chemically modified lipid conjugate to the unmodified lipid conjugate is about 1:60. In particular embodiments, the molar ratio of the chemically modified lipid conjugate to the unmodified lipid conjugate is about 1:70. In particular embodiments, the molar ratio of the chemically modified lipid conjugate to the unmodified lipid conjugate is about 1:80. In particular embodiments, the molar ratio of the chemically modified lipid conjugate to the unmodified lipid conjugate is about 1:90. In particular embodiments, the molar ratio of the chemically modified lipid conjugate to the unmodified lipid conjugate is about 1:100. In particular embodiments, the molar ratio of the chemically modified lipid conjugate to the unmodified lipid conjugate is about 1:105. In particular embodiments, the molar ratio of the chemically modified lipid conjugate to the unmodified lipid conjugate is about 1:110. In particular embodiments, the molar ratio of the chemically modified lipid conjugate to the unmodified lipid conjugate is about 1:115. In particular embodiments, the molar ratio of the chemically modified lipid conjugate to the unmodified lipid conjugate is about 1:120. In particular embodiments, the molar ratio of the chemically modified lipid conjugate to the unmodified lipid conjugate is about 1:125. In particular embodiments, the molar ratio of the chemically modified lipid conjugate to the unmodified lipid conjugate is about 1:130. In particular embodiments, the molar ratio of the chemically modified lipid conjugate to the unmodified lipid conjugate is about 1:135. In particular embodiments, the molar ratio of the chemically modified lipid conjugate to the unmodified lipid conjugate is about 1:140. In particular embodiments, the molar ratio of the chemically modified lipid conjugate to the unmodified lipid conjugate is about 1:145. In particular embodiments, the molar ratio of the chemically modified lipid conjugate to the unmodified lipid conjugate is about 1:149. In particular embodiments, the molar ratio of the chemically modified lipid conjugate to the unmodified lipid conjugate is about 1:150.

In certain embodiments, the one or more lipid conjugates includes a chemically modified DSPE-PEG lipid bound to the apolipoprotein, and an unmodified DMG-PEG lipid. In some embodiments, the one or more lipid conjugates includes a chemically modified DSPE-PEG2000 bound to the apolipoprotein, and an unmodified DMG-PEG2000.

In some embodiments, the one or more cationic lipids is DLin-MC3-DMA, the one or more phospholipids is DSPC, the one or more steroids is cholesterol, and the one or more lipid conjugates are pegylated lipids, wherein the pegylated lipids include unmodified DMG-PEG2000 and a chemically modified DSPE-PEG2000, comprising an amino group, that is bound to the apolipoprotein, wherein the apolipoprotein is ApoE.

In some embodiments, the one or more cationic lipids is DLin-MC3-DMA, the one or more phospholipids is DSPC, the one or more steroids is cholesterol, and the one or more lipid conjugates are pegylated lipids, wherein the pegylated lipids include unmodified DMG-PEG2000 and a chemically modified DSPE-PEG2000, comprising a carboxy group, that is bound to the apolipoprotein, wherein the apolipoprotein is ApoE.

In some embodiments, the one or more cationic lipids is DLin-MC3-DMA, the one or more phospholipids is DSPC, the one or more steroids is cholesterol, and the one or more lipid conjugates are pegylated lipids, wherein the pegylated lipids include unmodified DMG-PEG2000 and a chemically modified DSPE-PEG2000, comprising a maleimide group that is bound to the apolipoprotein, wherein the apolipoprotein is ApoE.

In some embodiments, the molar ratio of DLin-MC3-DMA, DPSC, cholesterol, and the one or more pegylated lipids is about 40:10:48.5:1.5, wherein the one or more pegylated lipids includes chemically modified DSPE-PEG2000 bound to ApoE and unmodified DMG-PEG2000, wherein the molar ratio of the chemically modified DSPE-PEG2000 to the unmodified DMG-PEG2000 is about 1:149.

In some embodiments, the molar ratio of DLin-MC3-DMA, DPSC, cholesterol, and the one or more pegylated lipids is about 50:10:38.5:1.5, wherein the one or more pegylated lipids includes chemically modified DSPE-PEG2000 bound to ApoE and unmodified DMG-PEG2000, wherein the molar ratio of the chemically modified DSPE-PEG2000 to the unmodified DMG-PEG2000 is about 1:149.

In some embodiments, the lipid nanoparticles have a size from about 50 nm to about 300 nm or from about 60 nm to about 120 nm.

In some embodiments, the polydispersity index of the lipid nanoparticles is less than about 0.3 or less than about 0.2.

In some embodiments, the zeta potential of the lipid nanoparticles is from about −40 mV to about 40 mV or from about −10 mV to about 10 mV.

In some embodiments, the molar ratio of the one or more cationic lipids to the one or more phospholipids is from about 1:1 to about 20:1. In some embodiments, the molar ratio of the one or more cationic lipids to the one or more phospholipids is from about 1.25:1 to about 14.9:1. In some embodiments, the molar ratio of the one or more cationic lipids to the one or more phospholipids is from about 4:1 to about 20:1. In some embodiments, the molar ratio of the one or more cationic lipids to the one or more phospholipids is from about 4:1 to about 10:1. In some embodiments, the molar ratio of the one or more cationic lipids to the one or more phospholipids is from about 10:1 to about 20:1. In some embodiments, the molar ratio of the one or more cationic lipids to the one or more phospholipids is from about 15:1 to about 20:1. In some embodiments, the molar ratio of the one or more cationic lipids to the one or more phospholipids is from about 2:1 to about 10:1. In some embodiments, the molar ratio of the one or more cationic lipids to the one or more phospholipids is from about 2.5:1 to about 9:1. In some embodiments, the molar ratio of the one or more cationic lipids to the one or more phospholipids is from about 2.5:1 to about 8:1. In some embodiments, the molar ratio of the one or more cationic lipids to the one or more phospholipids is about 4:1. In some embodiments, the molar ratio of the one or more cationic lipids to the one or more phospholipids is about 5:1.

In some embodiments, the molar ratio of the one or more cationic lipids to the one or more steroids is from about 0.25:1 to about 5:1. In some embodiments, the molar ratio of the one or more cationic lipids to the one or more steroids is from about 0.36:1 to about 3.73:1. In some embodiments, the molar ratio of the one or more cationic lipids to the one or more steroids is from about 0.5:1 to about 5:1. In some embodiments, the molar ratio of the one or more cationic lipids to the one or more steroids is from about 0.75:1 to about 5:1. In some embodiments, the molar ratio of the one or more cationic lipids to the one or more steroids is from about 2:1 to about 5:1. In some embodiments, the molar ratio of the one or more cationic lipids to the one or more steroids is from about 0.8:1 to about 2:1. In some embodiments, the molar ratio of the one or more cationic lipids to the one or more steroids is about 0.83:1. In some embodiments, the molar ratio of the one or more cationic lipids to the one or more steroids is about 1.3:1.

In some embodiments, the molar ratio of the one or more cationic lipids to the one or more lipid conjugates is from about 10:1 to about 1000:1. In some embodiments, the molar ratio of the one or more cationic lipids to the one or more lipid conjugates is from about 25:1 to about 1000:1. In some embodiments, the molar ratio of the one or more cationic lipids to the one or more lipid conjugates is from about 75:1 to about 1000:1. In some embodiments, the molar ratio of the one or more cationic lipids to the one or more lipid conjugates is from about 200:1 to about 1000:1. In some embodiments, the molar ratio of the one or more cationic lipids to the one or more lipid conjugates is from about 250:1 to about 1000:1. In some embodiments, the molar ratio of the one or more cationic lipids to the one or more lipid conjugates is from about 400:1 to about 1000:1. In some embodiments, the molar ratio of the one or more cationic lipids to the one or more lipid conjugates is from about 550:1 to about 1000:1. In some embodiments, the molar ratio of the one or more cationic lipids to the one or more lipid conjugates is from about 10:1 to about 745:1. In some embodiments, the molar ratio of the one or more cationic lipids to the one or more lipid conjugates is from about 20:1 to about 600:1. In some embodiments, the molar ratio of the one or more cationic lipids to the one or more lipid conjugates is from about 20:1 to about 800:1. In some embodiments, the molar ratio of the one or more cationic lipids to the one or more lipid conjugates is from about 25:1 to about 400:1. In some embodiments, the molar ratio of the one or more cationic lipids to the one or more lipid conjugates is from about 25:1 to about 800:1. In some embodiments, the molar ratio of the one or more cationic lipids to the one or more lipid conjugates is from about 25:1 to about 700:1. In some embodiments, the molar ratio of the one or more one or more cationic lipids to the one or more one or more lipid conjugates is about 26.67:1. In some embodiments, the molar ratio of the one or more one or more cationic lipids to the one or more one or more lipid conjugates is about 33.33:1.

In some embodiments, the molar ratio of the one or more steroids to the one or more lipid conjugates is from about 25:1 to about 750:1. In some embodiments, the molar ratio of the one or more steroids to the one or more lipid conjugates is from about 50:1 to about 750:1. In some embodiments, the molar ratio of the one or more steroids to the one or more lipid conjugates is from about 100:1 to about 750:1. In some embodiments, the molar ratio of the one or more steroids to the one or more lipid conjugates is from about 150:1 to about 750:1. In some embodiments, the molar ratio of the one or more steroids to the one or more lipid conjugates is from about 200:1 to about 750:1. In some embodiments, the molar ratio of the one or more steroids to the one or more lipid conjugates is from about 250:1 to about 750:1. In some embodiments, the molar ratio of the one or more steroids to the one or more lipid conjugates is from about 300:1 to about 750:1. In some embodiments, the molar ratio of the one or more steroids to the one or more lipid conjugates is from about 350:1 to about 750:1. In some embodiments, the molar ratio of the one or more steroids to the one or more lipid conjugates is from about 400:1 to about 750:1. In some embodiments, the molar ratio of the one or more steroids to the one or more lipid conjugates is from about 450:1 to about 750:1. In some embodiments, the molar ratio of the one or more steroids to the one or more lipid conjugates is from about 500:1 to about 750:1. In some embodiments, the molar ratio of the one or more steroids to the one or more lipid conjugates is from about 5:1 to about 750:1. In some embodiments, the molar ratio of the one or more steroids to the one or more lipid conjugates is from about 5:1 to about 700:1. In some embodiments, the molar ratio of the one or more steroids to the one or more lipid conjugates is from about 8:1 to about 750:1. In some embodiments, the molar ratio of the one or more steroids to the one or more lipid conjugates is from about 8:1 to about 700:1. In some embodiments, the molar ratio of the one or more steroids to the one or more lipid conjugates is from about 8:1 to about 699:1. In some embodiments, the molar ratio of the one or more steroids to the one or more lipid conjugates is from about 10:1 to about 500:1. In some embodiments, the molar ratio of the one or more steroids to the one or more lipid conjugates is from about 25:1 to about 500:1. In some embodiments, the molar ratio of the one or more steroids to the one or more lipid conjugates is about 25.67:1. In some embodiments, the molar ratio of the one or more steroids to the one or more lipid conjugates is about 32.33:1.

In some embodiments, the molar ratio of the one or more phospholipids to one or more lipid conjugates is from about 1:1 to about 300:1. In some embodiments, the molar ratio of the one or more phospholipids to one or more lipid conjugates is from about 1:1 to about 200:1. In some embodiments, the molar ratio of the one or more phospholipids to the one or more lipid conjugates is from about 50:1 to about 300:1. In some embodiments, the molar ratio of the one or more phospholipids to the one or more lipid conjugates is from about 100:1 to about 300:1. In some embodiments, the molar ratio of the one or more phospholipids to the one or more lipid conjugates is from about 125:1 to about 300:1. In some embodiments, the molar ratio of the one or more phospholipids to the one or more lipid conjugates is from about 150:1 to about 300:1. In some embodiments, the molar ratio of the one or more phospholipids to the one or more lipid conjugates is from about 175:1 to about 300:1. In some embodiments, the molar ratio of the one or more phospholipids to the one or more lipid conjugates is from about 200:1 to about 300:1. In some embodiments, the molar ratio of the one or more phospholipids to the one or more lipid conjugates is from about 225:1 to about 300:1. In some embodiments, the molar ratio of the one or more phospholipids to the one or more lipid conjugates is from about 250:1 to about 300:1. In some embodiments, the molar ratio of the one or more phospholipids to the one or more lipid conjugates is from about 275:1 to about 300:1. In some embodiments, the molar ratio of the one or more phospholipids to the one or more lipid conjugates is from about 3:1 to about 200:1. In some embodiments, the molar ratio of the one or more phospholipids to one or more lipid conjugates is from about 2:1 to about 200:1. In some embodiments, the molar ratio of the one or more phospholipids to the one or more lipid conjugates is from or about 5:1 to about 100:1. In some embodiments, the molar ratio of the one or more phospholipids to the one or more lipid conjugates is about 6.67:1.

In certain embodiments, the lipid nanoparticles do not comprise a biological targeting molecule having specificity for a cell surface antigen on a target cell. In some embodiments, the biological targeting molecule is an antibody, or antigen-binding fragment thereof. In certain embodiments, the cell surface antigen is present on a human immune cell. In some such embodiments, the human immune cell is a human T cell. In some such embodiments, the human immune cell is a human natural killer (NK) cell. In some such embodiments, the human immune cell is a human B cell. In some such embodiments, the human immune cell is a human macrophage. In other embodiments, the cell surface antigen is present on a human induced pluripotent stem cell (iPSC).

In some embodiments, the lipid nanoparticles comprise a nucleic acid.

In certain embodiments, the lipid nanoparticles comprise a cationic charge:phosphate ratio between the one or more cationic lipids and the nucleic acid of from about 1 to about 20. In certain embodiments, the lipid nanoparticles comprise a cationic charge:phosphate ratio between the one or more cationic lipids and the nucleic acid of from about 2 to about 16. In certain embodiments, the lipid nanoparticles comprise a cationic charge:phosphate ratio between the one or more cationic lipids and the nucleic acid of from about 4 to about 12. In certain embodiments, the lipid nanoparticles comprise a cationic charge:phosphate ratio between the one or more cationic lipids and the nucleic acid of from about 6 to about 10. In certain embodiments, the lipid nanoparticles comprise a cationic charge:phosphate ratio between the one or more cationic lipids and the nucleic acid of about 8.

In certain embodiments, the nucleic acid is an mRNA. In certain embodiments, the nucleic acid is a DNA molecule. In some embodiments, the DNA molecule is a double-stranded. DNA (dsDNA). In some embodiments, the DNA molecule is a recombinant DNA construct. In some embodiments, the DNA molecule is a single-stranded DNA (ssDNA). In certain embodiments, the nucleic acid is an RNA interference (RNAi) molecule. In certain embodiments, the nucleic acid is double-stranded (dsRNA). In certain embodiments, the nucleic acid is a mixture of RNAs. In certain embodiments, the nucleic acid is a mixture of RNA and DNA. In certain embodiments, the nucleic acid is an aptamer. In certain embodiments, the nucleic acid is an adjuvant. In certain embodiments, the nucleic acid is a decoy.

In some embodiments, the mRNA comprises a 5′ cap selected from the group consisting of an Anti-Reverse Cap Analog (ARCA) cap, a 7-methyl-guanosine (7 mG) cap, a CleanCap® analog, a vaccinia cap, and analogs thereof. In certain embodiments, the mRNA comprises at least one nucleoside modification. In some embodiments, the nucleoside modification is selected from the group consisting of a modification from uridine to pseudouridine and uridine to N1-methyl pseudouridine. In some embodiments, the nucleoside modification is from uridine to pseudouridine. In certain embodiments, the mRNA does not comprise a nucleoside modification.

In some embodiments, the mRNA encodes an engineered nuclease. In some embodiments, the DNA molecule (e.g., dsDNA) comprises a nucleic acid sequence encoding an engineered nuclease. In certain embodiments, the engineered nuclease is an engineered meganuclease. In certain embodiments, the engineered nuclease is a CRISPR system nuclease. In certain embodiments, the engineered nuclease is a TALEN. In certain embodiments, the engineered nuclease is a compact TALEN. In certain embodiments, the engineered nuclease is a zinc finger nuclease. In certain embodiments, the engineered nuclease is a megaTAL. In some embodiments, the engineered nuclease has specificity for a recognition sequence within a human T cell receptor (TCR) alpha gene. In some embodiments, the engineered nuclease has specificity for a recognition sequence within a human TCR alpha constant region (TRAC) gene. In some embodiments, the engineered nuclease has specificity for a recognition sequence within a human TCR beta gene. In some embodiments, the engineered nuclease has specificity for a recognition sequence within a human TCR beta constant region (TRBC) gene. In certain embodiments, the engineered nuclease is an engineered meganuclease that has specificity for a recognition sequence comprising SEQ ID NO: 2.

In some embodiments, the DNA molecule (e.g., dsDNA) comprises a donor template, wherein the donor template. In certain embodiments, the donor template comprises a nucleic acid sequence encoding a polypeptide of interest. In certain embodiments, the donor template comprises a 5′ homology arm and a 3′ homology arm. In some embodiments, the polypeptide of interest is a chimeric antigen receptor (CAR) or an exogenous TCR.

In certain embodiments, the RNAi molecule is a short hairpin RNA (shRNA). In certain embodiments, the RNAi molecule is a small interfering RNA (siRNA). In certain embodiments, the RNAi molecule is a hairpin siRNA. In certain embodiments, the RNAi molecule is a microRNA (miRNA). In certain embodiments, the RNAi molecule is a precursor miRNA. In certain embodiments, the RNAi molecule is an miRNA-adapted shRNA.

In some embodiments, the RNAi molecule is inhibitory against a component of the MHC class I molecule. In certain embodiments, the RNAi molecule is inhibitory against an MHC class I alpha-1 domain, alpha-2 domain, alpha-3 domain, or against beta-2 microglobulin. In particular embodiments, the RNAi molecule is inhibitory against beta-2 microglobulin. In particular embodiments, the RNAi molecule is inhibitory against CS1. In particular embodiments, the RNAi molecule is inhibitory against transforming growth factor-beta receptor 2 (TGFBR2). In particular embodiments, the RNAi molecule is inhibitory against Cbl proto-oncogene B (CBL-B). In particular embodiments, the RNAi molecule is inhibitory against CD52. In particular embodiments, the RNAi molecule is inhibitory against a TCR alpha gene. In particular embodiments, the RNAi molecule is inhibitory against a TRAC gene. In particular embodiments, the RNAi molecule is inhibitory against CD7. In particular embodiments, the RNAi molecule is inhibitory against glucocorticoid receptor (GR). In particular embodiments, the RNAi molecule is inhibitory against deoxycytidine kinase (DCK). In particular embodiments, the RNAi molecule is inhibitory against nuclear receptor subfamily 2 group F member 6 (NR2F6). In particular embodiments, the RNAi molecule is inhibitory against cytotoxic T-lymphocyte-associated protein 4 (CTLA-4). In particular embodiments, the RNAi molecule is inhibitory against C-C chemokine receptor type 5 (CCR5).

In another aspect, the invention provides a population of eukaryotic cells comprising a lipid nanoparticle composition described herein.

In some embodiments, the eukaryotic cells are human immune cells. In certain embodiments, the human immune cells are human T cells. In certain embodiments, the human immune cells are human NK cells. In certain embodiments, the human immune cells are human B cells. In certain embodiments, the human immune cells are human macrophages.

In some embodiments, the eukaryotic cells are human iPSCs.

In another aspect, the invention provides a method for transfecting a population of eukaryotic cells, the method comprising contacting the population of eukaryotic cells with a lipid nanoparticle composition described herein.

In some embodiments, the eukaryotic cells are human immune cells. In certain embodiments, the human immune cells are human T cells. In certain embodiments, the human immune cells are human NK cells. In certain embodiments, the human immune cells are human B cells. In certain embodiments, the human immune cells are human macrophages.

In some embodiments, the eukaryotic cells are human iPSCs.

In another aspect, the invention provides a method for introducing a nucleic acid into a population of eukaryotic cells, the method comprising contacting the population of eukaryotic cells with a lipid nanoparticle composition described herein wherein the lipid nanoparticles comprise the nucleic acid.

In certain embodiments, the lipid nanoparticles comprise a cationic charge:phosphate ratio between the one or more cationic lipids and the nucleic acid of from about 1 to about 20. In certain embodiments, the lipid nanoparticles comprise a cationic charge:phosphate ratio between the one or more cationic lipids and the nucleic acid of from about 2 to about 16. In certain embodiments, the lipid nanoparticles comprise a cationic charge:phosphate ratio between the one or more cationic lipids and the nucleic acid of from about 4 to about 12. In certain embodiments, the lipid nanoparticles comprise a cationic charge:phosphate ratio between the one or more cationic lipids and the nucleic acid of from about 6 to about 10. In certain embodiments, the lipid nanoparticles comprise a cationic charge:phosphate ratio between the one or more cationic lipids and the nucleic acid of about 8.

In some embodiments, the nucleic acid is an mRNA. In some embodiments, the nucleic acid is a DNA molecule. In some embodiments, the DNA molecule is a dsDNA. In some embodiments, the DNA molecule is a recombinant DNA construct. In some embodiments, the DNA molecule is an ssDNA. In some embodiments, the nucleic acid is an RNAi molecule. In some embodiments, the nucleic acid is a mixture of RNAs. In certain embodiments, the nucleic acid is a mixture of RNA and DNA. In certain embodiments, the nucleic acid is double-stranded (dsRNA). In certain embodiments, the nucleic acid is an aptamer. In certain embodiments, the nucleic acid is an adjuvant. In certain embodiments, the nucleic acid is a decoy.

In some embodiments, the mRNA comprises a 5′ cap selected from the group consisting of an Anti-Reverse Cap Analog (ARCA) cap, a 7-methyl-guanosine (7 mG) cap, a CleanCap® analog, a vaccinia cap, and analogs thereof. In some embodiments, the mRNA comprises at least one nucleoside modification. In some embodiments, the nucleoside modification is selected from the group consisting of a modification from uridine to pseudouridine and uridine to N1-methyl pseudouridine. In some embodiments, the nucleoside modification is from uridine to pseudouridine. In some embodiments, the mRNA does not comprise a nucleoside modification.

In certain embodiments, wherein the lipid nanoparticles comprise an mRNA, the mRNA encodes an engineered nuclease, wherein the engineered nuclease is expressed in the eukaryotic cells, and wherein the engineered nuclease binds and cleaves a recognition sequence in the genome of the eukaryotic cells to generate a cleavage site. In certain embodiments, wherein the lipid nanoparticles comprise a DNA molecule (e.g., a dsDNA), the DNA molecule comprises a nucleic acid sequence encoding an engineered nuclease, wherein the engineered nuclease is expressed in the eukaryotic cells, and wherein the engineered nuclease binds and cleaves a recognition sequence in the genome of the eukaryotic cells to generate a cleavage site. In some embodiments, the cleavage site is within a target gene.

In certain embodiments, the expression of a polypeptide encoded by the target gene is disrupted by non-homologous end joining at the cleavage site.

In some embodiments, the method further comprises introducing a second nucleic acid into the population of eukaryotic cells, wherein the second nucleic acid comprises a donor template. In some embodiments, the donor template is inserted into the cleavage site by homologous recombination. In some embodiments, the expression of a polypeptide encoded by the target gene is disrupted by insertion of the donor template into the cleavage site. In some embodiments, the donor template is flanked by a 5′ homology arm and a 3′ homology arm having homology to sequences 5′ upstream and 3′ downstream, respectively, of the cleavage site in the genome. In some embodiments, the donor template is introduced into the population of eukaryotic cells within 48 hours after the eukaryotic cells are contacted with the lipid nanoparticles described herein that comprise a nucleic acid encoding an engineered nuclease. In some embodiments, the donor template is introduced into the population of eukaryotic cells between 0-24 hours after the eukaryotic cells are contacted with the lipid nanoparticles described herein that comprise a nucleic acid encoding an engineered nuclease. In some embodiments, the donor template is introduced into the population of eukaryotic cells between 24-48 hours after the eukaryotic cells are contacted with the lipid nanoparticles described herein that comprise a nucleic acid encoding an engineered nuclease. In some embodiments, the donor template is introduced into the population of eukaryotic cells within 12 hours after the eukaryotic cells are contacted with the lipid nanoparticles described herein that comprise a nucleic acid encoding an engineered nuclease. In some embodiments, the donor template is introduced into the population of eukaryotic cells by a recombinant virus. In certain embodiments, the recombinant virus is a recombinant adeno-associated virus (AAV). In other embodiments, the donor template is introduced into the population of eukaryotic cells by a second lipid nanoparticle composition. In some such embodiments, the donor template is comprised by a recombinant DNA construct encapsulated by the second lipid nanoparticle composition. In particular embodiments, the second lipid nanoparticle composition comprises a lipid nanoparticle composition described herein.

In some embodiments, the donor template comprises a nucleic acid sequence encoding a polypeptide of interest. In certain embodiments, the polypeptide of interest is a CAR. In certain embodiments, the polypeptide of interest is an exogenous TCR.

In some embodiments, the target gene is a TCR alpha gene. In some embodiments, the target gene is a TRAC gene. In some embodiments, the target gene is a TCR beta gene. In some embodiments, the target gene is a TRBC gene.

In certain embodiments, the engineered nuclease is an engineered meganuclease. In certain embodiments, the engineered nuclease is a CRISPR system nuclease. In certain embodiments, the engineered nuclease is a TALEN. In certain embodiments, the engineered nuclease is a compact TALEN. In certain embodiments, the engineered nuclease is a zinc finger nuclease. In certain embodiments, the engineered nuclease is a megaTAL. In some embodiments, the engineered nuclease has specificity for a recognition sequence within a TCR alpha gene. In some embodiments, the engineered nuclease has specificity for a recognition sequence within a TRAC gene. In some embodiments, the engineered nuclease has specificity for a recognition sequence within a TCR beta gene. In some embodiments, the engineered nuclease has specificity for a recognition sequence within a TRBC gene. In certain embodiments, the engineered nuclease is an engineered meganuclease that has specificity for a recognition sequence comprising SEQ ID NO: 2. In particular embodiments, the donor template is inserted into the genome of the eukaryotic cells between positions 13 and 14 of SEQ ID NO: 2.

In some embodiments, wherein the nucleic acid is a DNA molecule (e.g., a dsDNA), the DNA molecule comprises a donor template. In certain embodiments, the donor template comprises a nucleic acid sequence encoding a polypeptide of interest. In particular embodiments, the DNA molecule is a recombinant DNA construct (i.e., a plasmid DNA).

In certain embodiments, the polypeptide of interest is a CAR. In certain embodiments, the polypeptide of interest is an exogenous TCR.

In some embodiments, the method further comprises introducing a second nucleic acid into the population of eukaryotic cells, wherein the second nucleic acid encodes an engineered nuclease, wherein the engineered nuclease is expressed in the eukaryotic cells, and wherein the engineered nuclease binds and cleaves a recognition sequence in the genome of the eukaryotic cells to generate a cleavage site. In some embodiments, the second nucleic acid encoding the engineered nuclease is an mRNA. In some embodiments, the mRNA is introduced using a lipid nanoparticle composition. In other embodiments, the mRNA is introduced by electroporation. In certain embodiments, the second nucleic acid encoding the engineered nuclease is introduced into the eukaryotic cells using a recombinant virus. In particular embodiments, the recombinant virus is a recombinant AAV. In some embodiments, the second nucleic acid is a recombinant DNA construct (e.g., a plasmid DNA) comprising a nucleic acid sequence encoding the engineered nuclease. In certain embodiments, the recombinant DNA construct is introduced by transfection. In certain embodiments, the recombinant DNA construct is introduced using a lipid nanoparticle composition, such as a lipid nanoparticle composition described herein.

In some embodiments, the cleavage site is within a target gene. In certain embodiments, the donor template is inserted into the cleavage site by homologous recombination. In some embodiments, the donor template is flanked by a 5′ homology arm and a 3′ homology arm having homology to sequences 5′ upstream and 3′ downstream, respectively, of the cleavage site in the genome. In some embodiments, the insertion of the donor template into the cleavage site disrupts expression of a polypeptide encoded by the target gene.

In some embodiments, the donor template is introduced into the population of eukaryotic cells within 48 hours after introduction of the nucleic acid encoding the engineered nuclease. In some embodiments, the donor template is introduced into the population of eukaryotic cells between 0-24 hours after introduction of the nucleic acid encoding the engineered nuclease. In some embodiments, the donor template is introduced into the population of eukaryotic cells between 24-48 hours after introduction of the nucleic acid encoding the engineered nuclease. In some embodiments, the donor template is introduced into the population of eukaryotic cells within 12 hours after introduction of the nucleic acid encoding the engineered nuclease.

In certain embodiments, the target gene is a TCR alpha gene. In some embodiments, the target gene is a TRAC gene. In certain embodiments, the target gene is a TCR beta gene. In some embodiments, the target gene is a TRBC gene.

In some embodiments, the engineered nuclease is an engineered meganuclease. In some embodiments, the engineered nuclease is a CRISPR system nuclease. In some embodiments, the engineered nuclease is a TALEN. In some embodiments, the engineered nuclease is a compact TALEN. In some embodiments, the engineered nuclease is a zinc finger nuclease. In some embodiments, the engineered nuclease is a megaTAL.

In certain embodiments, the engineered nuclease has specificity for a recognition sequence within a TCR alpha gene. In certain embodiments, the engineered nuclease has specificity for a recognition sequence within a TRAC gene. In certain embodiments, the engineered nuclease has specificity for a recognition sequence within a TCR beta gene. In certain embodiments, the engineered nuclease has specificity for a recognition sequence within a TRBC gene. In particular embodiments, the engineered nuclease is an engineered meganuclease that has specificity for a recognition sequence comprising SEQ ID NO: 2. In certain embodiments, the donor template is inserted into the genome of the eukaryotic cells between positions 13 and 14 of SEQ ID NO: 2.

In some embodiments, wherein the lipid nanoparticles comprise an RNAi molecule, the RNAi molecule is an shRNA. In some embodiments, the RNAi molecule is an siRNA. In some embodiments, the RNAi molecule is a hairpin siRNA. In some embodiments, the RNAi molecule is an miRNA. In some embodiments, the RNAi molecule is a precursor miRNA. In some embodiments, the RNAi molecule is an miRNA-adapted shRNA.

In some embodiments, the RNAi molecule is inhibitory against a component of the MHC class I molecule. In certain embodiments, the RNAi molecule is inhibitory against an MHC class I alpha-1 domain, alpha-2 domain, alpha-3 domain, or against beta-2 microglobulin. In particular embodiments, the RNAi molecule is inhibitory against beta-2 microglobulin. In particular embodiments, the RNAi molecule is inhibitory against CS1. In particular embodiments, the RNAi molecule is inhibitory against transforming growth factor-beta receptor 2 (TGFBR2). In particular embodiments, the RNAi molecule is inhibitory against Cbl proto-oncogene B (CBL-B). In particular embodiments, the RNAi molecule is inhibitory against CD52. In particular embodiments, the RNAi molecule is inhibitory against a TCR alpha gene. In particular embodiments, the RNAi molecule is inhibitory against a TRAC gene. In particular embodiments, the RNAi molecule is inhibitory against CD7. In particular embodiments, the RNAi molecule is inhibitory against glucocorticoid receptor (GR). In particular embodiments, the RNAi molecule is inhibitory against deoxycytidine kinase (DCK). In particular embodiments, the RNAi molecule is inhibitory against nuclear receptor subfamily 2 group F member 6 (NR2F6). In particular embodiments, the RNAi molecule is inhibitory against cytotoxic T-lymphocyte-associated protein 4 (CTLA-4). In particular embodiments, the RNAi molecule is inhibitory against C-C chemokine receptor type 5 (CCR5).

In certain embodiments, the eukaryotic cells are human immune cells. In some embodiments, the human immune cells are human T cells. In some embodiments, the human immune cells are human NK cells. In some embodiments, the human immune cells are human B cells. In some embodiments, the human immune cells are human macrophages. In some embodiments, the eukaryotic cells are human iPSCs.

In another aspect, the invention provides a population of eukaryotic cells prepared by any method described herein.

In another aspect, the invention provides a pharmaceutical composition comprising a pharmaceutically-acceptable carrier and the population of eukaryotic cells described herein.

In another aspect, the invention provides a method for reducing the number of target cells in a subject in need thereof, the method comprising: administering a therapeutically effective amount of a pharmaceutical composition described herein, wherein the population of eukaryotic cells are prepared as described herein to express a CAR or an exogenous TCR, and wherein the CAR or the exogenous TCR has specificity for an antigen present on the target cells.

In some embodiments, the eukaryotic cells are human T cells. In some embodiments, the eukaryotic cells are human NK cells. In some embodiments, the eukaryotic cells are human B cells. In some embodiments, the eukaryotic cells are human macrophages.

In certain embodiments, the eukaryotic cells express a CAR. In certain embodiments, the eukaryotic cells express an exogenous TCR.

In some embodiments, the method is a method of immunotherapy.

In certain embodiments, the target cells are cancer cells.

In certain embodiments, the method reduces the size of the cancer.

In some embodiments, the method eradicates the cancer in the subject.

In another aspect, the invention provides a kit for transfecting a population of eukaryotic cells comprising a lipid nanoparticle composition described herein.

In some embodiments, the kit further comprises a reagent that enhances the transfection efficiency of the lipid nanoparticle composition.

In another aspect, the invention provides a lipid nanoparticle composition described herein for use as a medicament.

In another aspect, the invention provides a lipid nanoparticle composition described herein for use in the manufacture of a medicament.

In some embodiments, the medicament is useful in the delivery of a nucleic acid to a target cell in a subject for the treatment of a disease.

In another aspect, the invention provides eukaryotic cells, or populations thereof, described herein for use as a medicament.

In another aspect, the invention provides eukaryotic cells, or populations thereof, described herein for use in the manufacture of a medicament.

In some embodiments, the medicament is useful for cancer immunotherapy in subjects in need thereof.

### BRIEF DESCRIPTION OF THE DRAWINGS

FIG. 1 provides a table of certain lipid nanoparticle compositions that are embodied by the invention. Each embodiment includes a total molar concentration of a cationic lipid (“Cat”), steroid (“Ster”), phospholipid (“PL”), and lipid conjugate (“LC”).

FIG. 2 provides flow cytometry data illustrating knockout of the endogenous TRAC gene by an engineered nuclease following delivery of the nuclease mRNA using LNPs having no conjugated ApoE, or LNPs that are modified to be conjugated to ApoE, in the presence or absence of human serum and soluble ApoE in the culture medium. Knockout of the TRAC gene is determined by CD3 expression on the cell surface. A) Unconjugated LNP in culture medium supplemented with soluble ApoE but no human serum. B) LNP conjugated to ApoE via a PEG lipid amino group, in culture medium supplemented with soluble ApoE but no human serum. C) LNP conjugated to ApoE via a PEG lipid carboxyl group, in culture medium supplemented with soluble ApoE but no human serum. D) LNP conjugated to ApoE via a PEG lipid maleimide group, in culture medium supplemented with soluble ApoE but no human serum. E) Unconjugated LNP in culture medium supplemented with human serum but not soluble ApoE. F) LNP conjugated to ApoE via a PEG lipid amino group, in culture medium supplemented with human serum but not soluble ApoE. G) LNP conjugated to ApoE via a PEG lipid carboxyl group, in culture medium supplemented with human serum but not soluble ApoE. H) LNP conjugated to ApoE via a PEG lipid maleimide group, in culture medium supplemented with human serum but not soluble ApoE.

FIG. 3 provides flow cytometry data illustrating knockout of the endogenous TRAC gene (X-axis) by an engineered nuclease following delivery of the nuclease mRNA using LNPs having no conjugated ApoE, or LNPs that are modified to be conjugated to ApoE, in the presence or absence of human serum and soluble ApoE in the culture medium. Knockout of the TRAC gene is determined by CD3 expression on the cell surface. The flow cytometry data further show knock-in of a transgene encoding a CAR (Y-axis), delivered by AAV, into the nuclease cleavage site. A) Unconjugated LNP in culture medium supplemented with soluble ApoE but no human serum. B) LNP conjugated to ApoE via a PEG lipid amino group, in culture medium supplemented with soluble ApoE but no human serum. C) LNP conjugated to ApoE via a PEG lipid carboxyl group, in culture medium supplemented with soluble ApoE but no human serum. D) LNP conjugated to ApoE via a PEG lipid maleimide group, in culture medium supplemented with soluble ApoE but no human serum. E) Unconjugated LNP in culture medium supplemented with human serum but not soluble ApoE. F) LINT conjugated to ApoE via a PEG lipid amino group, in culture medium supplemented with human serum but not soluble ApoE. G) LNP conjugated to ApoE via a PEG lipid carboxyl group, in culture medium supplemented with human serum but not soluble ApoE. H) LNP conjugated to ApoE via a PEG lipid maleimide group, in culture medium supplemented with human serum but not soluble ApoE.

FIG. 4 provides a table summarizing the flow cytometry results observed in FIG. 3.

### BRIEF DESCRIPTION OF THE SEQUENCES

SEQ ID NO: 1 sets forth the amino acid sequence of the wild-type I-CreI meganuclease from Chlamydomonas reinhardtii.

SEQ ID NO: 2 sets for the nucleic acid sequence of the TRC 1-2 recognition sequence (sense) for the TRC 1-2L.1592 meganuclease.

SEQ ID NO: 3 sets for the nucleic acid sequence of the TRC 1-2 recognition sequence (antisense) for the TRC 1-2L.1592 meganuclease.

SEQ ID NO: 4 sets for the amino acid sequence of the TRC 1-2L.1592 meganuclease.

### DETAILED DESCRIPTION OF THE INVENTION

### 1.1 References and Definitions

The patent and scientific literature referred to herein establishes knowledge that is available to those of skill in the art. The issued US patents, allowed applications, published foreign applications, and references, including GenBank database sequences, which are cited herein are hereby incorporated by reference to the same extent as if each was specifically and individually indicated to be incorporated by reference. All publications, patent applications, patents, and other references mentioned herein are incorporated by reference herein in their entirety.

The present invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment can be deleted from that embodiment. In addition, numerous variations and additions to the embodiments suggested herein will be apparent to those skilled in the art in light of the instant disclosure, which do not depart from the instant invention.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

As used herein, “a,” “an,” or “the” can mean one or more than one. For example, “a” cell can mean a single cell or a multiplicity of cells.

As used herein, unless specifically indicated otherwise, the word “or” is used in the inclusive sense of “and/or” and not the exclusive sense of “either/or.”

As used herein, the term “lipid” refers to a group of organic compounds that include, but are not limited to, esters of fatty acids and are characterized by being insoluble in water, but soluble in many organic solvents. Lipids are usually divided into at least three classes: (1) “simple lipids,” which include fats and oils as well as waxes; (2) “compound lipids,” which include phospholipids and glycolipids; and (3) “derived lipids” such as steroids. The selection of the individual lipid components of the lipid formulation is made to optimize delivery of a payload (e.g., a nucleic acid) to a target cell.

As used herein, the phrase “lipid formulation” refers to a formulation comprising one or more lipids (e.g., cationic lipids, non-cationic lipids, lipid conjugates, and the like).

As used herein, the phrase “lipid nanoparticle” refers to a microscopic lipid formulation that can be used to deliver an active agent or therapeutic agent, such as a nucleic acid (e.g., an mRNA, dsDNA), to a target site of interest (e.g., an immune cell). Lipid nanoparticles typically have a size of less than about 1000 nm.

As used herein, the phrase “lipid nanoparticle composition” refers to any composition comprising a lipid nanoparticle. Lipid nanoparticle compositions can comprise a lipid nanoparticle and an amount of apolipoprotein.

As used herein, the term “cationic lipid” refers to refers to any of a number of lipid species that carry a net positive charge at a selected pH, such as physiological pH (e.g., pH of about 7.0).

As used herein, the term “non-cationic lipid” refers to any neutral, zwitterionic, or anionic lipid.

As used herein, the term “steroid” refers to a class of hydrophobic, biologically active compounds comprising a specific 17-carbon fused ring system having three six membered rings and one five membered ring (a cyclopentanoperhydrophenanthrene ring system).

As used herein, a “phospholipid” refers a class of lipids whose molecule has a hydrophilic head containing a phosphate group, and two hydrophobic tails derived from fatty acids, joined by an alcohol residue.

As used herein, the term “lipid conjugate” refers to a conjugated lipid that inhibits aggregation of lipid particles.

As used herein, the term “anionic lipid” refers to any of a number of lipid species that carry a net negative charge at a selected pH, such as physiological pH.

The term “neutral lipid” refers to any of a number of lipid species that exist either in an uncharged or neutral zwitterionic form at a selected pH.

As used herein, the terms “chemically modified” or “chemical modification” or “modified”, in the context of a lipid component of a lipid nanoparticle, refers to an alteration of, or addition to, the chemical structure of a lipid to introduce a new functional group, thus generating a modified lipid. By way of example, the chemical structure of a lipid conjugate, such as a PEG lipid, can be chemically modified to comprise a functional moiety such as an amine group, which is capable of forming a covalent bond with an apolipoprotein (e.g., ApoE). Such chemical modifications can be made, for example, at the terminus of the lipid.

As used herein, the term “unmodified”, in the context of a lipid component of a lipid nanoparticle, refers to a parental form of a lipid prior to chemical modification (e.g., to introduce a functional group).

As used herein, the term “terminus” in the context of a lipid component of a lipid nanoparticle refers to the site on the modified lipid which is adjacent to the hydrophilic head group or the hydrophobic tail.

As used herein, the term “apolipoprotein” refers to a class of proteins that bind to and assist in solubilizing hydrophobic lipids and aiding in their transport. Apolipoproteins possess amphipathic (i.e., detergent-like) properties, and surround hydrophobic lipids to create a lipoprotein particle that is water soluble. Apolipoproteins are components of different lipoproteins and can be defined as non-exchangeable or exchangeable.

As used herein, the term “bound” refers to the presence of a chemical bond between two molecules (e.g., an apolipoprotein bound to a lipid). Such bonds can include, for example, covalent bonds, hydrophobic bonds, hydrophilic bonds, and noncovalent bonds.

As used herein, the term “zeta potential” refers to the overall charge that a nanoparticle acquires in a particular medium, and is a measure of electrostatic attraction and repulsion. Zeta potential values are indicative of dispersion stability, aggregation, and diffusion behavior. Zeta potential may be calculated from electrokinetic data obtained from, e.g., laser Doppler velocimetry. In this technique, a voltage is applied across a pair of electrodes at either end of a cell containing a nanoparticle dispersion. Charged nanoparticles are attracted to the oppositely charged electrode, and their velocity is measured and expressed in unit field strength as their electrophoretic mobility. Zeta values may be predictive in determining penetration through various cellular membranes.

As used herein, the term “polydispersity index” or “PDI” refers to the distribution of nanoparticle size and is a measure of uniformity. The polydispersity index is a unit-less measure which may be calculated from particle size data obtained according to techniques known in the art, for example, dynamic light scattering. Smaller values indicate a narrower size distribution, i.e., a more consistent particle size.

As used herein, the term “nuclease” or “endonuclease” refers to enzymes which cleave a phosphodiester bond within a polynucleotide chain.

As used herein, the terms “cleave” or “cleavage” refer to the hydrolysis of phosphodiester bonds within the backbone of a recognition sequence within a target sequence that results in a double-stranded break within the target sequence, referred to herein as a “cleavage site”.

As used herein, the term “meganuclease” refers to an endonuclease that binds double-stranded DNA at a recognition sequence that is greater than 12 base pairs. In some embodiments, the recognition sequence for a meganuclease of the present disclosure is 22 base pairs. A meganuclease can be, for example, an endonuclease that is derived from I-CreI (SEQ ID NO: 1), and can refer to an engineered variant of I-CreI that has been modified relative to natural I-CreI with respect to, for example, DNA-binding specificity, DNA cleavage activity, DNA-binding affinity, or dimerization properties. Methods for producing such modified variants of I-CreI are known in the art (e.g., WO 2007/047859, incorporated by reference in its entirety). A meganuclease as used herein binds to double-stranded DNA as a heterodimer. A meganuclease may also be a “single-chain meganuclease” in which a pair of DNA-binding domains is joined into a single polypeptide using a peptide linker. The term “homing endonuclease” is synonymous with the term “meganuclease.” Meganucleases of the present disclosure are substantially non-toxic when expressed in cells, such that cells can be transfected and maintained at 37° C. without observing deleterious effects on cell viability or significant reductions in meganuclease cleavage activity when measured using the methods described herein.

As used herein, the term “single-chain meganuclease” refers to a polypeptide comprising a pair of nuclease subunits joined by a linker. A single-chain meganuclease has the organization: N-terminal subunit-Linker-C-terminal subunit. The two meganuclease subunits will generally be non-identical in amino acid sequence and will recognize non-identical DNA sequences. Thus, single-chain meganucleases typically cleave pseudo-palindromic or non-palindromic recognition sequences. A single-chain meganuclease may be referred to as a “single-chain heterodimer” or “single-chain heterodimeric meganuclease” although it is not, in fact, dimeric. For clarity, unless otherwise specified, the teen “meganuclease” can refer to a dimeric or single-chain meganuclease.

As used herein, the term “linker” refers to an exogenous peptide sequence used to join two meganuclease subunits into a single polypeptide. A linker may have a sequence that is found in natural proteins, or may be an artificial sequence that is not found in any natural protein. A linker may be flexible and lacking in secondary structure or may have a propensity to form a specific three-dimensional structure under physiological conditions. A linker can include, without limitation, those encompassed by U.S. Pat. Nos. 8,445,251, 9,340,777, 9,434,931, and 10,041,053, each of which is incorporated by reference in its entirety. In some embodiments, a linker may have at least 80%, at least 85%, at least 90%, at least 91%, at least 92%, at least 93%, at least 94%, at least 95%, at least 96%, at least 97%, at least 98%, at least 99%, or more, sequence identity to residues 154-195 of SEQ ID NO: 4. In some embodiments, a linker may have an amino acid sequence comprising residues 154-195 of SEQ ID NO: 4.

As used herein, the term “TALEN” refers to an endonuclease comprising a DNA-binding domain comprising a plurality of TAL domain repeats fused to a nuclease domain or an active portion thereof from an endonuclease or exonuclease, including but not limited to a restriction endonuclease, horning endonuclease, S1 nuclease, mung bean nuclease, pancreatic DNAse I, micrococcal nuclease, and yeast HO endonuclease. See, for example, Christian et al. (2010) Genetics 186:757-761, which is incorporated by reference in its entirety. Nuclease domains useful for the design of TALENs include those from a Type IIs restriction endonuclease, including but not limited to FokI, FoM, StsI, HhaI, HindIII, Nod, BbvCI, EcoRI, BglI, and AlwI. Additional Type IIs restriction endonucleases are described in International Publication No. WO 2007/014275, which is incorporated by reference in its entirety. In some embodiments, the nuclease domain of the TALEN is a FokI nuclease domain or an active portion thereof. TAL domain repeats can be derived from the TALE (transcription activator-like effector) family of proteins used in the infection process by plant pathogens of the Xanthomonas genus. TAL domain repeats are 33-34 amino acid sequences with divergent 12th and 13th amino acids. These two positions, referred to as the repeat variable dipeptide (RVD), are highly variable and show a strong correlation with specific nucleotide recognition. Each base pair in the DNA target sequence is contacted by a single TAL repeat with the specificity resulting from the RVD. In some embodiments, the TALEN comprises 16-22 TAL domain repeats. DNA cleavage by a TALEN requires two DNA recognition regions (i.e., “half-sites”) flanking a nonspecific central region (i.e., the “spacer”). The teen “spacer” in reference to a TALEN refers to the nucleic acid sequence that separates the two nucleic acid sequences recognized and bound by each monomer constituting a TALEN. The TAL domain repeats can be native sequences from a naturally-occurring TALE protein or can be redesigned through rational or experimental means to produce a protein that binds to a pre-determined DNA sequence (see, for example, Boch et al. (2009) Science 326(5959):1509-1512 and Moscou and Bogdanove (2009) Science 326(5959):1501, each of which is incorporated by reference in its entirety). See also, U.S. Publication No. 20110145940 and International Publication No. WO 2010/079430 for methods for engineering a TALEN to recognize and bind a specific sequence and examples of RVDs and their corresponding target nucleotides. In some embodiments, each nuclease (e.g., FokI) monomer can be fused to a TAL effector sequence that recognizes and binds a different DNA sequence, and only when the two recognition sites are in close proximity do the inactive monomers come together to create a functional enzyme. It is understood that the term “TALEN” can refer to a single TALEN protein or, alternatively, a pair of TALEN proteins (i.e., a left TALEN protein and a right TALEN protein) which bind to the upstream and downstream half-sites adjacent to the TALEN spacer sequence and work in concert to generate a cleavage site within the spacer sequence. Given a predetermined DNA locus or spacer sequence, upstream and downstream half-sites can be identified using a number of programs known in the art (Kornel Labun; Tessa G. Montague; James A. Gagnon; Summer B. Thyme; Eivind Valen. (2016). CHOPCHOP v2: a web tool for the next generation of CRISPR genome engineering. Nucleic Acids Research; doi:10.1093/nar/gkw398; Tessa G. Montague; Jose M. Cruz; James A. Gagnon; George M. Church; Eivind Valen. (2014). CHOPCHOP: a CRISPR/Cas9 and TALEN web tool for genome editing. Nucleic Acids Res. 42. W401-W407). It is also understood that a TALEN recognition sequence can be defined as the DNA binding sequence (i.e., half-site) of a single TALEN protein or, alternatively, a DNA sequence comprising the upstream half-site, the spacer sequence, and the downstream half-site.

As used herein, the term “compact TALEN” refers to an endonuclease comprising a DNA-binding domain with one or more TAL domain repeats fused in any orientation to any portion of the I-TevI homing endonuclease or any of the endonucleases listed in Table 2 in U.S. Application No. 20130117869 (which is incorporated by reference in its entirety), including but not limited to MmeI, EndA, EndI, I-BasI, I-TevII, I-TevIII, I-TwoI, MspI, MvaI, NucA, and NucM. Compact TALENs do not require dimerization for DNA processing activity, alleviating the need for dual target sites with intervening DNA spacers. In some embodiments, the compact TALEN comprises 16-22 TAL domain repeats.

As used herein, the terms “zinc finger nuclease” or “ZFN” refers to a chimeric protein comprising a zinc finger DNA-binding domain fused to a nuclease domain from an endonuclease or exonuclease, including but not limited to a restriction endonuclease, homing endonuclease, S1 nuclease, mung bean nuclease, pancreatic DNAse I, micrococcal nuclease, and yeast HO endonuclease. Nuclease domains useful for the design of zinc finger nucleases include those from a Type IIs restriction endonuclease, including but not limited to FokI, FoM, and StsI restriction enzyme. Additional Type IIs restriction endonucleases are described in International Publication No. WO 2007/014275, which is incorporated by reference in its entirety. The structure of a zinc finger domain is stabilized through coordination of a zinc ion. DNA binding proteins comprising one or more zinc finger domains bind DNA in a sequence-specific manner. The zinc finger domain can be a native sequence or can be redesigned through rational or experimental means to produce a protein which binds to a pre-determined DNA sequence ˜18 basepairs in length, comprising a pair of nine basepair half-sites separated by 2-10 basepairs. See, for example, U.S. Pat. Nos. 5,789,538, 5,925,523, 6,007,988, 6,013,453, 6,200,759, and International Publication Nos. WO 95/19431, WO 96/06166, WO 98/53057, WO 98/54311, WO 00/27878, WO 01/60970, WO 01/88197, and WO 02/099084, each of which is incorporated by reference in its entirety. By fusing this engineered protein domain to a nuclease domain, such as FokI nuclease, it is possible to target DNA breaks with genome-level specificity. The selection of target sites, zinc finger proteins and methods for design and construction of zinc finger nucleases are known to those of skill in the art and are described in detail in U.S. Publications Nos. 20030232410, 20050208489, 2005064474, 20050026157, 20060188987 and International Publication No. WO 07/014275, each of which is incorporated by reference in its entirety. In the case of a zinc finger, the DNA binding domains typically recognize an 18-bp recognition sequence comprising a pair of nine basepair “half-sites” separated by a 2-10 basepair “spacer sequence”, and cleavage by the nuclease creates a blunt end or a 5′ overhang of variable length (frequently four basepairs). It is understood that the term “zinc finger nuclease” can refer to a single zinc finger protein or, alternatively, a pair of zinc finger proteins (i.e., a left ZFN protein and a right ZFN protein) that bind to the upstream and downstream half-sites adjacent to the zinc finger nuclease spacer sequence and work in concert to generate a cleavage site within the spacer sequence. Given a predetermined DNA locus or spacer sequence, upstream and downstream half-sites can be identified using a number of programs known in the art (Mandell J G, Barbas C F 3rd. Zinc Finger Tools: custom DNA-binding domains for transcription factors and nucleases. Nucleic Acids Res. 2006 Jul. 1; 34 (Web Server issue):W516-23). It is also understood that a zinc finger nuclease recognition sequence can be defined as the DNA binding sequence (i.e., half-site) of a single zinc finger nuclease protein or, alternatively, a DNA sequence comprising the upstream half-site, the spacer sequence, and the downstream half-site.

As used herein, the terms “CRISPR nuclease” or “CRISPR system nuclease” refers to a CRISPR (clustered regularly interspaced short palindromic repeats)-associated (Cas) endonuclease or a variant thereof, such as Cas9, that associates with a guide RNA that directs nucleic acid cleavage by the associated endonuclease by hybridizing to a recognition site in a polynucleotide. In certain embodiments, the CRISPR nuclease is a class 2 CRISPR enzyme. In some of these embodiments, the CRISPR nuclease is a class 2, type II enzyme, such as Cas9. In other embodiments, the CRISPR nuclease is a class 2, type V enzyme, such as Cpf1. The guide RNA comprises a direct repeat and a guide sequence (often referred to as a spacer in the context of an endogenous CRISPR system), which is complementary to the target recognition site. In certain embodiments, the CRISPR system further comprises a tracrRNA (trans-activating CRISPR RNA) that is complementary (fully or partially) to the direct repeat sequence (sometimes referred to as a tracr-mate sequence) present on the guide RNA. In particular embodiments, the CRISPR nuclease can be mutated with respect to a corresponding wild-type enzyme such that the enzyme lacks the ability to cleave one strand of a target polynucleotide, functioning as a nickase, cleaving only a single strand of the target DNA. Non-limiting examples of CRISPR enzymes that function as a nickase include Cas9 enzymes with a D10A mutation within the RuvC I catalytic domain, or with a H840A, N854A, or N863A mutation. Given a predetermined DNA locus, recognition sequences can be identified using a number of programs known in the art (Kornel Labun; Tessa G. Montague; James A. Gagnon; Summer B. Thyme; Eivind Valen. (2016). CHOPCHOP v2: a web tool for the next generation of CRISPR genome engineering. Nucleic Acids Research; doi:10.1093/nar/gkw398; Tessa G. Montague; Jose M. Cruz; James A. Gagnon; George M. Church; Eivind Valen. (2014). CHOPCHOP: a CRISPR/Cas9 and TALEN web tool for genome editing. Nucleic Acids Res. 42. W401-W407).

As used herein, the term “megaTAL” refers to a single-chain endonuclease comprising a transcription activator-like effector (TALE) DNA binding domain with an engineered, sequence-specific horning endonuclease.

As used herein, the terms “recombinant” or “engineered,” with respect to a protein, means having an altered amino acid sequence as a result of the application of genetic engineering techniques to nucleic acids that encode the protein and cells or organisms that express the protein. With respect to a nucleic acid, the term “recombinant” or “engineered” means having an altered nucleic acid sequence as a result of the application of genetic engineering techniques. Genetic engineering techniques include, but are not limited to, PCR and DNA cloning technologies; transfection, transformation, and other gene transfer technologies; homologous recombination; site-directed mutagenesis; and gene fusion. In accordance with this definition, a protein having an amino acid sequence identical to a naturally-occurring protein, but produced by cloning and expression in a heterologous host, is not considered recombinant or engineered.

As used herein, the term “wild-type” refers to the most common naturally occurring allele (i.e., polynucleotide sequence) in the allele population of the same type of gene, wherein a polypeptide encoded by the wild-type allele has its original functions. The term “wild-type” also refers to a polypeptide encoded by a wild-type allele. Wild-type alleles (i.e., polynucleotides) and polypeptides are distinguishable from mutant or variant alleles and polypeptides, which comprise one or more mutations and/or substitutions relative to the wild-type sequence(s). Whereas a wild-type allele or polypeptide can confer a normal phenotype in an organism, a mutant or variant allele or polypeptide can, in some instances, confer an altered phenotype. Wild-type nucleases are distinguishable from recombinant or non-naturally-occurring nucleases. The term “wild-type” can also refer to a cell, an organism, and/or a subject which possesses a wild-type allele of a particular gene, or a cell, an organism, and/or a subject used for comparative purposes.

As used herein, the term “genetically-modified” refers to a cell or organism in which, or in an ancestor of which, a genomic DNA sequence has been deliberately modified by recombinant technology. As used herein, the term “genetically-modified” encompasses the term “transgenic.”

As used herein with respect to recombinant proteins, the term “modification” means any insertion, deletion, or substitution of an amino acid residue in the recombinant sequence relative to a reference sequence (e.g., a wild-type or a native sequence).

As used herein, the terms “recognition sequence” or “recognition site” refers to a DNA sequence that is bound and cleaved by a nuclease. In the case of a meganuclease, a recognition sequence comprises a pair of inverted, 9 basepair “half sites” which are separated by four basepairs. In the case of a single-chain meganuclease, the N-terminal domain of the protein contacts a first half-site and the C-terminal domain of the protein contacts a second half-site. Cleavage by a meganuclease produces four basepair 3′ overhangs. “Overhangs,” or “sticky ends” are short, single-stranded DNA segments that can be produced by endonuclease cleavage of a double-stranded DNA sequence. In the case of meganucleases and single-chain meganucleases derived from I-CreI, the overhang comprises bases 10-13 of the 22 basepair recognition sequence. In the case of a compact TALEN, the recognition sequence comprises a first CNNNGN sequence that is recognized by the I-TevI domain, followed by a non-specific spacer 4-16 basepairs in length, followed by a second sequence 16-22 bp in length that is recognized by the TAL-effector domain (this sequence typically has a 5′ T base). Cleavage by a compact TALEN produces two basepair 3′ overhangs. In the case of a CRISPR nuclease, the recognition sequence is the sequence, typically 16-24 basepairs, to which the guide RNA binds to direct cleavage. Full complementarity between the guide sequence and the recognition sequence is not necessarily required to effect cleavage. Cleavage by a CRISPR nuclease can produce blunt ends (such as by a class 2, type II CRISPR nuclease) or overhanging ends (such as by a class 2, type V CRISPR nuclease), depending on the CRISPR nuclease. In those embodiments wherein a Cpf1 CRISPR nuclease is utilized, cleavage by the CRISPR complex comprising the same will result in 5′ overhangs and in certain embodiments, 5 nucleotide 5′ overhangs. Each CRISPR nuclease enzyme also requires the recognition of a PAM (protospacer adjacent motif) sequence that is near the recognition sequence complementary to the guide RNA. The precise sequence, length requirements for the PAM, and distance from the target sequence differ depending on the CRISPR nuclease enzyme, but PAMs are typically 2-5 base pair sequences adjacent to the target/recognition sequence. PAM sequences for particular CRISPR nuclease enzymes are known in the art (see, for example, U.S. Pat. No. 8,697,359 and U.S. Publication No. 20160208243, each of which is incorporated by reference in its entirety) and PAM sequences for novel or engineered CRISPR nuclease enzymes can be identified using methods known in the art, such as a PAM depletion assay (see, for example, Karvelis et al. (2017) Methods 121-122:3-8, which is incorporated herein in its entirety). In the case of a zinc finger, the DNA binding domains typically recognize an 18-bp recognition sequence comprising a pair of nine basepair “half-sites” separated by 2-10 basepairs and cleavage by the nuclease creates a blunt end or a 5′ overhang of variable length (frequently four basepairs).

As used herein, the term “target site” or “target sequence” refers to a region of the chromosomal DNA of a cell comprising a recognition sequence for a nuclease.

As used herein, the terms “DNA-binding affinity” or “binding affinity” means the tendency of a nuclease to non-covalently associate with a reference DNA molecule (e.g., a recognition sequence or an arbitrary sequence). Binding affinity is measured by a dissociation constant, Kd. As used herein, a nuclease has “altered” binding affinity if the Kd of the nuclease for a reference recognition sequence is increased or decreased by a statistically significant percent change relative to a reference nuclease.

As used herein, the term “specificity” means the ability of a nuclease to bind and cleave double-stranded DNA molecules only at a particular sequence of base pairs referred to as the recognition sequence, or only at a particular set of recognition sequences. The set of recognition sequences will share certain conserved positions or sequence motifs but may be degenerate at one or more positions. A highly-specific nuclease is capable of cleaving only one or a very few recognition sequences. Specificity can be determined by any method known in the art.

As used herein, the term “altered specificity,” when referencing to a nuclease, means that a nuclease binds to and cleaves a recognition sequence, which is not bound to and cleaved by a reference nuclease (e.g., a wild-type) under physiological conditions, or that the rate of cleavage of a recognition sequence is increased or decreased by a biologically significant amount (e.g., at least 2×, or 2×-10×) relative to a reference nuclease.

As used herein, the term “homologous recombination” or “HR” refers to the natural, cellular process in which a double-stranded DNA-break is repaired using a homologous DNA sequence as the repair template (see, e.g. Cahill et al. (2006), Front. Biosci. 11:1958-1976). The homologous DNA sequence may be an endogenous chromosomal sequence or an exogenous nucleic acid that was delivered to the cell.

As used herein, the term “non-homologous end-joining” or “NHEJ” refers to the natural, cellular process in which a double-stranded DNA-break is repaired by the direct joining of two non-homologous DNA segments (see, e.g. Cahill et al. (2006), Front. Biosci. 11:1958-1976). DNA repair by non-homologous end-joining is error-prone and frequently results in the untemplated addition or deletion of DNA sequences at the site of repair. In some instances, cleavage at a target recognition sequence results in NHEJ at a target recognition site. Nuclease-induced cleavage of a target site in the coding sequence of a gene followed by DNA repair by NHEJ can introduce mutations into the coding sequence, such as frameshift mutations, that disrupt gene function. Thus, engineered nucleases can be used to effectively knock-out a gene in a population of cells.

As used herein, the term “disrupted” or “disrupts” or “disrupts expression” or “disrupting a target sequence” refers to the introduction of a mutation (e.g., frameshift mutation) that interferes with the gene function and prevents expression and/or function of the polypeptide/expression product encoded thereby. For example, nuclease-mediated disruption of a gene can result in the expression of a truncated protein and/or expression of a protein that does not retain its wild-type function. Additionally, introduction of a donor template (i.e., a template nucleic acid) into a gene can result in no expression of an encoded protein, expression of a truncated protein, and/or expression of a protein that does not retain its wild-type function. Additionally, introduction of a donor template into a gene can result in no expression of an encoded protein, expression of a truncated protein, and/or expression of a protein that does not retain its wild-type function.

As used herein, “detectable cell-surface expression of an endogenous TCR” refers to the ability to detect one or more components of the TCR complex (e.g., an alpha/beta TCR complex) on the cell surface of an immune cell using standard experimental methods. Such methods can include, for example, immunostaining and/or flow cytometry specific for components of the TCR itself, such as a TCR alpha or TCR beta chain, or for components of the assembled cell-surface TCR complex, such as CD3. Methods for detecting cell-surface expression of an endogenous TCR (e.g., an alpha/beta TCR) on an immune cell include those described in the examples herein, and, for example, those described in MacLeod et al. (2017) Molecular Therapy 25(4): 949-961. Cells described herein having no detectable cell-surface expression of an endogenous protein are, therefore, cells in which an endogenous protein such as an endogenous TCR cannot be detected on the cell-surface by such methods.

As used herein, the term “chimeric antigen receptor” or “CAR” refers to an engineered receptor that confers or grafts specificity for an antigen onto an immune effector cell (e.g., a human T cell). A chimeric antigen receptor comprises at least an extracellular ligand-binding domain or moiety, a transmembrane domain, and an intracellular domain that comprises one or more signaling domains and/or co-stimulatory domains.

In some embodiments, the extracellular ligand-binding domain or moiety is an antibody, or antibody fragment. In this context, the term “antibody fragment” can refer to at least one portion of an antibody, that retains the ability to specifically interact with (e.g., by binding, steric hindrance, stabilizing/destabilizing, spatial distribution) an epitope of an antigen. Examples of antibody fragments include, but are not limited to, Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either YE or VH), camelid VHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (Fn3) (see U.S. Pat. No. 6,703,199, which describes fibronectin polypeptide minibodies).

In some embodiments, the extracellular ligand-binding domain or moiety is in the form of a single-chain variable fragment (scFv) derived from a monoclonal antibody, which provides specificity for a particular epitope or antigen (e.g., an epitope or antigen preferentially present on the surface of a cell, such as a cancer cell or other disease-causing cell or particle). In some embodiments, the scFv is attached via a linker sequence. In various embodiments, the extracellular ligand-binding domain is specific for any antigen or epitope of interest. In some embodiments, the scFv is murine, humanized, or fully human.

The extracellular ligand-binding domain of a chimeric antigen receptor can also comprise an autoantigen (see, Payne et al. (2016), Science 353 (6295): 179-184), that can be recognized by autoantigen-specific B cell receptors on B lymphocytes, thus directing T cells to specifically target and kill autoreactive B lymphocytes in antibody-mediated autoimmune diseases. Such CARs can be referred to as chimeric autoantibody receptors (CAARs), and their use is encompassed by the invention. The extracellular ligand-binding domain of a chimeric antigen receptor can also comprise a naturally-occurring ligand for an antigen of interest, or a fragment of a naturally-occurring ligand which retains the ability to bind the antigen of interest.

The intracellular stimulatory domain can include one or more cytoplasmic signaling domains that transmit an activation signal to the immune effector cell following antigen binding. Such cytoplasmic signaling domains can include, without limitation, a CD3 zeta signaling domain.

The intracellular stimulatory domain can also include one or more intracellular co-stimulatory domains that transmit a proliferative and/or cell-survival signal after ligand binding. Such intracellular co-stimulatory domains can be any of those known in the art and can include, without limitation, those co-stimulatory domains disclosed in WO 2018/067697 including, for example, Novel 6. Further examples of co-stimulatory domains can include 4-1BB (CD137), CD27, CD28, CD8, OX40, CD30, CD40, PD-1, ICOS, lymphocyte function-associated antigen-1 (LFA-1), CD2, CD7, LIGHT, NKG2C, B7-H3, a ligand that specifically binds with CD83, or any combination thereof.

A chimeric antigen receptor further includes additional structural elements, including a transmembrane domain that is attached to the extracellular ligand-binding domain via a hinge or spacer sequence. The transmembrane domain can be derived from any membrane-bound or transmembrane protein. For example, the transmembrane polypeptide can be a subunit of the T-cell receptor (e.g., an α, β, γ or ζ, polypeptide constituting CD3 complex), IL2 receptor p55 (a chain), p75 (β chain) or γ chain, subunit chain of Fc receptors (e.g., Fcy receptor III) or CD proteins such as the CD8 alpha chain. In certain examples, the transmembrane domain is a CD8 alpha domain. Alternatively, the transmembrane domain can be synthetic and can comprise predominantly hydrophobic residues such as leucine and valine.

The hinge region refers to any oligo- or polypeptide that functions to link the transmembrane domain to the extracellular ligand-binding domain. For example, a hinge region may comprise up to 300 amino acids, preferably 10 to 100 amino acids and most preferably 25 to 50 amino acids. Hinge regions may be derived from all or part of naturally occurring molecules, such as from all or part of the extracellular region of CD8, CD4 or CD28, or from all or part of an antibody constant region. Alternatively, the hinge region may be a synthetic sequence that corresponds to a naturally occurring hinge sequence or may be an entirely synthetic hinge sequence. In particular examples, a hinge domain can comprise a part of a human CD8 alpha chain, FcyRllla receptor or IgGl. In certain examples, the hinge region can be a CD8 alpha domain.

As used herein, the terms “exogenous T cell receptor” or “exogenous TCR” refer to a TCR whose sequence is introduced into the genome of an immune effector cell (e.g., a human T cell) that may or may not endogenously express the TCR. Expression of an exogenous TCR on an immune effector cell can confer specificity for a specific epitope or antigen (e.g., an epitope or antigen preferentially present on the surface of a cancer cell or other disease-causing cell or particle). Such exogenous T cell receptors can comprise alpha and beta chains or, alternatively, may comprise gamma and delta chains. Exogenous TCRs useful in the invention may have specificity to any antigen or epitope of interest.

As used herein, the terms “T cell receptor alpha gene” or “TCR alpha gene” refer to the locus in a T cell which encodes the T cell receptor alpha subunit. The T cell receptor alpha gene can refer to NCBI Gene ID No. 6955, before or after rearrangement. Following rearrangement, the T cell receptor alpha gene comprises an endogenous promoter, rearranged V and J segments, the endogenous splice donor site, an intron, the endogenous splice acceptor site, and the T cell receptor alpha constant region locus, which comprises the subunit coding exons.

As used herein, the term “T cell receptor alpha constant region gene” or “TCR alpha constant region gene” or “TRAC” refers to the coding sequence of the T cell receptor alpha gene. The TCR alpha constant region includes the wild-type sequence, and functional variants thereof, identified by NCBI Gene ID No. 28755.

As used herein, the term “T cell receptor beta gene” or “TCR beta gene” refer to the coding sequence of the T cell receptor beta gene. The TCR beta gene includes the wild-type sequence, and functional variants thereof, identified by NCBI Gene ID No. 6957.

As used herein, the term “recombinant DNA construct,” “recombinant construct,” “expression cassette,” “expression construct,” “chimeric construct,” “construct,” and “recombinant DNA fragment” are used interchangeably herein and are single or double-stranded polynucleotides. A recombinant construct comprises an artificial combination of nucleic acid fragments, including, without limitation, regulatory and coding sequences that are not found together in nature. For example, a recombinant DNA construct may comprise regulatory sequences and coding sequences that are derived from different sources, or regulatory sequences and coding sequences derived from the same source and arranged in a manner different than that found in nature. Such a construct may be used by itself or may be used in conjunction with a vector.

As used herein, the term “vector” or “recombinant DNA vector” may be a construct that includes a replication system and sequences that are capable of transcription and translation of a polypeptide-encoding sequence in a given host cell. If a vector is used, then the choice of vector is dependent upon the method that will be used to transform host cells as is well known to those skilled in the art. Vectors can include, without limitation, plasmid vectors and recombinant AAV vectors, or any other vector known in the art suitable for delivering a gene to a target cell. The skilled artisan is well aware of the genetic elements that must be present on the vector in order to successfully transform, select and propagate host cells comprising any of the isolated nucleotides or nucleic acid sequences of the invention.

In some embodiments, a “vector” also refers to a viral vector (i.e., a recombinant virus). Viral vectors can include, without limitation, retroviral vectors (i.e., recombinant retroviruses), lentiviral vectors (i.e., recombinant lentiviruses), adenoviral vectors (i.e., recombinant adenoviruses), and adeno-associated viral (AAV) vectors (i.e., recombinant AAVs).

As used herein, the term “immune cells” refers to cells isolated from a donor, particularly a human donor, which are known to mediate immune responses in the body. Immune cells can include, without limitation, T cells, such as CD4+ and CD8+ T cells, natural killer (NK) cells, B cells, gamma/delta. T cells, regulatory T cells, granulocytes, mast cells, monocytes, neutrophils, and dendritic cells.

As used herein, the term “human T cell” or “T cell” refers to a T cell isolated from a donor, particularly a human donor. T cells, and cells derived therefrom, include isolated T cells that have not been passaged in culture, T cells that have been passaged and maintained under cell culture conditions without immortalization, and T cells that have been immortalized and can be maintained under cell culture conditions indefinitely.

As used herein, the terms “human natural killer cell” or “human NK cell” or “natural killer cell” or “NK cell” refers to a type of cytotoxic lymphocyte critical to the innate immune system. The role NK cells play is analogous to that of cytotoxic T cells in the vertebrate adaptive immune response. NK cells provide rapid responses to virally infected cells and respond to tumor formation, acting at around 3 days after infection. Human NK cells, and cells derived therefrom, include isolated NK cells that have not been passaged in culture, NK cells that have been passaged and maintained under cell culture conditions without immortalization, and NK cells that have been immortalized and can be maintained under cell culture conditions indefinitely.

As used herein, the term “human B cell” or “B cell” refers to a B cell isolated from a donor, particularly a human donor. B cells, and cells derived therefrom, include isolated B cells that have not been passaged in culture, B cells that have been passaged and maintained under cell culture conditions without immortalization, and B cells that have been immortalized and can be maintained under cell culture conditions indefinitely.

As used herein, the term “human macrophage” refers to a macrophage cell isolated from a donor, particularly a human donor. Macrophages, and cells derived therefrom, include isolated macrophages that have not been passaged in culture, macrophages that have been passaged and maintained under cell culture conditions without immortalization, and macrophages that have been immortalized and can be maintained under cell culture conditions indefinitely.

As used herein, the term “induced pluripotent stem cell” or “iPSC” refers to types of pluripotent stem cells that can be generated directly from somatic cells.

As used herein, the term “biological targeting molecule” refers to biological molecules that selectively bind to molecules on the cell surface of target cells. Such targeting molecules can be attached to, anchored to, or otherwise incorporated into or on the surface of lipid nanoparticles in order to selectively bind the lipid nanoparticles to the target cells. Targeting molecules can include any peptides, nucleic acid molecules, or chemical compounds that selectively bind (i.e., have specificity for) molecules on the cell surface of target cells including, without limitation, antibodies, antibody fragments (e.g., single-chain variable fragments (scFvs), single-domain antibodies (sdAbs)), dual-affinity re-targeting antibodies (DARTs), aptamers, and the like. For example, a T cell targeting molecule has specificity for a molecule found on the cell surface of a T cell, thus enhancing the binding of a lipid nanoparticle comprising the T cell targeting molecule to a T cell. This term does not embrace apolipoproteins.

As used herein, a “control” or “control cell” refers to a cell that provides a reference point for measuring changes in genotype or phenotype of a genetically-modified cell. A control cell may comprise, for example: (a) a wild-type cell, i.e., of the same genotype as the starting material for the genetic alteration which resulted in the genetically-modified cell; (b) a cell of the same genotype as the genetically-modified cell but which has been transformed with a null construct (i.e., with a construct which has no known effect on the trait of interest); or, (c) a cell genetically identical to the genetically-modified cell but which is not exposed to conditions or stimuli or further genetic modifications that would induce expression of altered genotype or phenotype.

As used herein, the term “5′ cap” refers to a specially altered nucleotide on the 5′ end of primary transcripts such as messenger RNA. 5′ caps of mRNAs are important for RNA stability and processing, mRNA metabolism, the processing and maturation of an RNA transcript in the nucleus, transport of mRNA from the nucleus to the cytoplasm, mRNA stability, and efficient translation of mRNA to protein. A 5′ cap can be a naturally-occurring 5′ cap or one that differs from a naturally-occurring cap of an mRNA. 5′ caps useful for the disclosed method can include any 5′ caps known in the art.

As used herein, the term “nucleoside substitution” refers to the substitution of one or more naturally-occurring nucleosides of an mRNA to a modified nucleoside. Modified nucleosides useful for such substitutions are known in the art.

As used herein, the terms “treatment” or “treating a subject” refers to the administration of a genetically-modified immune cell or population of genetically-modified immune cells of the invention to a subject having a disease, disorder, or condition. For example, the subject can have a disease such as cancer, and treatment can represent immunotherapy for the treatment of the disease. Desirable effects of treatment include, but are not limited to, preventing occurrence or recurrence of disease, alleviation of symptoms, diminishment of any direct or indirect pathological consequences of the disease, decreasing the rate of disease progression, amelioration or palliation of the disease state, and remission or improved prognosis. In some aspects, a genetically-modified immune cell or population of genetically-modified immune cells described herein is administered during treatment in the form of a pharmaceutical composition of the invention.

The term “effective amount” or “therapeutically effective amount” refers to an amount sufficient to effect beneficial or desirable biological and/or clinical results. The therapeutically effective amount will vary depending on the formulation or composition used, the disease and its severity and the age, weight, physical condition and responsiveness of the subject to be treated. In specific embodiments, an effective amount of a genetically-modified immune cell or population of genetically-modified immune cells of the invention, or pharmaceutical compositions disclosed herein, reduces at least one symptom of a disease in a subject. In those embodiments wherein the disease is a cancer, an effective amount of the genetically-modified immune cells or pharmaceutical compositions disclosed herein reduces the level of proliferation or metastasis of cancer, causes a partial or full response or remission of cancer, or reduces at least one symptom of cancer in a subject.

As used herein, the term “cancer” should be understood to encompass any neoplastic disease (whether invasive or metastatic) which is characterized by abnormal and uncontrolled cell division causing malignant growth or tumor.

As used herein, the term “serum-free” refers to the use of liquid, solid, or liquid and solid culture media that lacks or is substantially free from serum (e.g., fetal bovine serum, calf bovine serum) for the growth of cells in culture.

As used herein, the term “exogenous” or “heterologous” in reference to a polynucleotide or nucleotide sequence is intended to mean a polynucleotide or sequence that is purely synthetic, that originates from a foreign species, or, if from the same species, is substantially modified from its native form in composition and/or genomic locus by deliberate human intervention.

As used herein, the recitation of a numerical range for a variable is intended to convey that the invention may be practiced with the variable equal to any of the values within that range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values of from about 0 to 2 can take the values 0, 1 or 2 if the variable is inherently discrete, and can take the values 0.0, 0.1, 0.01, 0.001, or any other real values and if the variable is inherently continuous.

### 2.1 Principle of the Invention

Without wishing to be bound by any particular theory, it has been discovered according to the present disclosure that use of certain lipid nanoparticles, which are bound to an apolipoprotein, can be used effectively for the delivery of nucleic acids into eukaryotic cells (e.g., T cells or NK cells), particularly in the presence of high serum concentrations, which can be optimal for the culture of such primary eukaryotic cells. Consequently, the lipid nanoparticle compositions described herein are useful, for example, in the genetic modification of such cells, or in the delivery of nucleic acids that can reduce the expression of endogenous proteins, while preventing several negative impacts typically associated with the delivery of nucleic acids by standard methods such as electroporation. It has been observed, for example, that lipid nanoparticles disclosed herein can be used for the delivery of mRNA encoding an engineered nuclease, resulting in up to a 5-fold increase in knock-out frequency of a target gene (e.g., the endogenous TRAC gene) in high serum conditions when compared to control lipid nanoparticles lacking an apolipoprotein, and nearly a 2-fold increase in knock-in frequency of a transgene (e.g., a CAR coding sequence) into the genome of human T cells.

Thus, provided herein are lipid nanoparticle compositions, and methods of using the same, which are bound to an apolipoprotein. The lipid nanoparticles described herein generally comprise one or more cationic lipids, one or more non-cationic lipids, one or more lipid conjugates, and an apolipoprotein bound to the one or more cationic lipids, the one or more non-cationic lipids, or the one or more lipid conjugates. Various bonds can exist between the lipids and the apolipoprotein, with particular examples having a covalent bond. Such covalent bonds can be enabled, for example, by a chemical modification of a lipid component to add a functional group capable of covalently binding to the apolipoprotein. In particular embodiments, the lipid nanoparticles described herein comprise a nucleic acid that can be delivered into a eukaryotic cell (e.g., a T cell or NK cell). Various types of nucleic acids can be delivered by the lipid nanoparticle compositions of the invention. Specific embodiments of the invention are described in detail herein below.

### 2.2 Lipid Nanoparticles

Described herein are lipid nanoparticles, and methods of using the same, that are bound to an apolipoprotein. A major characteristic of lipid nanoparticles is the fact that they are prepared with physiologically well-tolerated lipids. The lipid nanoparticles described herein generally comprise one or more cationic lipids, one or more non-cationic lipids, and one or more lipid conjugates, with an apolipoprotein bound to at least one lipid component.

### Cationic Lipids

Cationic lipids useful in the lipid nanoparticle compositions described herein can be, for example, DLin-DMA, (6Z,9Z,28Z,31Z)-heptatriacont-6,9,28,31-tetraene-19-yl 4-(dimethylamino)butanoate (DLin-MC3-DMA), 2-(2,2-di((9Z,12Z)-octadeca-9,12-dien-1-yl)-1,3-dioxolan-4-yl)-N,N-dimethylethan-1-amine (DLin-KC2-DMA), 1,2-dioleyloxy-3-dimethylaminopropan (DODMA), Bis[2-(4-{2-[4-(cis-9-octadecenoyloxy)phenylacetoxy]ethyl} piperidinyl)ethyl] disulfide (SS-OP), and derivatives thereof. DLin-MC3-DMA and derivatives thereof are described, for example, in WO 2010144740. DODMA and derivatives thereof are described, for example, in U.S. Pat. No. 7,745,651 and Mok et al. (1999), Biochimica et Biophysica. Acta, 1419(2): 137-150. DLin-DMA and derivatives thereof are described, for example, in U.S. Pat. No. 7,799,565. DLin-KC2-DMA and derivatives thereof are described, for example, in U.S. Pat. No. 9,139,554. SS-OP (NOF America Corporation, White Plains, NY) is described, for example, at www.nofamerica.com/store/index.php?dispatch=products.view&product_id=962. Additional and non-limiting examples of cationic lipids include methylpyridiyl-dialkyl acid (MPDACA), palmitoyl-oleoyl-nor-arginine (PONA), guanidino-dialkyl acid (GUADACA), 1,2-di-O-octadecenyl-3-trimethylammonium propane (DOTMA), 1,2-dioleoyl-3-trimethylammonium-propane (DOTAP), Bis {2-[N-methyl-N-(α-D-tocopherolhemisuccinatepropyl)amino]ethyl} disulfide (SS-33/3APO5), Bis {2-[4-(α-D-tocopherolhemisuccinateethyl)piperidyl]ethyl} disulfide (SS33/4PE15), Bis {2-[4-(cis-9-octadecenoateethyl)-1-piperidinyl]ethyl} disulfide (SS18/4PE16), Bis {2-[4-(cis,cis-9,12-octadecadienoateethyl)-1-piperidinyl]ethyl} disulfide (S S18/4PE13)1,2-dioleoyl-3-dimethylammonium-propane (DODAP), Di-octadecyl-amido-glycyl-spermine (DOGS), {2,3-dioleyloxy-N-[2(sperminecarboxamido)ethyl]-N,N-dimethyl-1-propanaminium trifluoroacetate} (DOSPA), 3β[N-(N′,N′-dimethylaminoethane)-carbamoyl]cholesterol (DC-Chol), N4-Cholesteryl-Spermine (GL-67), bis(guanidinium)-tris(2-aminoethyl)amine-cholesterol (BGTC), Dimethyldioctadecylammonium (DDAB), 1,2-dioleyloxypropyl)-3 dimethylhydroxyethyl ammoniumbromide (DORIE), 1,2-dimyristyloxypropyl-3-dimethyl-hydroxy ethyl ammonium bromide (DMRIE), N-(3-aminopro-pyl)-N,N-dimethyl-2,3-bis(dodecyloxy)-1-propanammonium bromide (GAP-DLRIE), N-t-butyl-N′-tetradecyl-3-tetradecylaminopropionamidine (diC14-amidine), di((Z)-non-2-en-1-yl) 9-((4-(dimethylamino)butanoyl)oxy)heptadecanedioate (L319), 1,1′-((2-(4-(2-((2-(bis(2-hydroxydodecyl)amino)ethyl)(2-hydroxydodecyl) amino)ethyl)piperazin-1-yl)ethyl)azanediyl)bis(dodecan-2-ol)(C12-200), OF-02, N1,N3,N5-tris(2-aminoethyl)benzene-1,3,5-tricarboxamide (TT3), ZA3-Ep10, or derivatives thereof.

The total molar concentration of the one or more cationic lipids in the lipid nanoparticles include those molar concentrations, and ranges of molar concentrations, described elsewhere herein for the one or more cationic lipids. In examples wherein the lipid nanoparticle comprises a nucleic acid, the molar ratio of the one or more cationic lipids to the nucleic acid can include those molar ratios described elsewhere herein for the one or more cationic lipids to the nucleic acid. %

### Non-Cationic Lipids

In particular embodiments, the total molar concentration of the non-cationic lipids includes those molar concentrations, and ranges of molar concentrations, described elsewhere herein for the one or more non-cationic lipids. Non-cationic lipids include, in some embodiments, one or more phospholipids and one or more steroids.

Phospholipids useful for the lipid nanoparticles described herein include, but are not limited to, 1,2-Distearoyl-sn-glycero-3-phosphocholine (DSPC), 1,2-Didecanoyl-sn-glycero-3-phosphocholine (DDPC), 1,2-Dierucoyl-sn-glycero-3-phosphate(Sodium Salt) (DEPA-NA), 1,2-Dierucoyl-sn-glycero-3-phosphocholine (DEPC), 1,2-Dierucoyl-sn-Oycero-3-phosphoethanolamine (DEPE), 1,2-Dierucoyl-sn-glycero-3[Phospho-rac-(1-glycerol)(Sodium Salt) (DEPG-NA), 1,2-Dilinoleoyl-sn-glycero-3-phosphocholine (DLOPC), 1,2-Dilauroyl-sn-glycero-3-phosphate(Sodium Salt) (DLPA-NA), 1,2-Dilauroyl-sn-glycero-3-phosphocholine (DLPC), 1,2-Dilauroyl-sn-Oycero-3-phosphoethanolamine (DLPE), 1,2-Dilauroyl-sn-glycero-3[Phospho-rac-(1-glycerol . . . )(Sodium Salt) (DLPG-NA), 1,2-Dilauroyl-sn-glycero-3[Phospho-rac-(1-glycerol)(Ammonium Salt) (DLPG-NH4), 1,2-Dilauroyl-sn-glycero-3-phosphoserine(Sodium Salt) (DLPS-NA), 1,2-Dimyristoyl-sn-glycero-3-phosphate(Sodium Salt) (DMPA-NA), 1,2-Dimyristoyl-sn-glycero-3-phosphocholine (DMPC), 1,2-Dimyristoyl-sn-glycero-3-phosphoethanolamine (DMPE), 1,2-Dimyristoyl-sn-glycero-3[Phospho-rac-(1-glycerol)(Sodium Salt) (DMPG-NA), 1,2-Dimyristoyl-sn-glycero-3[Phospho-rac-(1-glycerol)(Ammonium Salt) (DMPG-NH4), 1,2-Dimyristoyl-sn-2-lycero-3[Phospho-rac-(1-glycerol)(Sodium/Ammonium Salt) (DMPG-NH4/NA), 1,2-Dimyristoyl-sn-glycero-3-phosphoserine(Sodium Salt) (DMPS-NA), 1,2-Dioleoyl-sn-glycero-3-phosphate(Sodium Salt) (DOPA-NA), 1,2-Dioleoyl-sn-glycero-3-phosphocholine (DOPC), 1,2-Dioleoyl-sn-glycero-3-phosphoethanolamine (DOPE), 1,2-Dioleoyl-sn-glycero-3[Phospho-rac-(1-glycerol)(Sodium Salt) (DOPG-NA), 1,2-Dioleoyl-sn-glycero-3-phosphoserine(Sodium Salt) (DOPS-NA), 1,2-Dipalmitoyl-sn-glycero-3-phosphate(Sodium Salt) (DPPA-NA), 1,2-Dipalmitoyl-sn-glycero-3-phosphocholine (DPPC), 1,2-Dipalmitoyl-sn-glycero-3-phosphoethanolamine (DPPE), 1,2-Dipalmitoyl-sn-glycero-3[Phospho-rac-(1-glycerol)(Sodium Salt) (DPPG-NA), 1,2-Dipalmitoyl-sn-glycero-3[Phospho-rac-(1-glycerol)(Ammonium Salt) (DPPG-NH4), 1,2-Dipalmitoyl-sn-glycero-3-phosphoserine(Sodium Salt) (DPPS-NA), 1,2-Distearoyl-sn-glycero-3-phosphate(Sodium Salt) (DSPA-NA), 1,2-Distearoyl-sn-glycero-3-phosphoethanolamine (DSPE), 1,2-Distearoyl-sn-glycero-3[Phospho-rac-(1-glycerol)(Sodium Salt) (DSPG-NA), 1,2-Distearoyl-sn-glycero-3[Phospho-rac-(1-glycerol)(Ammonium Salt) (DSPG-NH4), 1,2-Distearoyl-sn-glycero-3-phosphoserine(Sodium Salt) (DSPS-NA), Egg-PC (EPC), Hydrogenated Egg PC (HEPC). Hydrogenated Soy PC (HSPC), 1-Myristoyl-sn-glycero-3-phosphocholine (LYSOPCMYRISTIC), 1-Paimitoyl-sn-glycero-3-phosphocholine (LYSOPCPALMITIC), 1-Stearoyl-sn-glycero-3-phosphocholine (LYSOPCSTEARIC), 1-Myristoyl-2-palmitoyl-sn-glycero3-phosphocholine (MPPC), 1-Myristoyl-2-stearoyl-sn-glycero-3-phosphocholine (MSPC), 1-Palmitoyl-2-myristoyl-sn-Oycero-3-phosphocholine (PMPC), 1-Palmitoyl-2-oleoyl-sn-glycero-3-phosphocholine (POPC), 1-Palmitoyl-2-oleoyl-sn-glycero-3-phosphoethanolamine (POPE), 1-Palmitoyl-2-oleoyl-sn-glycero-3[Phospho-rac-(1-glycerol)](Sodium Salt) (POPG-NA), 1-Palmitoyl-2-stearoyl-sn-glycero-3-phosphocholine (PSPC), 1-Stearoyl-2-myristoyl-sn-glycero-3-phosphocholine (SMPC), 1-Stearoyl-2-oleoyl-sn-glycero-3-phosphocholine (SOPC), and 1-Stearoyl-2-palmitoyl-sn-glycero-3-phosphocholine (SPPC). In particular embodiments, the phospholipid is DSPC. In particular embodiments, the phospholipid is DOPE. In particular embodiments, the phospholipid is DOPC.

In some embodiments, the total molar concentration of the one or more phospholipids includes those molar concentrations, and ranges of molar concentrations, described elsewhere herein for the one or more phospholipids.

In some embodiments, the non-cationic lipids comprised by the lipid nanoparticles include one or more steroids. Steroids useful for the lipid nanoparticles described herein include, but are not limited to, cholestanes such as cholesterol, cholanes such as cholic acid, pregnanes such as progesterone, androstanes such as testosterone, and estranes such as estradiol. Further steroids include, but are not limited to, cholesterol (ovine), cholesterol sulfate, desmosterol-d6, cholesterol-d7, lathosterol-d7, desmosterol, stigmasterol, lanosterol, dehydrocholesterol, dihydrolanosterol, zymosterol, lathosterol, zymosterol-d5, 14-demethyl-lanosterol, 14-demethyl-lanosterol-d6, 8(9)-dehydrocholesterol, 8(14)-dehydrocholesterol, diosgenin, DHEA sulfate, DHEA, lanosterol-d6, dihydrolanosterol-d7, campesterol-d6, sitosterol, lanosterol-95, Dihydro FF-MAS-d6, zymostenol-d7, zymostenol, sitostanol, campestanol, campesterol, 7-dehydrodesmosterol, pregnenolone, sitosterol-d7, Dihydro T-MAS, Delta 5-avenasterol, Brassicasterol, Dihydro FF-MAS, 24-methylene cholesterol, cholic acid derivatives, cholesteryl esters, and glycosylated sterols. Additional steroids include, without limitation, ergosterol, hopanoids, hydroxysteroid, phytosterol, zoosterol, gonane, dexamethasone, medrogestone, or beta-sitosterol. In particular embodiments, the lipid nanoparticles comprise cholesterol.

In some embodiments, the total molar concentration of the one or more steroids includes those molar concentrations, and ranges of molar concentrations, described elsewhere herein for the one or more steroids.

### Lipid Conjugates

Lipid conjugates useful for the lipid nanoparticles described herein include, but are not limited to, ceramide PEG derivatives such as C8 PEG2000 ceramide, C16 PEG2000 ceramide, C8 PEG5000 ceramide, C16 PEG5000 ceramide, C8 PEG750 ceramide, and C16 PEG750 ceramide. The lipid conjugates can include phosphoethanolamine PEG derivatives, such as 16:0 PEG5000 PE, 14:0 PEG5000 PE, 18:0 PEG5000 PE, 18:1 PEG5000 PE, 16:0 PEG3000 PE, 14:0 PEG3000 PE, 18:0 PEG3000 PE, 18:1 PEG3000 PE, 16:0 PEG2000 PE, 14:0 PEG2000 PE, 18:0 PEG2000 PE, 18:1 PEG2000 PE 16:0 PEG1000 PE, 14:0 PEG1000 PE, 18:0 PEG1000 PE, 18:1 PEG1000 PE, 16:0 PEG750 PE, 14:0 PEG750 PE, 18:0 PEG750 PE, 18:1 PEG750 PE, 16:0 PEG550 PE, 14:0 PEG550 PE, 18:0 PEG550 PE, 18:1 PEG550 PE, 16:0 PEG350 PE, 14:0 PEG350 PE, 18:0 PEG350 PE, and 18:1 PEG350. The lipid conjugates can include sterol PEG derivatives such as Chol-PEG600. The lipid conjugates can also include glycerol PEG derivatives such as DMG-PEG5000, DSG-PEG5000, DPG-PEG5000, DMG-PEG3000, DSG-PEG3000, DPG-PEG3000, DMG-PEG2000, DSG-PEG2000, DPG-PEG2000, DMG-PEG1000, DSG-PEG1000, DPG-PEG1000, DMG-PEG750, DSG-PEG750, DPG-PEG750, DMG-PEG550, DSG-PEG550, DPG-PEG550, DMG-PEG350, DSG-PEG350, and DPG-PEG350. The lipid conjugates can further include phospholipid PEG derivatives such as DSPE-PEG5000, DSPE-PEG2000, DSPE-PEG1000, or DSPE-PEG550.

In some embodiments, the total molar concentration of the one or more lipid conjugates includes those molar concentrations, and ranges of molar concentrations, described elsewhere herein for the one or more lipid conjugates.

### Lipid Nanoparticle Compositions

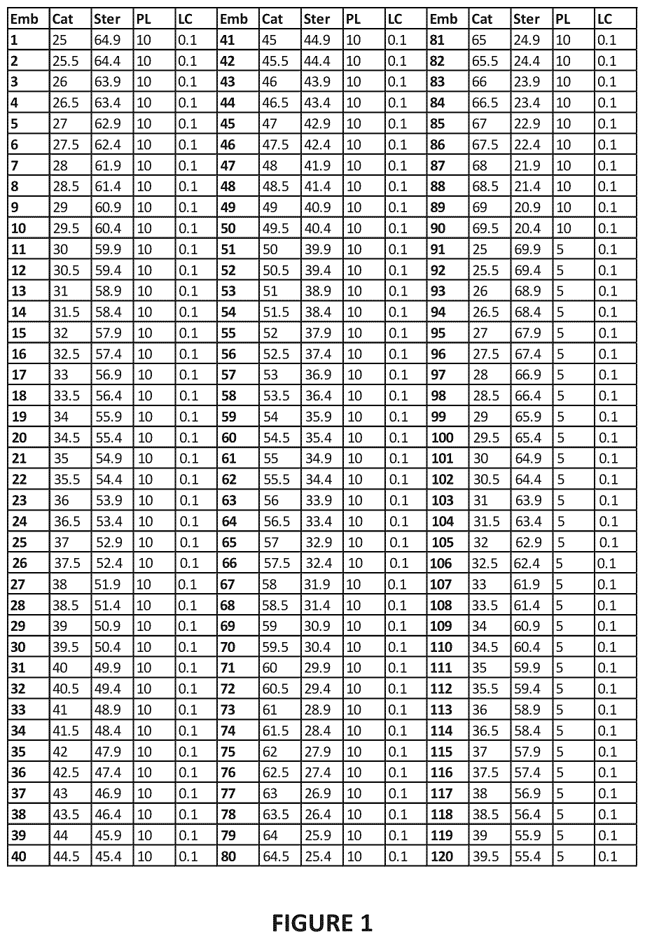

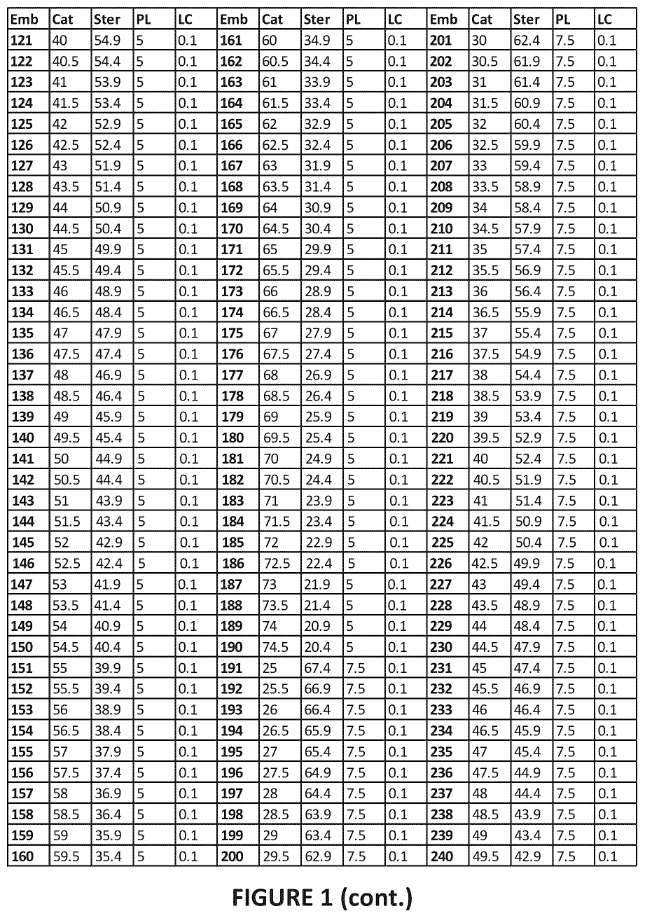

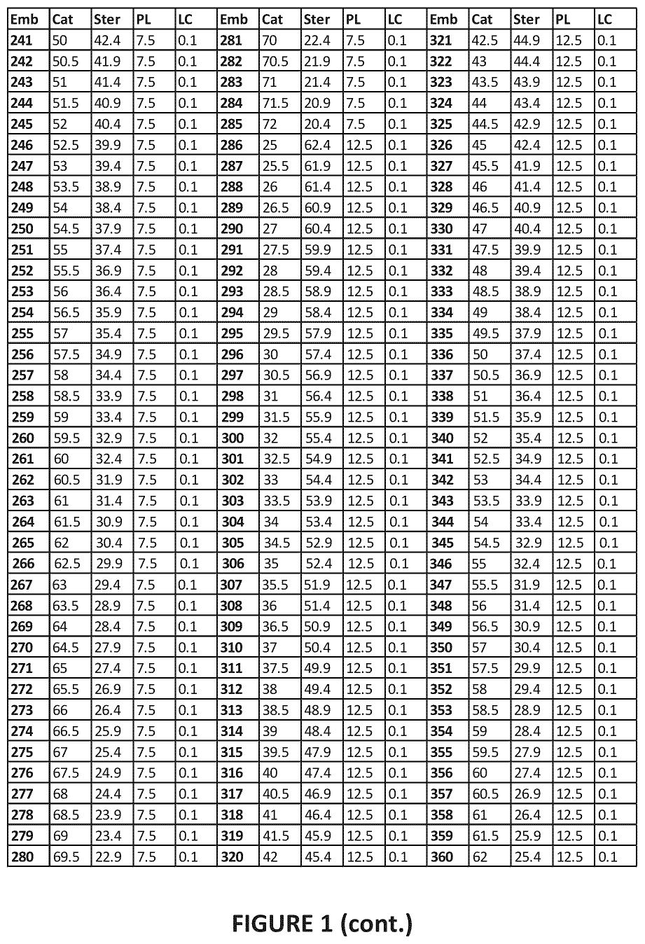

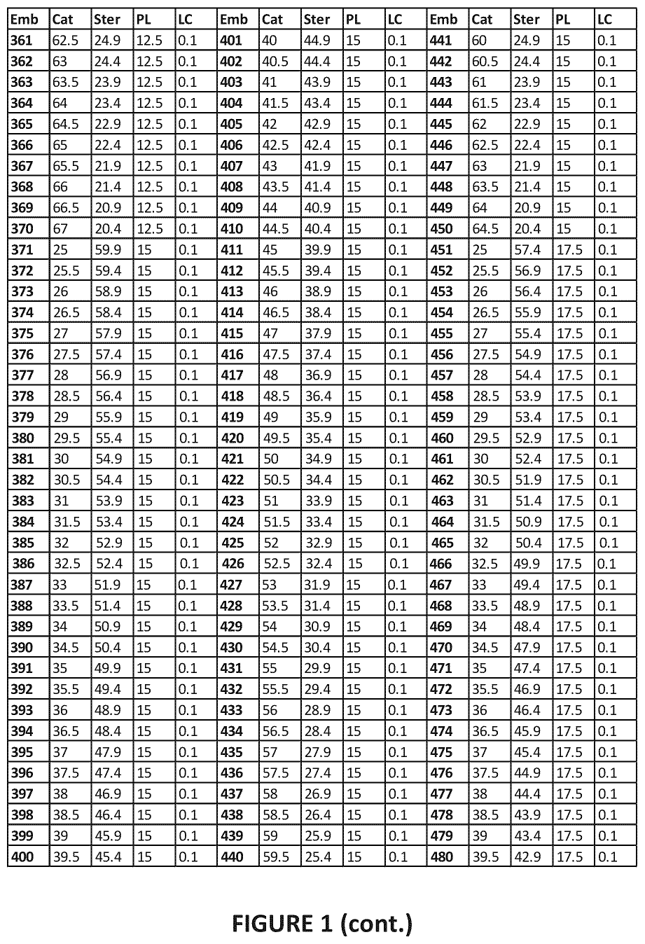

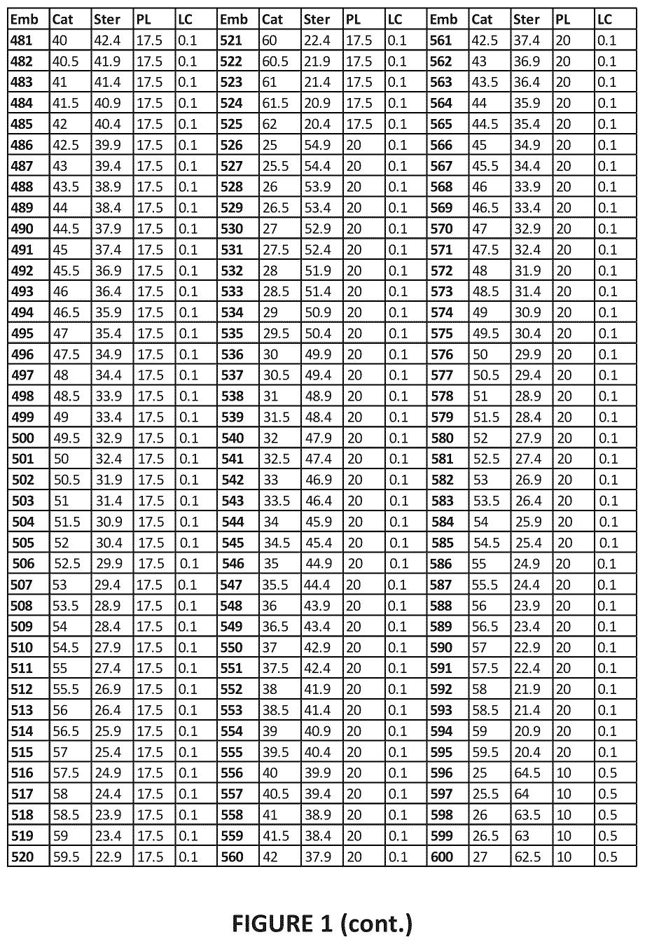

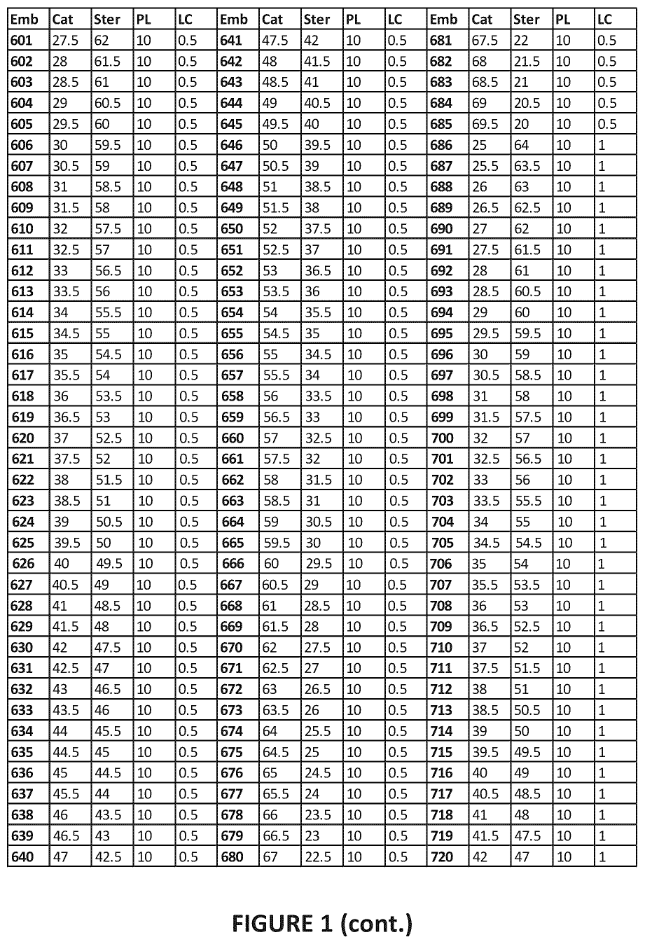

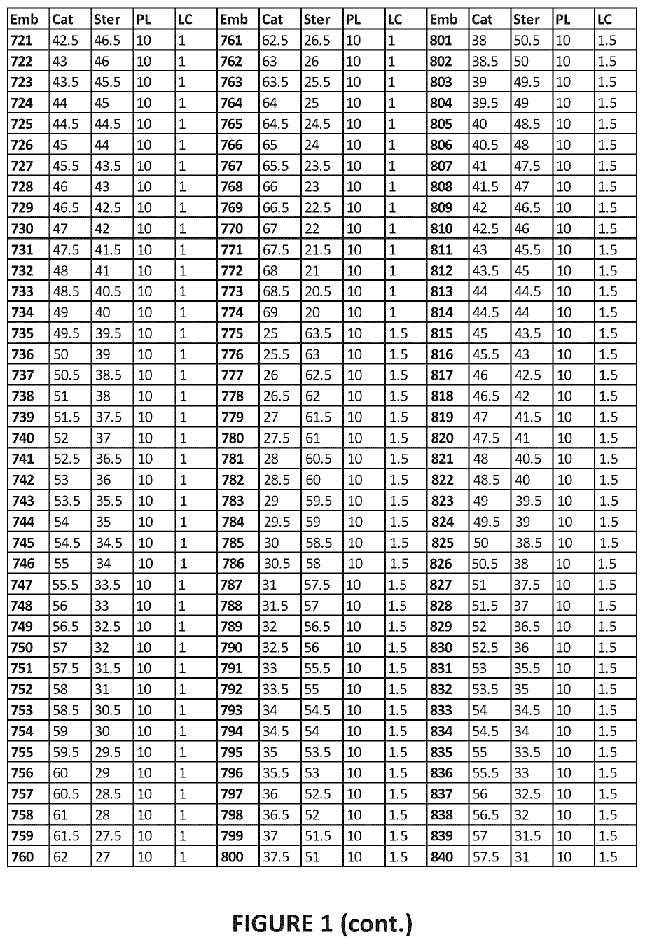

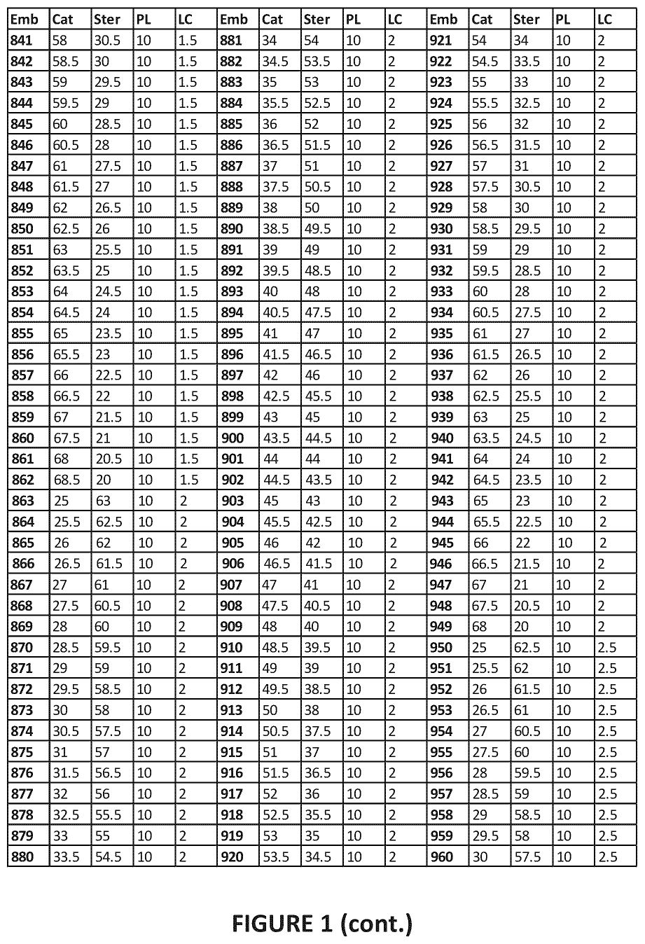

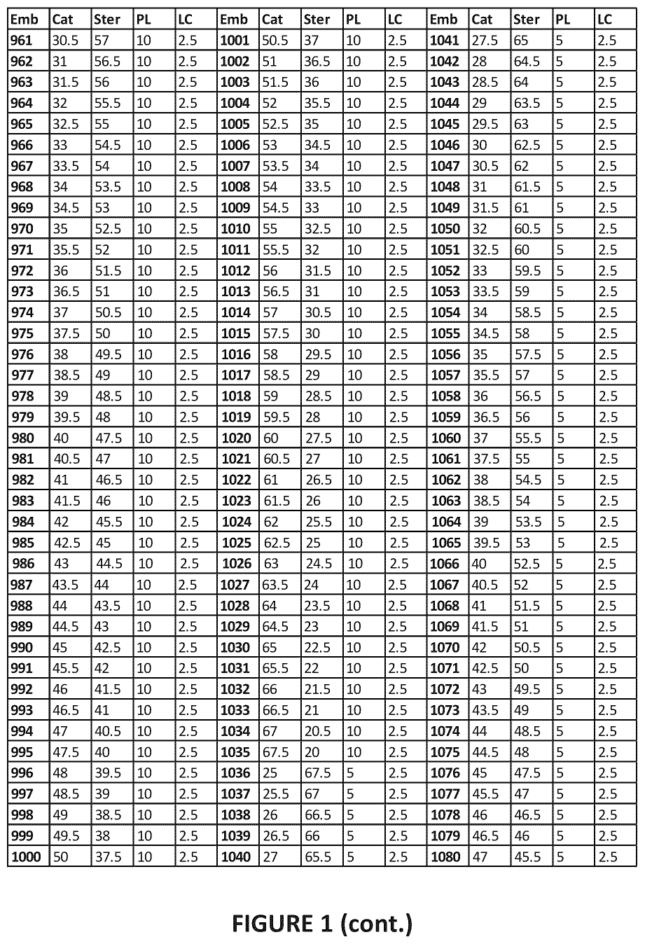

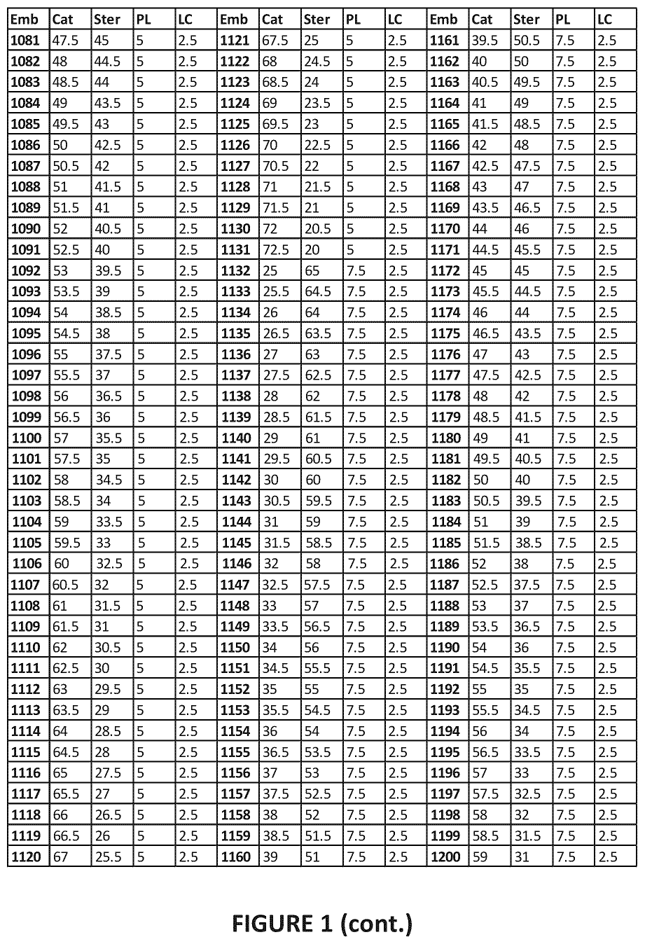

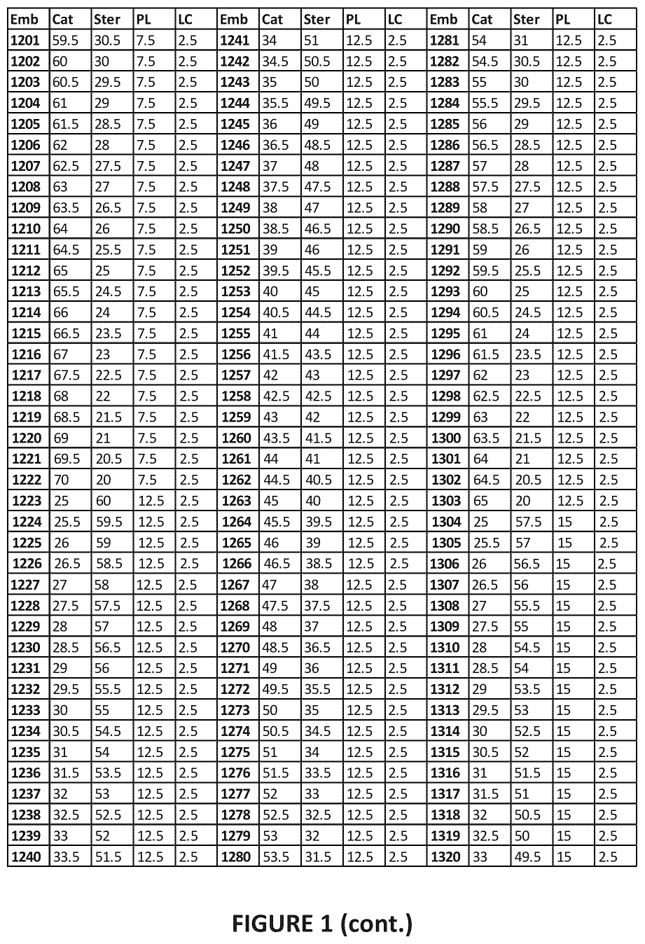

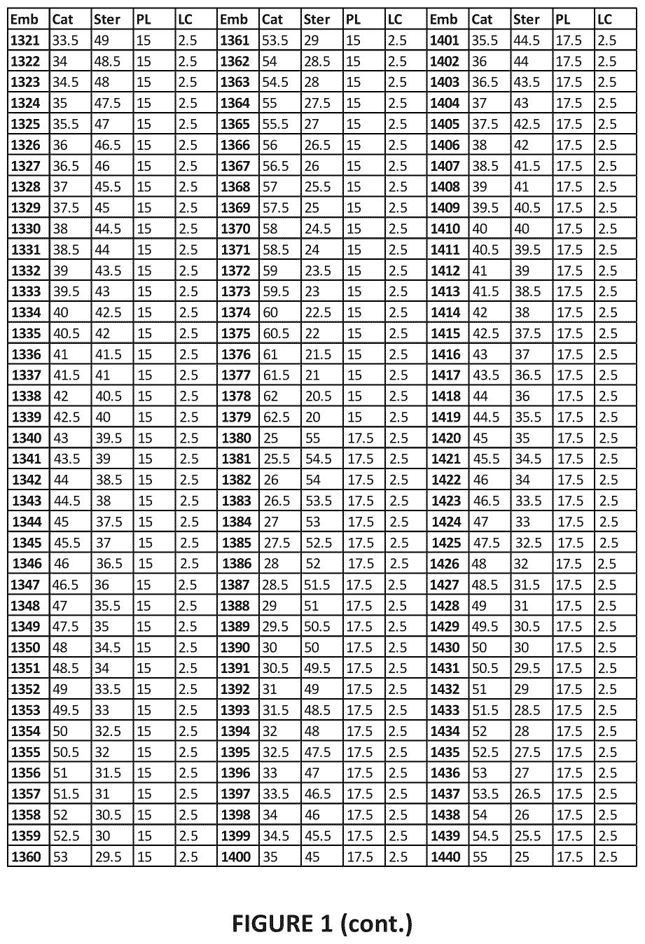

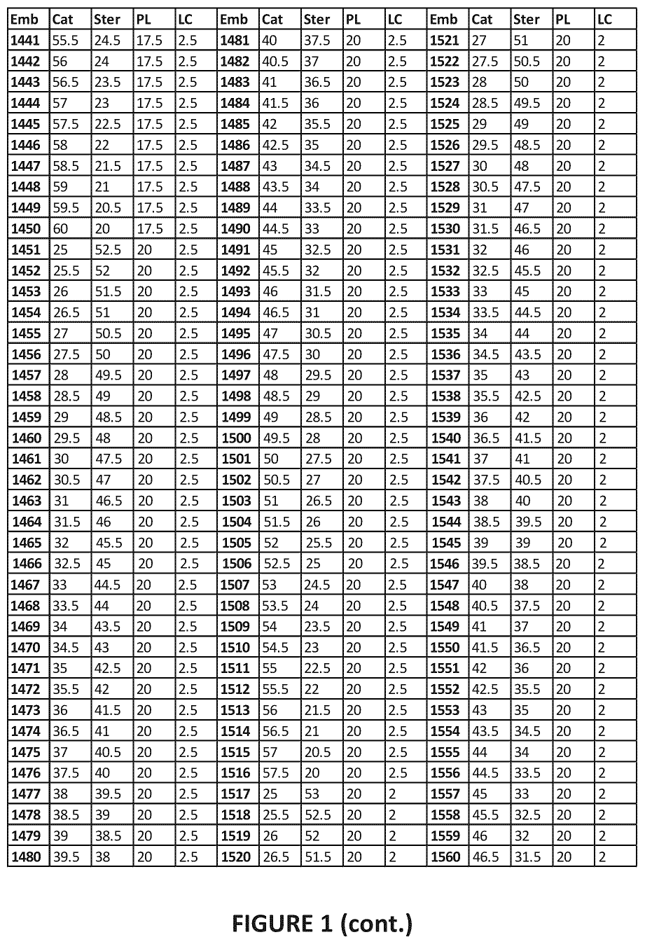

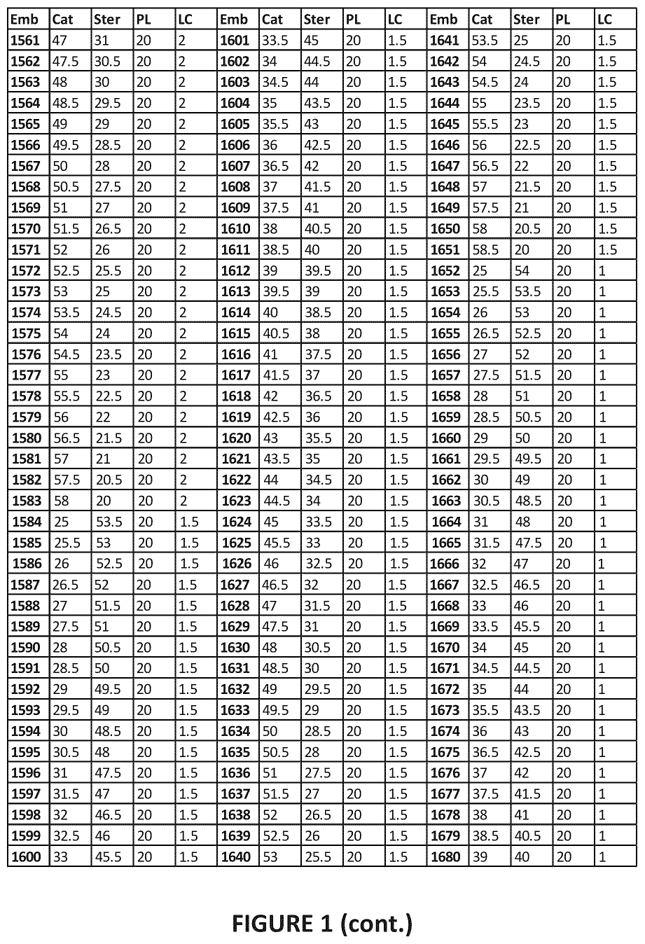

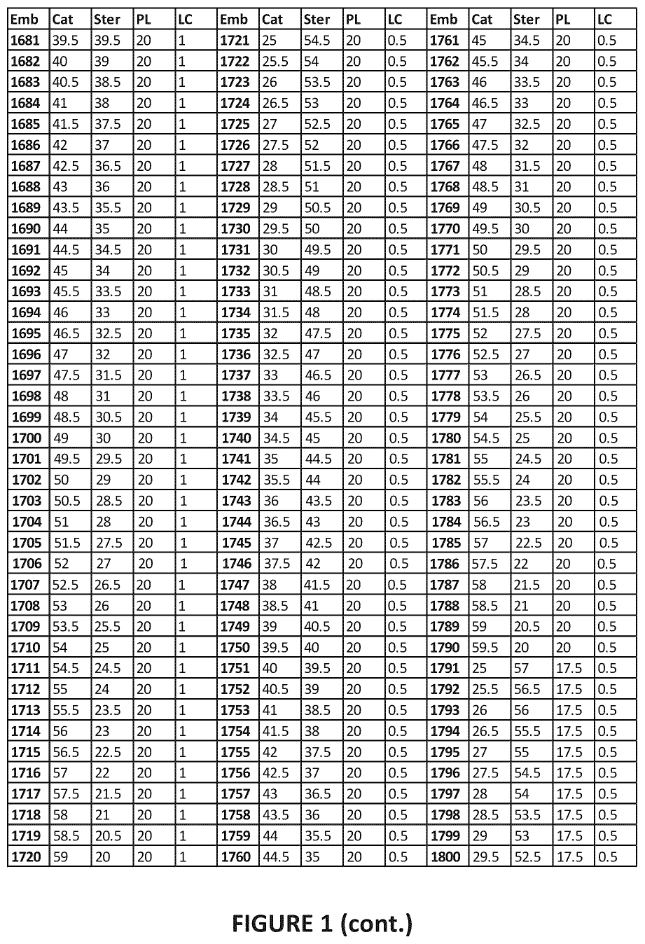

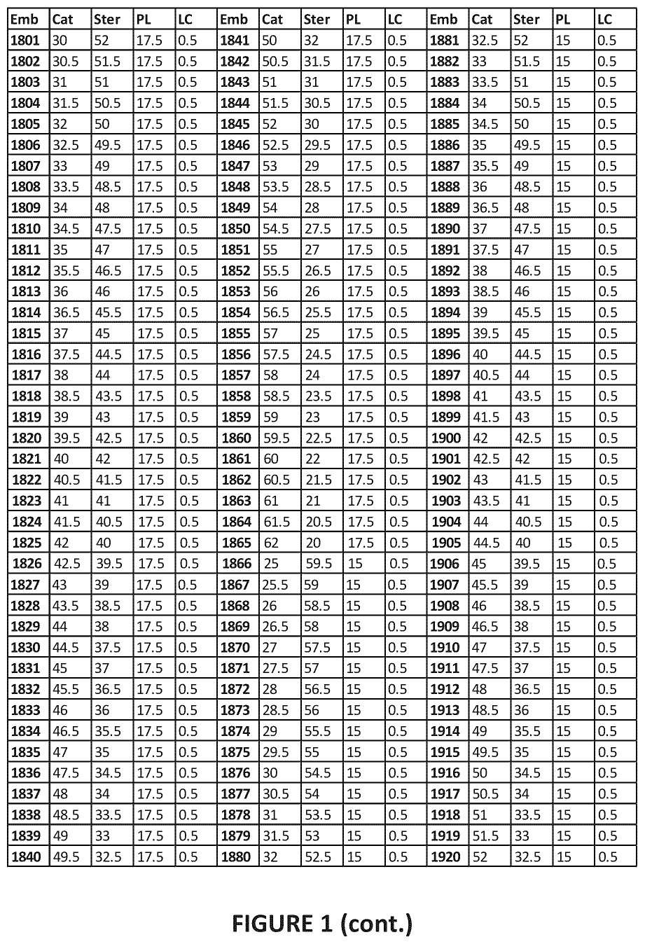

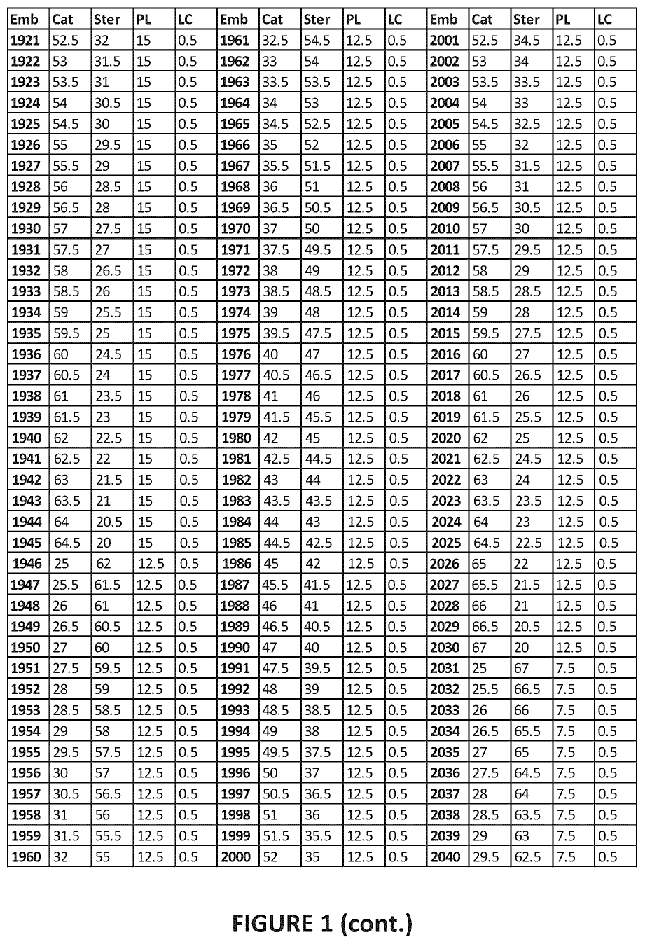

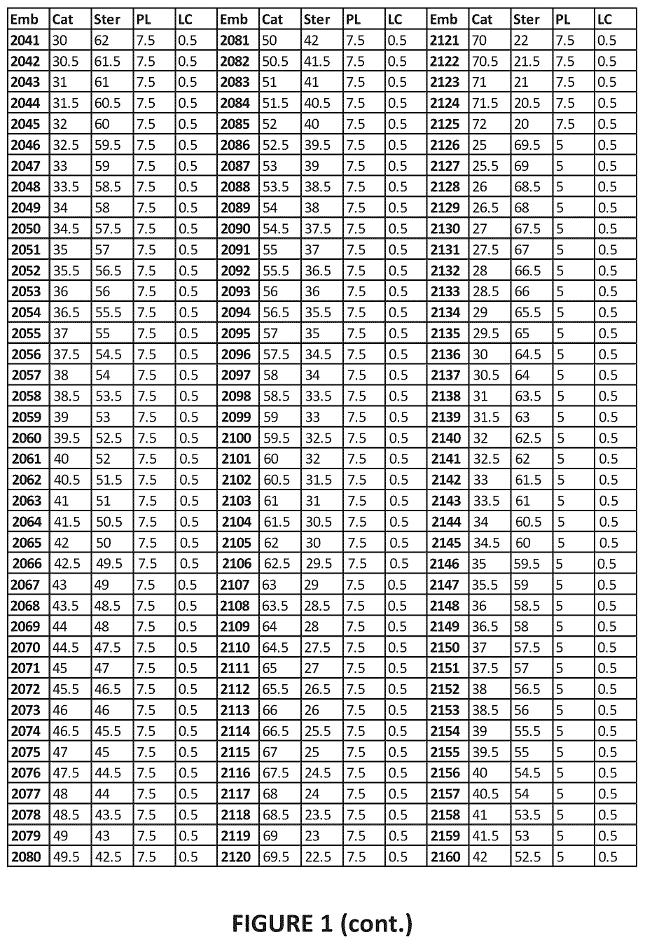

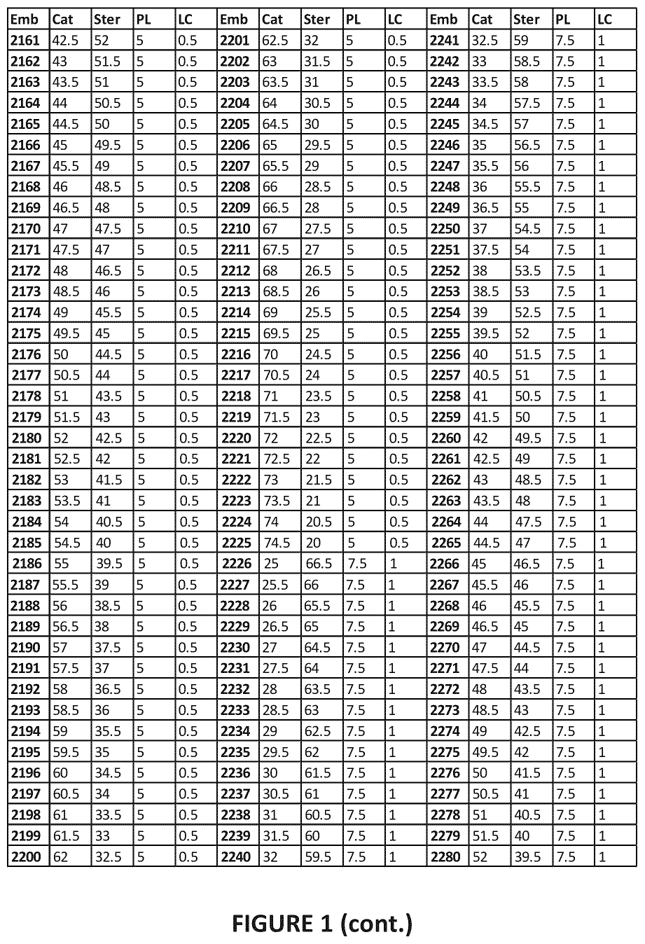

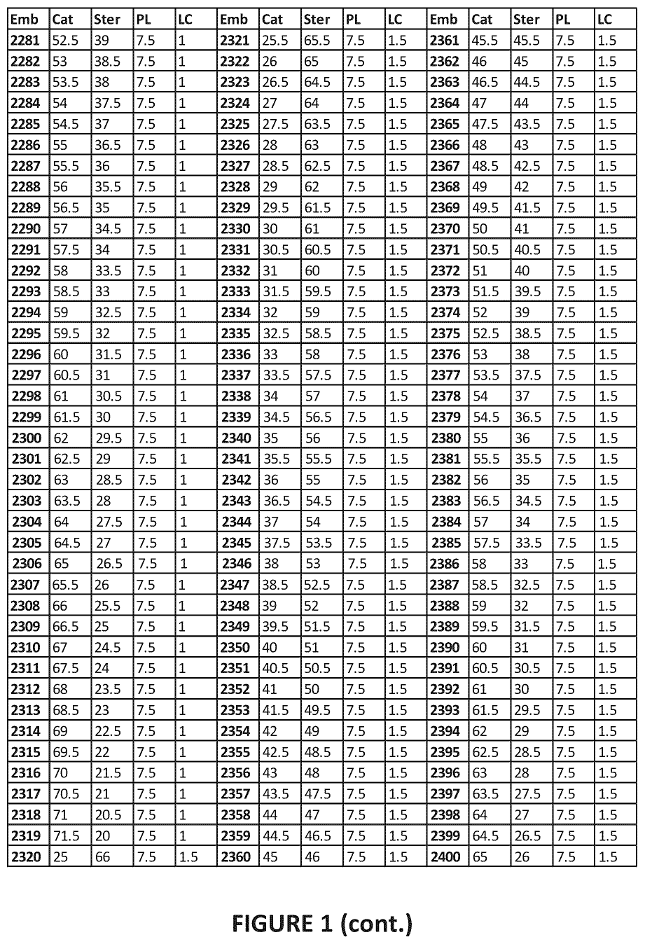

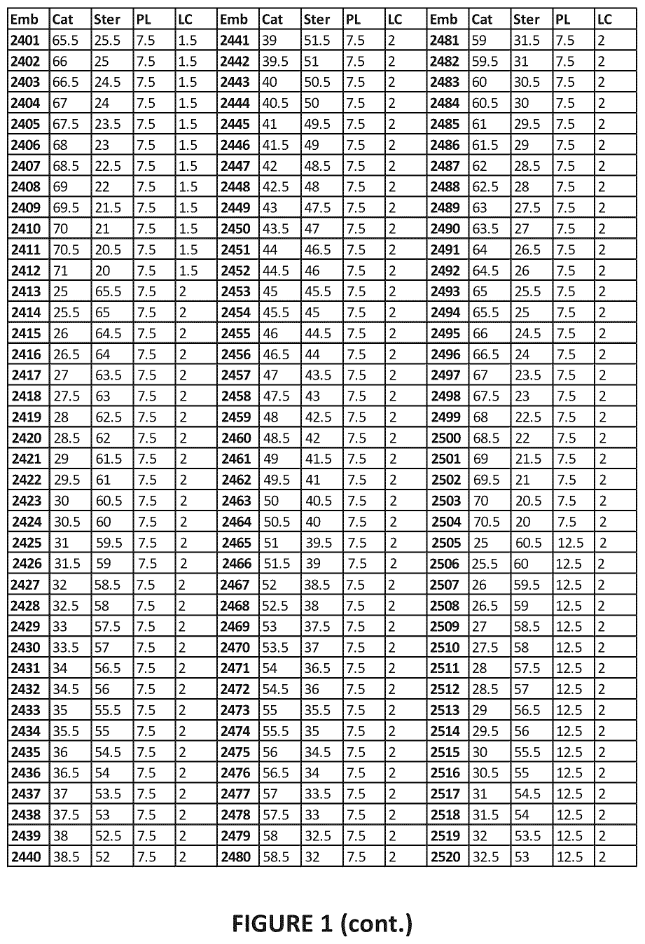

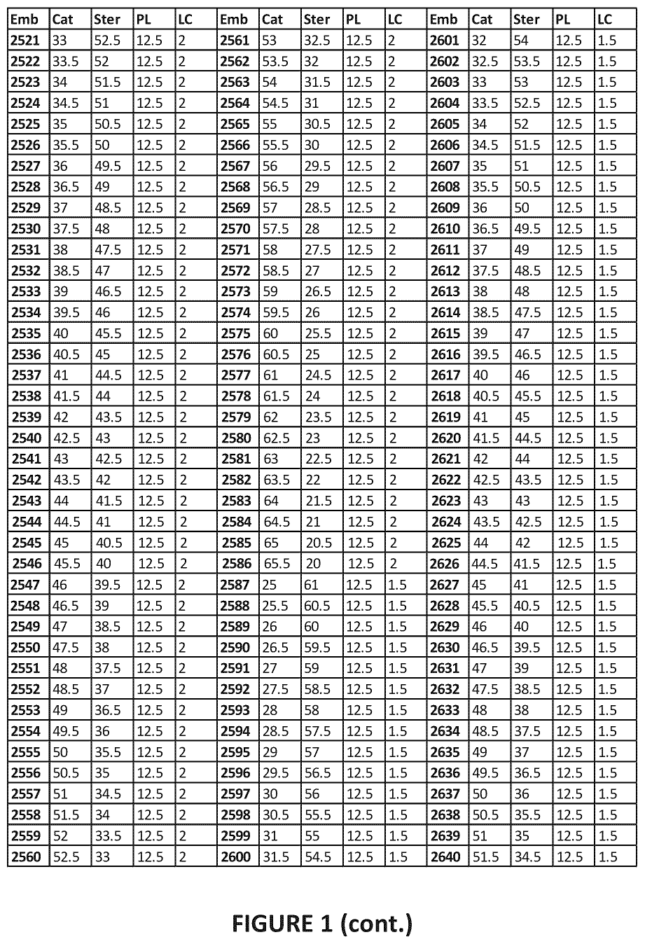

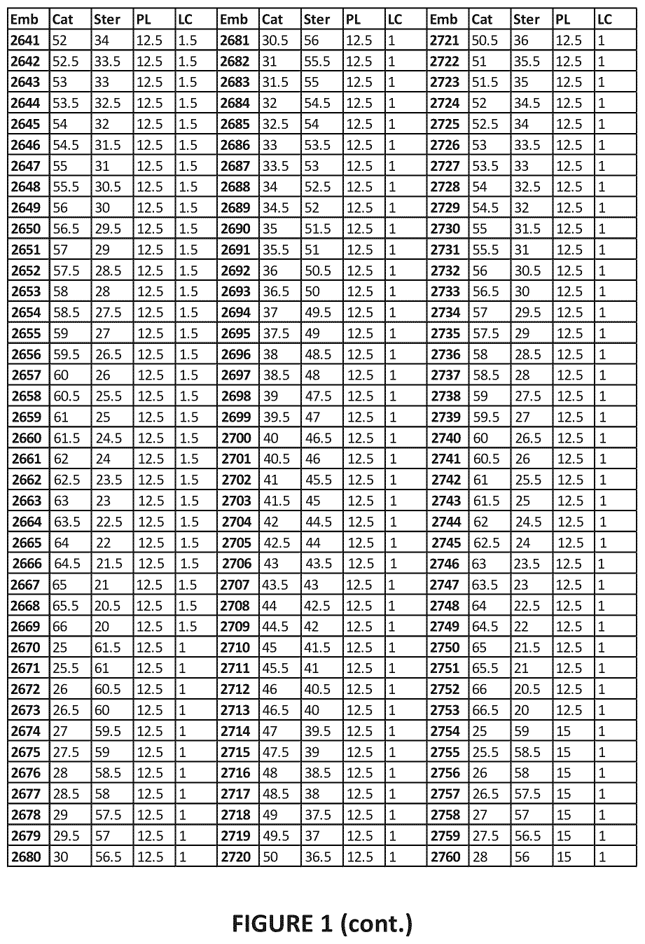

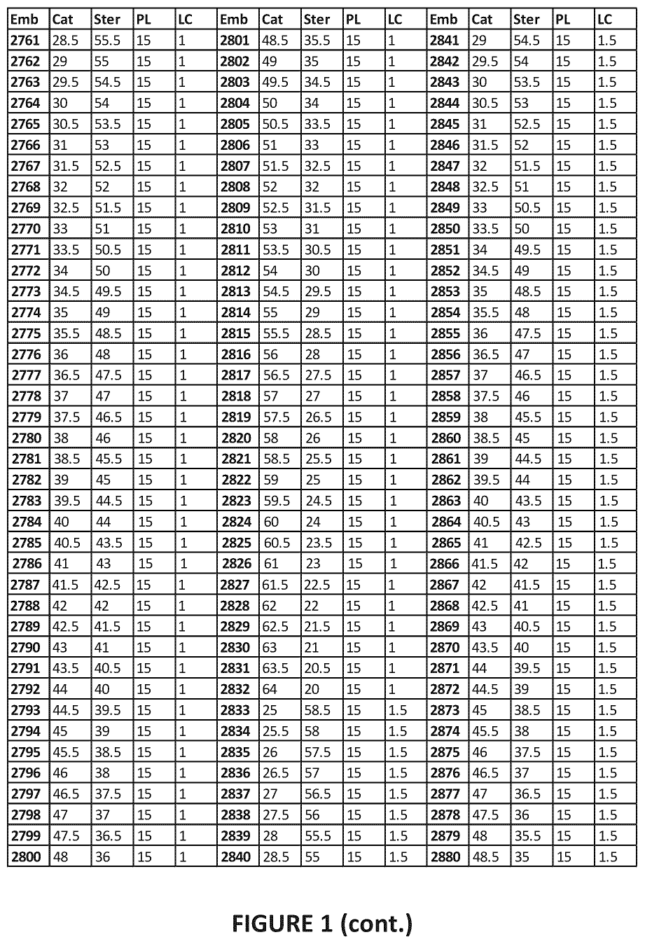

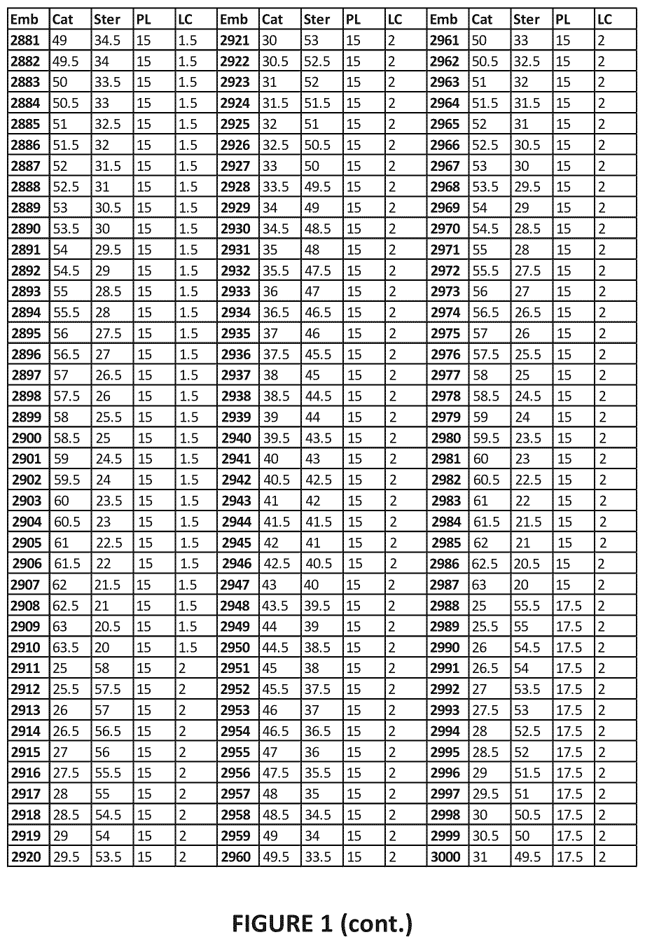

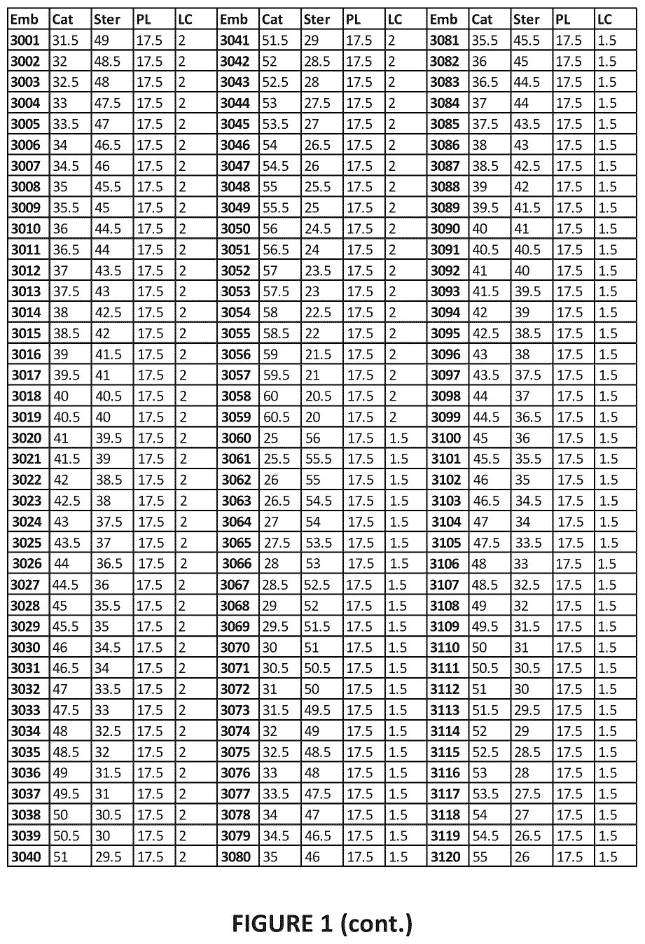

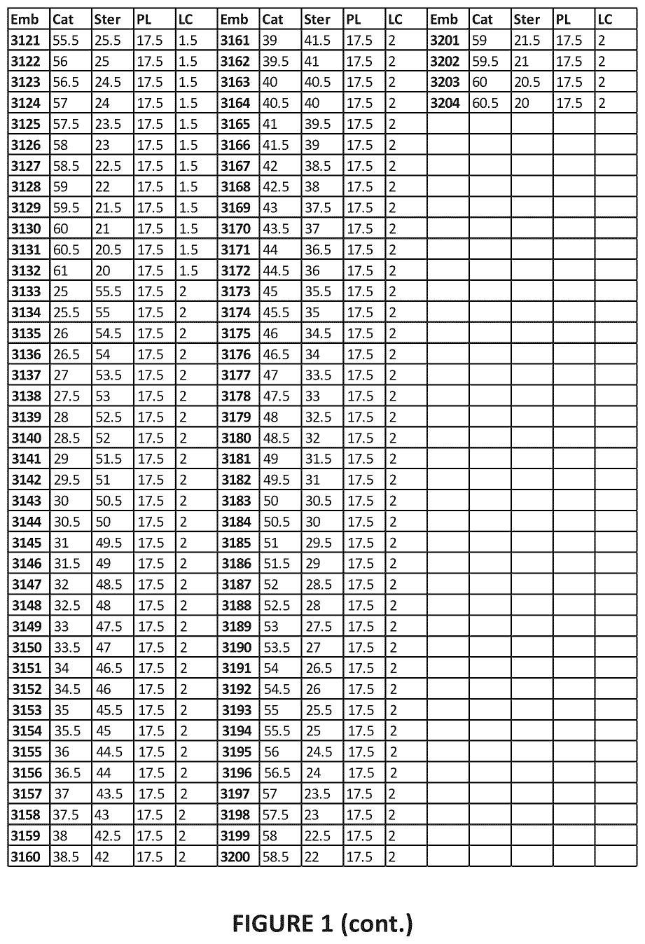

The lipid nanoparticle compositions described herein include one or more cationic lipids, one or more non-cationic lipids (e.g., phospholipids and steroids), and one or more lipid conjugates, wherein an apolipoprotein is bound to at least one lipid component of the lipid nanoparticle. Molar concentrations of each class of lipids are described elsewhere herein, and in the table of FIG. 1, for exemplary lipid nanoparticles embodied by the invention. Also described elsewhere herein are particular combinations of lipids from each class of lipids for exemplary lipid nanoparticles embodied by the invention. Further described elsewhere herein are particular ratios of each lipid component to other lipid components in exemplary lipid nanoparticles embodied by the invention. Further still described elsewhere herein are particular configurations (i.e., type of bond, lipid bound to, presence of modified and unmodified lipids) for apolipoproteins bound to a lipid component in exemplary lipid nanoparticles embodied by the invention.

The selection of cationic lipids, non-cationic lipids, and lipid conjugates which comprise the lipid nanoparticle, as well as the relative molar ratio of such lipids to each other, is based upon the characteristics of the selected lipid(s), the nature of the intended target cells, and the characteristics of the payload (e.g., nucleic acid) to be delivered. Additional considerations include, for example, the saturation of the alkyl chain, as well as the size, charge, pH, pKa, fusogenicity and toxicity of the selected lipid(s). Thus, the molar ratios of each individual component may be adjusted accordingly.

The lipid nanoparticles for use in the method of the invention can be prepared by various techniques which are presently known in the art. Nucleic acid-lipid particles and their method of preparation are disclosed in, for example, U.S. Patent Publication Nos. 2004/0142025 and 2007/0042031, the disclosures of which are herein incorporated by reference in their entirety for all purposes.

Selection of the appropriate size of lipid nanoparticles must take into consideration the site of the target cell and the application for which the lipid nanoparticles is being made. Generally, the lipid nanoparticles will have a size within the range of about 25 to about 500 nm. In some embodiments, the lipid nanoparticles have a size from about 50 nm to about 300 nm, or from about 60 nm to about 120 nm. The size of the lipid nanoparticles may be determined by quasi-electric light scattering (QELS) as described in Bloomfield, Ann. Rev. Biophys. Bioeng., 10:421\hat{ }150 (1981), incorporated herein by reference. A variety of methods are known in the art for producing a population of lipid nanoparticles of particular size ranges, for example, sonication or homogenization. One such method is described in U.S. Pat. No. 4,737,323, incorporated herein by reference.

In some embodiments, the polydispersity index of the lipid nanoparticles is less than about 0.3, or less than about 0.2.

In some embodiments, the zeta potential of the lipid nanoparticles is from about −40 mV to about 40 mV, or from about −10 mV to about 10 mV.

### Chemical Bonds to Apolipoproteins and Chemical Modification of Lipids

The lipid nanoparticles described herein are bound to an apolipoprotein. Various types of bonds can be used to associate the apolipoprotein with at least one lipid component of the lipid nanoparticles. In some embodiments, the apolipoprotein is bound to at least one lipid by hydrophobic bonds. In certain embodiments, the apolipoprotein is bound to at least one lipid by hydrophilic bonds. In certain embodiments, the apolipoprotein is bound to at least one lipid by noncovalent bonds.

In particular embodiments, the apolipoprotein is bound to at least one lipid by a covalent bond. In order to enable a covalent bond to an apolipoprotein, the lipid component can be chemically modified to introduce a functional group capable of forming such a covalent bond. In particular examples, the chemical modification can be at the terminus of the lipid, such that the apolipoprotein is covalently bound to the terminus of the lipid.

Various types of chemical modifications can be made to the lipids of the lipid nanoparticle to facilitate a covalent bond. Chemical modifications to a lipid of the lipid nanoparticle can include, without limitation, the introduction of an amino group, a carboxyl group, a hydroxyl group, a sulfhydryl group, a maleimide group, an acyl halide group, an acetyl halide group, an aldehyde group, an azo group, an azide group, an alkyne group, an alkene group, a haloalkane group, a phosphine group, an imine group, a thiol group, a sulfoxide group, a sulfone group, a sulfonic acid group, a sulfide group, a peroxide group, a chelating group, an ester group, an epoxide group, a nitrone group, a cyclooctynes group, a sulfonyl halides group, a β-Propiolactone group, a γ-butyrolactone group, a β-lactam group, a boronic acid group, or an aryl urea with one nitrogen being in aliphatic ring group.

The covalent bond between the apolipoprotein can include various types of covalent bonds including, but not limited to, an amide bond, a thioester bond, a disulfide bond, a hydrazone bond, an imine bond, an azole bond, and a triazole bond.

In lipid nanoparticles comprising a chemically modified lipid that binds the apolipoprotein, the lipid nanoparticle can further comprise an unmodified lipid of the same class (i.e., cationic, non-cationic, lipid conjugate). Indeed, it may be beneficial for only a portion of the lipids of a particular class to be bound to the apolipoprotein in order to regulate the number of apolipoproteins associated with the lipid nanoparticle. By way of example, the one or more cationic lipids of a lipid nanoparticle described herein can include both an unmodified cationic lipid and a chemically modified cationic lipid that is covalently bound to the apolipoprotein via a functional group. Similarly, the one or more non-cationic lipids can include both an unmodified non-cationic lipid (e.g., an unmodified phospholipid or steroid) and a chemically modified non-cationic lipid (e.g., a modified phospholipid or steroid) that is covalently bound to the apolipoprotein via a functional group. Likewise, the one or more lipid conjugates can include both an unmodified lipid conjugate and a chemically modified lipid conjugate that is covalently bound to the apolipoprotein via a functional group.

In some cases where a certain class of lipids includes both a chemically modified lipid and an unmodified lipid, the chemically modified lipid can be derived from the unmodified lipid. By way of example, the one or more lipid conjugates of a lipid nanoparticle may include an unmodified DSPE-PEG2000 lipid and a modified DSPE-PEG2000 lipid that includes a functionalized group capable of forming a covalent bond with an apolipoprotein. In other cases, the chemically modified lipid can be derived from a lipid other than the unmodified lipid. By way of example, a lipid nanoparticle may comprise an unmodified DMG-PEG5000 lipid and a modified DSPE-PEG2000 lipid that includes a functional group capable of forming a covalent bond with an apolipoprotein.

In cases where a class of lipids includes both a modified lipid and an unmodified lipid, it should be understood that the total molar concentration of that class of lipids represents the total molar amount of both the chemically modified lipid and the unmodified lipid. By way of example, in a case wherein the total molar concentration of the one or more lipid conjugates is 1.5% of the total lipid molar concentration, the 1.5% represents the cumulative amount of both the chemically modified lipid and the unmodified lipid.

It should be further understood that the total molar concentration of a class of lipids can be subdivided into the ratio of the unmodified lipid to the chemically modified lipid. By way of example, a lipid nanoparticle composition may have a total molar concentration of the one or more lipid conjugates of about 1.5%, wherein the one or more lipid conjugates includes an unmodified DMG-PEG5000 lipid and a chemically modified DSPE-PEG2000 lipid that includes a functional group capable of forming a covalent bond with an apolipoprotein. In such cases, various ratios of the chemically modified lipid to the unmodified lipid can constitute the total molar concentration of that class of lipids. Continuing with the above example, the ratio of the chemically modified DSPE-PEG2000 lipid to the unmodified DMG-PEG5000 lipid could be about 1:150, with the total molar concentration of the lipid conjugates representing 1.5% of the total lipid molar concentration of the lipid nanoparticle. Such ratios of the chemically modified lipid to the unmodified lipid can be modified to regulate the total amount of apolipoprotein bound to the lipid nanoparticle. Particular ratios of a chemically modified lipid to an unmodified lipid, that can comprise the total molar concentration of the one or more cationic lipids, the one or more non-cationic lipids (e.g., phospholipids or steroids), or the one or more lipid conjugates, are disclosed elsewhere herein.

### Biological Targeting Molecules

Given the efficiency of the presently disclosed lipid nanoparticles for delivering a payload into a eukaryotic cell (e.g., a T cell or NK cell), cell targeting molecules (e.g., T cell or NK cell targeting molecules) on the surface of the lipid nanoparticles may not be necessary. Thus, in some embodiments, the lipid nanoparticles do not comprise a biological targeting molecule such as, for example, a targeting ligand (e.g., antibodies, scFv proteins, DART molecules, peptides, aptamers, and the like) anchored on the surface of the lipid nanoparticle that selectively binds the lipid nanoparticles to target cells.

### 2.3 Apolipoproteins

According to the present disclosure, it has surprisingly been found that the transfection of eukaryotic cells with lipid nanoparticles, and the delivery of encapsulated payloads (e.g., nucleic acids), can be enhanced by the presence of apolipoproteins (e.g., ApoE) bound to a lipid component of the lipid nanoparticle, particularly in the presence of high concentrations of serum in culture medium. Such high serum concentrations can be optimal for the culture of primary eukaryotic cells, such as primary immune cells (e.g., T cells and NK cells). Apolipoproteins are proteins that bind to and assist in solubilizing hydrophobic lipids and aiding in their transport. Apolipoproteins possess amphipathic (detergent-like) properties and surround hydrophobic lipids to create a lipoprotein particle that is water soluble. Apolipoproteins are components of different lipoproteins and can be defined as non-exchangeable or exchangeable. For example, ApoB is non-exchangeable and anchored in the lipoprotein particle, whereas apoA1, ApoE, ApoD, ApoJ, ApoH, and ApoM are exchangeable and can be transferred between different lipoprotein particles.

In some embodiments, the apolipoprotein used in the presently disclosed methods is an apolipoprotein A (ApoA), apolipoprotein B (ApoB), apolipoprotein C (ApoC), apolipoprotein D (ApoD), apolipoprotein E (ApoE), apolipoprotein H (ApoH), apolipoprotein L (ApoL), apolipoprotein M (ApoM), apolipoprotein (a) (Apo(a)) protein, or a combination thereof. In some of these embodiments, the apolipoprotein is ApoE. ApoE can be any isoform of ApoE, including, for example, ApoE2, ApoE3, and ApoE4.

In examples wherein the apolipoprotein (e.g., ApoE) is covalently bound to a lipid component of the lipid nanoparticle, such lipid nanoparticles can be manufactured by methods known in the art and, for example, those disclosed herein where the lipid nanoparticles are first manufactured and then subsequently contacted with the apolipoprotein in order to induce a covalent bond to the modified lipid.

### 2.4 Introduction of Nucleic Acids into Eukaryotic Cells

### Eukaryotic Cells

As previously described herein, the present disclosure generally provides lipid nanoparticle compositions, and methods of using the same, which are useful for transfecting eukaryotic cells and introducing an encapsulated payload (e.g., a nucleic acid).

In some examples of the invention, the eukaryotic cells are human immune cells. In some embodiments, the human immune cells are T cells, or cells derived therefrom. In other embodiments, the human immune cells are natural killer (NK) cells, or cells derived therefrom. In still other embodiments, the immune cells are human B cells, or cells derived therefrom. In still other embodiments, the human immune cells are macrophages, or cells derived therefrom. In other examples of the invention, the eukaryotic cells are human induced pluripotent stem cells (iPSCs).

Immune cells can be obtained from a number of sources, including peripheral blood mononuclear cells, bone marrow, lymph node tissue, cord blood, thymus tissue, tissue from a site of infection, ascites, pleural effusion, spleen tissue, and tumors. In some embodiments of the present disclosure, immune cells are obtained from a unit of blood collected from a subject using any number of techniques known to the skilled artisan. In one embodiment, cells from the circulating blood of an individual are obtained by apheresis.

### Culture Conditions

In some embodiments, the lipid nanoparticle composition is contacted with the eukaryotic cells (e.g., human immune cells) under in vitro (i.e., cell culture) conditions.

In some such cases, the cells are contacted with the lipid nanoparticles in a cell culture condition wherein the medium comprises a concentration of serum (vol/vol) of between about 1% and about 25%. In some embodiments, the cells are contacted with the lipid nanoparticles in a cell culture condition wherein the medium comprises a concentration of serum (vol/vol) of between about 2.5% and about 20%. In some embodiments, the cells are contacted with the lipid nanoparticles in a cell culture condition wherein the medium comprises a concentration of serum (vol/vol) of between about 5% and about 20%. In some embodiments, the cells are contacted with the lipid nanoparticles in a cell culture condition wherein the medium comprises a concentration of serum (vol/vol) of between about 10% and about 20%. In some embodiments, the cells are contacted with the lipid nanoparticles in a cell culture condition wherein the medium comprises a concentration of serum (vol/vol) of between about 15% and about 20%. In some embodiments, the cells are contacted with the lipid nanoparticles in a cell culture condition wherein the medium comprises a concentration of serum (vol/vol) of about 5%. In some embodiments, the cells are contacted with the lipid nanoparticles in a cell culture condition wherein the medium comprises a concentration of serum (vol/vol) of about 10%. In some embodiments, the cells are contacted with the lipid nanoparticles in a cell culture condition wherein the medium comprises a concentration of serum (vol/vol) of about 15%. In some embodiments, the cells are contacted with the lipid nanoparticles in a cell culture condition wherein the medium comprises a concentration of serum (vol/vol) of about 20%. In some embodiments, the cells are contacted with the lipid nanoparticles in a cell culture condition wherein the medium comprises a concentration of serum (vol/vol) of about 25%.

In examples wherein the eukaryotic cells described herein are immune cells, such immune cells (e.g., T cells or NK cells) may require activation prior to contacting the cells with the lipid nanoparticles and/or introduction of a donor template. For example, T cells can be contacted with anti-CD3 and anti-CD28 antibodies that are soluble or conjugated to a support (i.e., beads) for a period of time sufficient to activate the cells.

Some standard methods of introducing nucleic acids into eukaryotic cells (e.g., mRNA encoding an engineered nuclease) utilize electroporation to enhance cellular permeability and allow penetration of the nucleic acid into the cell. The process of electroporation requires that cells be removed from their vessel, centrifuged, re-suspended in specific buffers, and moved to new vessels. The introduction of a nucleic acid comprising a donor template can require further isolation and movement of cells if different media conditions are required. By comparison, the methods disclosed herein allow for a greatly simplified process by which a nucleic acid (e.g., an mRNA encoding a nuclease) can be introduced into eukaryotic cells in combination with a donor template (e.g., encoding a polypeptide of interest, such as CAR or exogenous TCR). In some embodiments, the eukaryotic cells are not transferred to a new vessel between the step of contacting the cells with the lipid nanoparticles described herein and the introduction of the donor template. In some embodiments, the cells are not centrifuged between the step of contacting the cells with the lipid nanoparticles and the step of introducing the donor template. For example, in particular embodiments, the cells can be contacted with lipid nanoparticles described herein and an AAV comprising the donor template in the same vessel, avoiding the need for centrifugation, re-suspension, and movement between multiple vessels.

### Nucleic Acids

In various examples, the lipid nanoparticles described herein comprise a nucleic acid. A variety of nucleic acids can be encapsulated as payloads. In some examples, the nucleic acid is an mRNA. In some examples, the nucleic acid is a DNA molecule. In certain examples, the DNA molecule is a double-stranded DNA (dsDNA). In certain examples, the DNA molecule is a recombinant DNA construct. In other embodiments, the DNA molecule is a single-stranded DNA (ssDNA). In some examples, the nucleic acid is an RNA interference (RNAi) molecule. In yet other examples, the nucleic acid is a mixture of RNAs. In further examples, the nucleic acid is a mixture of RNA and DNA. In some examples, the nucleic acid is double-stranded (dsRNA). In certain examples, the nucleic acid is an aptamer, an adjuvant, or a decoy.

### Introduction of Nucleic Acids Encoding Engineered Nucleases

The lipid nanoparticles described herein can comprise, for example, a nucleic acid encoding an engineered nuclease having specificity for a recognition sequence in the genome of a eukaryotic cell.

In some examples, the nucleic acid encoding the engineered nucleases is an mRNA. In other examples, the nucleic acid encoding the engineered nuclease is a DNA (e.g., a dsDNA).

Following contact of the lipid nanoparticles with the cell, the nucleic acid is delivered into the cells and the engineered nuclease is expressed. Upon expression, the engineered nuclease subsequently generates a cleavage site at its recognition sequence. Such recognition sequences may be in a target gene. In some examples, a cleavage site in a target gene is repaired by error-prone non-homologous end joining, resulting in disrupted expression of the polypeptide encoded by the gene. In some other examples, an exogenous polynucleotide is inserted into the cleavage site. In some such cases, insertion of the donor template results in disrupted expression of the polypeptide encoded by the gene. If the donor template comprises a transgene, such a transgene may be expressed.

In some embodiments, the engineered nuclease encoded by the nucleic acid, and which generates the cleavage site in the eukaryotic cell genome, is an engineered meganuclease, a zinc finger nuclease, a TALEN, a compact TALEN, a CRISPR system nuclease, or a megaTAL. In certain embodiments, the engineered nuclease is an engineered meganuclease. In particular embodiments, the engineered nuclease used to practice the invention is a single-chain meganuclease.

In some embodiments, the recognition sequence of the engineered nuclease is in a target gene. In particular embodiments, the target gene can be a TCR alpha gene, a TCR alpha constant region (TRAC) gene, a TCR beta gene, a TCR beta constant region (TRBC) gene, a beta-2 microglobulin gene, a CD52 gene, a CS1 (i.e., SLAMF7 or CD319) gene, a Cbl proto-oncogene B (CBL-B) gene, a CD52 gene, a CD7 gene, a programmed cell death −1 (PD-1) gene, a lymphocyte-activation 3 (LAG-3) gene, a transforming growth factor beta receptor II (TGFBRII) gene, a T-cell immunoglobulin and mucin-domain containing-3 (TIM-3) gene, a T cell immunoreceptor with Ig and ITIM domains (TIGIT) gene, a CD70 gene, a glucocorticoid receptor gene, a Tet methylcytosine dioxygenase 2 (TET2) gene, a general control nonderepressible 2 (GCN2) gene, a deoxycytidine kinase (DCK) gene, a cytotoxic T-lymphocyte associated protein 4 (CTLA-4) gene, or a C-C motif chemokine receptor 5 (CCR5) gene.

In particular embodiments, the target gene is a TCR alpha gene. In other embodiments, the target gene is a TRAC gene. In some embodiments, the target gene is a TCR beta gene. In certain embodiments, the target gene is a TRBC gene. In such embodiments wherein the target gene encodes a component of the endogenous alpha/beta TCR, eukaryotic cells (e.g., T cells or NK cells) prepared according to the disclosed methods do not have detectable cell-surface expression of an endogenous alpha/beta TCR. In particular embodiments, the genetically-modified immune cells express a CAR or exogenous TCR.

In other embodiments, the engineered nuclease has specificity for a recognition sequence located within a safe harbor locus. As used herein, the phrase “safe harbor locus” refers to chromosomal loci where exogenous nucleic acid inserts can be stably and reliably expressed in all tissues of interest without overtly altering cell behavior or phenotype (i.e., without any deleterious effects on the host cell).

In particular embodiments, eukaryotic cells (e.g., T cells or NK cells) can be contacted with a first population of lipid nanoparticles comprising a nucleic acid encoding a first engineered nuclease having specificity for a first recognition sequence, and simultaneously or subsequently contacted with a second population of lipid nanoparticles comprising a nucleic acid encoding a second engineered nuclease having specificity for a second recognition sequence. In such embodiments of the methods, the first engineered nuclease and the second engineered nuclease are expressed in the cells, the first engineered nuclease generates a first cleavage site in the first recognition sequence, and the second engineered nuclease generates a second cleavage site in the second recognition sequence. In some instances, the first recognition sequence and the second recognition sequence are in the same target gene, such that expression of a polypeptide encoded by the target gene is disrupted by non-homologous end joining or insertion of a donor template at the first cleavage site and/or the second cleavage site. In other examples, the first recognition sequence and the second recognition sequence are in different target genes, such that expression of polypeptides encoded by the different target genes is disrupted by non-homologous end joining or insertion of a donor template at the first cleavage site and the second cleavage site. The target gene(s) targeted by these methods can be any target gene(s) of interest, including those previously discussed herein. In some examples, where a single target gene is disrupted, the target gene can be the TRAC gene. In some examples where two target genes are disrupted, the target genes can be the TRAC gene and the beta-2 microglobulin gene.

Donor templates that are introduced into eukaryotic cells, and are inserted into the nuclease cleavage sites, can comprise a 5′ homology arm and a 3′ homology aim flanking the elements of the insert. Such homology arms have sequence homology to corresponding sequences 5′ upstream and 3′ downstream of the nuclease recognition sequence where a cleavage site is produced. In general, homology arms can have a length of at least 50 base pairs, preferably at least 100 base pairs, and up to 2000 base pairs or more, and can have at least 90%, preferably at least 95%, or more, sequence homology to their corresponding sequences in the genome.

In various embodiments, the donor template can comprise a coding sequence for a polypeptide of interest. It is envisioned that the coding sequence can be for any polypeptide of interest. In particular embodiments of the method, the polypeptide of interest can be a CAR or an exogenous TCR. In still other embodiments, the donor template can encode the wild-type or modified version of an endogenous gene of interest.

A donor template described herein can further comprise additional control sequences. For example, the donor template can include homologous recombination enhancer sequences, Kozak sequences, polyadenylation sequences, transcriptional termination sequences, selectable marker sequences (e.g., antibiotic resistance genes), origins of replication, and the like. A donor template described herein can also include at least one nuclear localization signal. Examples of nuclear localization signals are known in the art (see, e.g., Lange et al., J. Biol. Chem., 2007, 282:5101-5105).

The donor template acid can be introduced into the eukaryotic cells via any method known in the art for delivery of nucleic acids into cells. For embodiments in which the donor template is delivered in DNA form and encodes a polypeptide of interest, the nucleic acid sequence encoding the polypeptide of interest can be operably linked to a promoter to facilitate transcription of the polypeptide. Mammalian promoters suitable for the invention include constitutive promoters such as the cytomegalovirus early (CMV) promoter (Thomsen et al. (1984), Proc Natl Acad Sci USA. 81(3):659-63) or the SV40 early promoter (Benoist and Chambon (1981), Nature. 290(5804):304-10) as well as inducible promoters such as the tetracycline-inducible promoter (Dingermann et al. (1992), Mol Cell Biol. 12(9):4038-45). The coding sequence for the polypeptide of interest can also be operably linked to a synthetic promoter. Synthetic promoters can include, without limitation, the JeT promoter (WO 2002/012514).

In another particular embodiment, the donor template is a single-stranded DNA template. The single-stranded DNA can further comprise a 5′ and/or a 3′ AAV inverted terminal repeat (ITR) upstream and/or downstream of the sequence encoding the polypeptide of interest. In other embodiments, the single-stranded DNA can further comprise a 5′ and/or a 3′ homology arm upstream and/or downstream of the sequence encoding the polypeptide of interest.

In yet another particular embodiment, the donor template is a linearized DNA template. In some examples, a plasmid DNA encoding a polypeptide of interest can be digested by one or more restriction enzymes such that the circular plasmid DNA is linearized prior to being introduced into a cell.

In some embodiments, the donor template is introduced into the cells using a recombinant DNA construct. In some embodiments, the recombinant DNA construct is encapsulated in a lipid nanoparticle. In some embodiments, the recombinant DNA construct is encapsulated in the same lipid nanoparticle that comprises the nucleic acid encoding the engineered nuclease.

In certain embodiments, the donor template is introduced into the eukaryotic cells using a recombinant virus. Such viruses are known in the art and include recombinant retroviruses, recombinant lentiviruses, recombinant adenoviruses, and recombinant adeno-associated viruses (AAVs) (reviewed in Vannucci, et al. (2013 New Microbiol. 36:1-22). Recombinant AAVs useful in the invention can have any serotype that allows for transduction of the virus into the cell. In particular embodiments, when transducing human immune cells such as T cells or NK cells, recombinant AAVs can have a serotype of AAV2 or AAV6. Recombinant AAVs can also be self-complementary such that they do not require second-strand DNA synthesis in the host cell (McCarty, et al. (2001) Gene Ther. 8:1248-54). In particular embodiments, the recombinant vines comprising the donor template is a recombinant AAV.

The donor template can be introduced into cells prior to contacting the cells with the lipid nanoparticles described herein (i.e., comprising the nucleic acid encoding the engineered nuclease), after contacting the cells with the lipid nanoparticles described herein, or simultaneously with contacting the cells with the lipid nanoparticles described herein. In certain examples, the donor template can be introduced into the cells between 0 and about 48 hours, 0 to about 24 hours, or about 24 to about 48 hours, after contacting the cells with the lipid nanoparticles described herein (i.e., comprising the nucleic acid encoding the engineered nuclease). In a particular example, the donor template can be introduced into the cells between 24 and 48 hours after contacting the cells with the lipid nanoparticles described herein (i.e., comprising the nucleic acid encoding the engineered nuclease).

### Introduction of Donor Templates

In other embodiments of the invention, the lipid nanoparticles described herein comprise a nucleic acid, such as a DNA molecule (e.g., a dsDNA) comprising a donor template. A donor template can, in some examples, comprise a nucleic acid sequence encoding a polypeptide of interest. Further, a donor template can be configured for insertion into the genome of a eukaryotic cell.

In certain examples, when introduced into a eukaryotic cell (e.g., a T cell or NK cell), a cleavage site generated by an engineered nuclease can allow for homologous recombination of the donor template directly into that cleavage site in the genome. In some cases, the cleavage site is within a target gene, and the donor template is inserted into the target gene. In some embodiments, the target gene can be any one of the target genes described previously herein. Insertion of the donor template can, in some cases, disrupt expression of a polypeptide encoded by the target gene. For example, in particular embodiments, the target gene is a TRAC gene, and insertion of a donor template into a cleavage site in the TRAC gene results in expression of a polypeptide encoded by the donor template (e.g., a CAR or exogenous TCR), while disrupting expression of the TCR alpha subunit, which subsequently prevents assembly of the endogenous alpha/beta TCR on the cell surface.

In other embodiments, the cleavage site into which the donor template is inserted is located within a safe harbor locus. As used herein, the phrase “safe harbor locus” refers to chromosomal loci where exogenous nucleic acid inserts can be stably and reliably expressed in all tissues of interest without overtly altering cell behavior or phenotype (i.e., without any deleterious effects on the host cell).

In some embodiments, the donor template comprises a 5′ homology arm and a 3′ homology arm flanking the elements of the insert. Such homology arms have sequence homology to corresponding sequences 5′ upstream and 3′ downstream of the nuclease recognition sequence where a cleavage site is produced. In general, homology arms can have a length of at least 50 base pairs, preferably at least 100 base pairs, and up to 2000 base pairs or more, and can have at least 90%, preferably at least 95%, or more, sequence homology to their corresponding sequences in the genome.

In various embodiments, the donor template can comprise a coding sequence for a polypeptide of interest. It is envisioned that the coding sequence can be for any polypeptide of interest. In particular embodiments of the method, the polypeptide of interest can be a CAR or an exogenous TCR. In still other embodiments, the donor template can encode the wild-type or modified version of an endogenous gene of interest.

A donor template described herein can further comprise additional control sequences. For example, the donor template can include homologous recombination enhancer sequences, Kozak sequences, polyadenylation sequences, transcriptional termination sequences, selectable marker sequences (e.g., antibiotic resistance genes), origins of replication, and the like. A donor template described herein can also include at least one nuclear localization signal. Examples of nuclear localization signals are known in the art (see, e.g., Lange et al., J. Biol. Chem., 2007, 282:5101-5105).

For embodiments in which the donor template is in DNA form and encodes a polypeptide of interest, the nucleic acid sequence encoding the polypeptide of interest can be operably linked to a promoter to facilitate transcription of the polypeptide. Mammalian promoters suitable for the invention include constitutive promoters such as the cytomegalovirus early (CMV) promoter (Thomsen et al. (1984), Proc Natl Acad Sci USA. 81(3):659-63) or the SV40 early promoter (Benoist and Chambon (1981), Nature. 290(5804):304-10) as well as inducible promoters such as the tetracycline-inducible promoter (Dingermann et al. (1992), Mol Cell Biol. 12(9):4038-45). The polypeptide coding sequence can also be operably linked to a synthetic promoter. Synthetic promoters can include, without limitation, the JeT promoter (WO 2002/012514).

In another particular embodiment, the donor template can be a single-stranded DNA template. The single-stranded DNA can further comprise a 5′ and/or a 3′ AAV inverted terminal repeat (ITR) upstream and/or downstream of the sequence encoding the polypeptide of interest. In other embodiments, the single-stranded DNA can further comprise a 5′ and/or a 3′ homology arm upstream and/or downstream of the sequence encoding the polypeptide of interest.

In yet another particular embodiment, the donor template can be a linearized DNA template. In some examples, a plasmid DNA encoding a polypeptide of interest can be digested by one or more restriction enzymes such that the circular plasmid DNA is linearized prior to being introduced into a cell.

In some embodiments, the donor template acid is a recombinant DNA construct (e.g., a plasmid DNA).

In cases wherein the donor template is introduced into eukaryotic cells along with introduction of a nucleic acid encoding an engineered nuclease, the donor template can be introduced into the cells prior to introduction of the nucleic acid encoding the engineered nuclease, after introduction of the nucleic acid encoding the engineered nuclease, or simultaneously with introduction of the nucleic acid encoding the engineered nuclease. In certain examples, the donor template can be introduced into the cells between 0 and about 48 hours, 0 to about 24 hours, or about 24 to about 48 hours, after introducing the nucleic acid encoding the engineered nuclease. In a particular example, the donor template can be introduced into the cells between 24 and 48 hours after introducing the nucleic acid encoding the engineered nuclease.

### Introduction of RNAi Molecules

In other embodiments of the invention, the nucleic acid comprised by the lipid nanoparticles described herein is an RNAi molecule capable of reducing the expression of an endogenous protein in a eukaryotic cell (e.g., a T cell or NK cell).

The RNAi interference molecule can be any one of a variety of nucleic acids capable of reducing the expression of endogenous proteins including, but not limited to, a short hairpin RNA (shRNA), a small interfering RNA (siRNA), a hairpin siRNA, a microRNA (miRNA), a precursor miRNA, and an miRNA-adapted shRNA.

RNAi molecules utilized in the invention can be directed to any endogenous protein of interest in a eukaryotic cell (e.g., a T cell or NK cell). In some non-limiting examples, an RNAi molecule is inhibitory against a component of the MHC class I molecule, such as the MHC class I alpha-1 domain, alpha-2 domain, alpha-3 domain, or against beta-2 microglobulin. In particular embodiments, the RNAi molecule is inhibitory against beta-2 microglobulin. In other non-limiting examples, the RNAi molecule is inhibitory against CS1, transforming growth factor-beta receptor 2 (TGFBR2), Cbl proto-oncogene B (CBL-B), CD52, a TCR alpha gene, a TRAC gene, a TCR beta gene, a TRBC gene, CD7, glucocorticoid receptor (GR), deoxycytidine kinase (DCK), nuclear receptor subfamily 2 group F member 6 (NR2F6), cytotoxic T-lymphocyte-associated protein 4 (CTLA-4), or C-C chemokine receptor type 5 (CCR5).

### 2.5 Nuclease mRNA

In some embodiments of the invention, the lipid nanoparticles disclosed herein comprise mRNA. Such mRNA can be produced using methods known in the art such as in vitro transcription. In some embodiments, the mRNA comprises a 5′ cap. Such 5′ caps are known in the art and can include, without limitation, an anti-reverse cap analogs (ARCA) (U.S. Pat. No. 7,074,596), 7-methyl-guanosine, CleanCap® analogs, such as Cap 1 analogs (Trilink; San Diego, CA), or enzymatically capped using, for example, a vaccinia capping enzyme or the like. In some embodiments, the mRNA may be polyadenylated. The mRNA may contain various 5′ and 3′ untranslated sequence elements to enhance expression of the encoded engineered nuclease and/or stability of the mRNA itself. Such elements can include, for example, posttranslational regulatory elements such as a woodchuck hepatitis virus posttranslational regulatory element.

The mRNA may contain modifications of naturally-occurring nucleosides to nucleoside analogs. Any nucleoside analogs known in the art are envisioned for use in the present methods. Such nucleoside analogs can include, for example, those described in U.S. Pat. No. 8,278,036. In particular embodiments, nucleoside modifications can include a modification of uridine to pseudouridine, and/or a modification of uridine to N1-methyl pseudouridine.

### 2.6 Chimeric Antigen Receptors and Exogenous T Cell Receptors

In certain embodiments, the lipid nanoparticles described herein can be used to introduce a donor template into the genome of a eukaryotic cell (e.g., a T cell or NK cell), wherein the donor template comprises a nucleic acid sequence encoding a chimeric antigen receptor (CAR). Generally, a CAR of the present disclosure will comprise at least an extracellular domain, a transmembrane domain, and an intracellular domain. In some embodiments, the extracellular domain comprises a target-specific binding element otherwise referred to as an extracellular ligand-binding domain or moiety. In some embodiments, the intracellular domain, or cytoplasmic domain, comprises at least one co-stimulatory domain and one or more signaling domains.

In some embodiments, a CAR useful in the invention comprises an extracellular ligand-binding domain having specificity for a target cell antigen (i.e., an antigen expressed on the surface of a target cell, such as a cancer cell). The choice of ligand-binding domain depends upon the type and number of ligands that define the surface of a target cell. For example, the ligand-binding domain may be chosen to recognize a ligand that acts as a cell surface marker on target cells associated with a particular disease state. Thus, some examples of cell surface markers that may act as ligands for the ligand-binding domain in a CAR can include those associated cancer cells. In some embodiments, a CAR is engineered to target a cancer-specific antigen of interest by way of engineering a desired ligand-binding moiety that specifically binds to an antigen on a cancer cell. In the context of the present disclosure, “cancer antigen” or “cancer-specific antigen” refer to antigens that are common to specific hyperproliferative disorders such as cancer.

In some embodiments, the extracellular ligand-binding domain of the CAR is specific for any antigen or epitope of interest, particularly any cancer antigen or epitope of interest. As non-limiting examples, in some embodiments the antigen of the target is a tumor-associated surface antigen, such as ErbB2 (HER2/neu), carcinoembryonic antigen (CEA), epithelial cell adhesion molecule (EpCAM), epidermal growth factor receptor (EGFR), EGFR variant III (EGFRvIII), CD19, CD20, CD22, CD30, CD40, CD79b, CLL-1, disialoganglioside GD2, ductal-epithelial mucine, gp36, TAG-72, glycosphingolipids, glioma-associated antigen, B-human chorionic gonadotropin, alphafetoprotein (AFP), lectin-reactive AFP, thyroglobulin, RAGE-1, MN-CA LX, human telomerase reverse transcriptase, RU1, RU2 (AS), intestinal carboxyl esterase, mut hsp70-2, M-CSF, prostase, prostase specific antigen (PSA), PAP, NY-ESO-1, LAGA-la, p53, prostein, PSMA, surviving and telomerase, prostate-carcinoma tumor antigen-1 (PCTA-1), MAGE, ELF2M, neutrophil elastase, ephrin B2, insulin growth factor (IGF1)-1, IGF-II, IGF1 receptor, mesothelin, a major histocompatibility complex (MHC) molecule presenting a tumor-specific peptide epitope, 5T4, ROR1, Nkp30, NKG2D, tumor stromal antigens, the extra domain A (EDA) and extra domain B (EDB) of fibronectin and the A1 domain of tenascin-C (TnC A1) and fibroblast associated protein (fap); a lineage-specific or tissue specific antigen such as CD3, CD4, CD8, CD24, CD25, CD33, CD34, CD38, CD123, CD133, CD138, CTLA-4, B7-1 (CD80), B7-2 (CD86), endoglin, a major histocompatibility complex (MHC) molecule, BCMA (CD269, TNFRSF 17), IL1RAP, CS1, GPC3, FLT3, or a virus-specific surface antigen such as an HIV-specific antigen (such as HIV gp120); an EBV-specific antigen, a CMV-specific antigen, a HPV-specific antigen such as the E6 or E7 oncoproteins, a Lasse Virus-specific antigen, an Influenza Virus-specific antigen, as well as any derivate or variant of these surface markers.

In some examples, the extracellular ligand-binding domain or moiety is an antibody, or antibody fragment. An antibody fragment can, for example, be at least one portion of an antibody, that retains the ability to specifically interact with (e.g., by binding, steric hindrance, stabilizing/destabilizing, spatial distribution) an epitope of an antigen. Examples of antibody fragments include, but are not limited to, Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (Fn3) (see U.S. Pat. No. 6,703,199, which describes fibronectin polypeptide minibodies).

In some embodiments, the extracellular ligand-binding domain or moiety is in the form of a single-chain variable fragment (scFv) derived from a monoclonal antibody, which provides specificity for a particular epitope or antigen (e.g., an epitope or antigen preferentially present on the surface of a cell, such as a cancer cell or other disease-causing cell or particle). In some embodiments, the scFv is attached via a linker sequence. In some embodiments, the scFv is murine, humanized, or fully human. In certain embodiments, the scFv comprises a heavy chain variable (VH) domain and a light chain variable (VL) domain from a monoclonal antibody having specificity for a tumor cell antigen.

The extracellular ligand-binding domain of a chimeric antigen receptor can also comprise an autoantigen (see, Payne el al. (2016), Science 353 (6295): 179-184), that can be recognized by autoantigen-specific B cell receptors on B lymphocytes, thus directing T cells to specifically target and kill autoreactive B lymphocytes in antibody-mediated autoimmune diseases. Such CARs can be referred to as chimeric autoantibody receptors (CAARs), and their use is encompassed by the invention. The extracellular ligand-binding domain of a chimeric antigen receptor can also comprise a naturally-occurring ligand for an antigen of interest, or a fragment of a naturally-occurring ligand which retains the ability to bind the antigen of interest.

A CAR can comprise a transmembrane domain which links the extracellular ligand-binding domain with the intracellular signaling and co-stimulatory domains via a hinge region or spacer sequence. The transmembrane domain can be derived from any membrane-bound or transmembrane protein. For example, the transmembrane polypeptide can be a subunit of the T-cell receptor (e.g., an α, β, γ or ζ polypeptide constituting CD3 complex), IL2 receptor p55 (a chain), p75 (β chain) or γ chain, subunit chain of Fc receptors (e.g., Fcy receptor III) or CD proteins such as the CD8 alpha chain. In certain examples, the transmembrane domain is a CD8 alpha domain. Alternatively, the transmembrane domain can be synthetic and can comprise predominantly hydrophobic residues such as leucine and valine.

The hinge region refers to any oligo- or polypeptide that functions to link the transmembrane domain to the extracellular ligand-binding domain. For example, a hinge region may comprise up to 300 amino acids, preferably 10 to 100 amino acids and most preferably 25 to 50 amino acids. Hinge regions may be derived from all or part of naturally occurring molecules, such as from all or part of the extracellular region of CD8, CD4 or CD28, or from all or part of an antibody constant region. Alternatively, the hinge region may be a synthetic sequence that corresponds to a naturally occurring hinge sequence or may be an entirely synthetic hinge sequence. In particular examples, a hinge domain can comprise a part of a human CD8 alpha chain, FcyRllla receptor or IgGl. In certain examples, the hinge region can be a CD8 alpha domain.

Intracellular signaling domains of a CAR are responsible for activation of at least one of the normal effector functions of the cell in which the CAR has been placed and/or activation of proliferative and cell survival pathways. The term “effector function” refers to a specialized function of a cell. Effector function of a T cell, for example, may be cytolytic activity or helper activity including the secretion of cytokines. The intracellular stimulatory domain can include one or more cytoplasmic signaling domains that transmit an activation signal to the T cell following antigen binding. Such cytoplasmic signaling domains can include, without limitation, a CD3 zeta signaling domain.

The intracellular stimulatory domain can also include one or more intracellular co-stimulatory domains that transmit a proliferative and/or cell-survival signal after ligand binding. Such intracellular co-stimulatory domains can be any of those known in the art and can include, without limitation, those co-stimulatory domains disclosed in WO 2018/067697 including, for example, Novel 6 (“N6”). Further examples of co-stimulatory domains can include 4-1BB (CD137), CD27, CD28, CD8, OX40, CD30, CD40, PD-1, ICOS, lymphocyte function-associated antigen-1 (LFA-1), CD2, CD7, LIGHT, NKG2C, B7-H3, a ligand that specifically binds with CD83, or any combination thereof. In a particular embodiment, the co-stimulatory domain is an N6 domain. In another particular embodiment, the co-stimulatory domain is a 4-1BB co-stimulatory domain.

The CAR can be specific for any type of cancer cell. Such cancers can include, without limitation, carcinoma, lymphoma, sarcoma, blastomas, leukemia, cancers of B cell origin, breast cancer, gastric cancer, neuroblastoma, osteosarcoma, lung cancer, melanoma, prostate cancer, colon cancer, renal cell carcinoma, ovarian cancer, rhabdomyosarcoma, leukemia, and Hodgkin lymphoma. In specific embodiments, cancers and disorders include but are not limited to pre-B ALL (pediatric indication), adult ALL, mantle cell lymphoma, diffuse large B cell lymphoma, salvage post allogenic bone marrow transplantation, and the like. These cancers can be treated using a combination of CARs that target, for example, CD19, CD20, CD22, and/or ROR1. In some non-limiting examples, a genetically-modified immune cell or population thereof of the present disclosure targets carcinomas, lymphomas, sarcomas, melanomas, blastomas, leukemias, and germ cell tumors, including but not limited to cancers of B-cell origin, neuroblastoma, osteosarcoma, prostate cancer, renal cell carcinoma, liver cancer, gastric cancer, bone cancer, pancreatic cancer, skin cancer, cancer of the head or neck, breast cancer, lung cancer, cutaneous or intraocular malignant melanoma, renal cancer, uterine cancer, ovarian cancer, colorectal cancer, colon cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, environmentally induced cancers including those induced by asbestos, multiple myeloma, Hodgkin lymphoma, non-Hodgkin lymphomas, acute myeloid lymphoma, chronic myelogenous leukemia, chronic lymphoid leukemia, immunoblastic large cell lymphoma, acute lymphoblastic leukemia, mycosis fungoides, anaplastic large cell lymphoma, and T-cell lymphoma, and any combinations of said cancers. In certain embodiments, cancers of B-cell origin include, without limitation, B-lineage acute lymphoblastic leukemia, B-cell chronic lymphocytic leukemia, B-cell lymphoma, diffuse large B cell lymphoma, pre-B ALL (pediatric indication), mantle cell lymphoma, follicular lymphoma, marginal zone lymphoma, Burkitt's lymphoma, and multiple myeloma. In some examples, cancers can include, without limitation, cancers of B cell origin or multiple myeloma. In some examples, the cancer of B cell origin is acute lymphoblastic leukemia (ALL), chronic lymphocytic leukemia (CLL), small lymphocytic lymphoma (SLL), or non-Hodgkin lymphoma (NHL). In some examples, the cancer of B cell origin is mantle cell lymphoma (MCL) or diffuse large B cell lymphoma (DLBCL).

In other embodiments, the donor template introduced into the genome of the cell comprises a nucleic acid sequence encoding an exogenous T cell receptor (TCR). Such exogenous T cell receptors can comprise alpha and beta chains or, alternatively, may comprise gamma and delta chains. Exogenous TCRs useful in the invention may have specificity to any antigen or epitope of interest. For example, exogenous TCRs can have specificity for any cancer antigen or any type of cancer cell described herein.

Cells transfected with the lipid nanoparticles described herein can be further modified to express one or more inducible suicide genes, the induction of which provokes cell death and allows for selective destruction of the cells in vitro or in vivo. In some examples, a suicide gene can encode a cytotoxic polypeptide, a polypeptide that has the ability to convert a non-toxic pro-drug into a cytotoxic drug, and/or a polypeptide that activates a cytotoxic gene pathway within the cell. That is, a suicide gene is a nucleic acid that encodes a product that causes cell death by itself or in the presence of other compounds. A representative example of such a suicide gene is one that encodes thymidine kinase of herpes simplex virus. Additional examples are genes that encode thymidine kinase of varicella zoster virus and the bacterial gene cytosine deaminase that can convert 5-fluorocytosine to the highly toxic compound 5-fluorouracil. Suicide genes also include as non-limiting examples genes that encode caspase-9, caspase-8, or cytosine deaminase. In some examples, caspase-9 can be activated using a specific chemical inducer of dimerization (CID). A suicide gene can also encode a polypeptide that is expressed at the surface of the cell that makes the cells sensitive to therapeutic and/or cytotoxic monoclonal antibodies. In further examples, a suicide gene can encode recombinant antigenic polypeptide comprising an antigenic motif recognized by the anti-CD20 mAb Rituximab and an epitope that allows for selection of cells expressing the suicide gene. See, for example, the RQR8 polypeptide described in WO2013153391, which comprises two Rituximab-binding epitopes and a QBEnd10-binding epitope. For such a gene, Rituximab can be administered to a subject to induce cell depletion when needed. In further examples, a suicide gene may include a QBEnd10-binding epitope expressed in combination with a truncated EGFR polypeptide.

### 2.7 Populations of Eukaryotic Cells

The present invention further includes populations of eukaryotic cells (e.g., T cell or NK cells) prepared by any of the methods described herein. In certain embodiments, the populations of eukaryotic cells prepared by the methods disclosed herein are electroporation naïve.

In some embodiments of the invention, the eukaryotic cells (e.g., T cells or NK cells) are genetically-modified to knock-out expression of an endogenous protein by inactivating a target gene. In some such embodiments, the invention provides populations of eukaryotic cells wherein about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, or up to 100%, of cells in the population are genetically-modified to knock-out expression of an endogenous protein by inactivating a target gene. In further such embodiments, between about 5% and about 75%, between about 10% and about 75%, between about 20% and about 75%, between about 30% and about 75%, between about 40% and about 75%, between about 45% and about 75%, between about 50% and about 75%, between about 55% and about 75%, between about 60% and about 75%, between about 65% and about 75%, between about 70% to about 75%, between about 75% and about 95%, between about 80% and about 95%, between about 85% and about 95% between about 90% and about 95%, between about 90% and about 100%, or between about 95% and about 100% of the cells in the population are such genetically-modified cells.

In some embodiments of the invention, the eukaryotic cells (e.g., T cells or NK cells) are genetically-modified to knock-in a transgene into the genome of the cells. In some such embodiments, the invention provides populations of eukaryotic cells wherein about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, or up to 100%, of cells in the population are genetically-modified to express a transgene that has been knocked-in to the genome of the cells. In further such embodiments, between about 5% and about 75%, between about 10% and about 75%, between about 20% and about 75%, between about 30% and about 75%, between about 40% and about 75%, between about 45% and about 75%, between about 50% and about 75%, between about 55% and about 75%, between about 60% and about 75%, between about 65% and about 75%, between about 70% to about 75%, between about 75% and about 95%, between about 80% and about 95%, between about 85% and about 95%, between about 90% and about 95%, between about 90% and about 100%, or between about 95% and about 100% of the cells in the population are such genetically-modified cells.

In other particular embodiments, the eukaryotic cells (e.g., T cells or NK cells) exhibit a partial reduction in expression of an endogenous protein, for example by expression of an RNAi molecule. In some such embodiments, the invention provides populations of eukaryotic cells wherein about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, or up to 100%, of cells in the population are eukaryotic cells exhibiting a partial reduction in the expression of an endogenous protein. In further such embodiments, between about 5% and about 75%, between about 10% and about 75%, between about 20% and about 75%, between about 30% and about 75%, between about 40% and about 75%, between about 45% and about 75%, between about 50% and about 75%, between about 55% and about 75%, between about 60% and about 75%, between about 65% and about 75%, or between about 70% to about 75% of the cells in the population are such eukaryotic cells. The reduction of expression of the endogenous protein can be by any amount between about 1% to about 99% of wild-type levels in a control cell.

In some particular embodiments of the invention, the eukaryotic cells are genetically-modified human T cells or NK cells that express a CAR or an exogenous TCR, and further comprise an inactivated TCR alpha gene, TRAC gene, TCR beta gene, and/or TRBC gene. In some such embodiments, the invention provides populations of human T cells or NK cells wherein about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, or up to 100%, of cells in the population are genetically-modified human T or NK cells expressing a CAR or an exogenous TCR, and comprising an inactivated TCR alpha gene, TRAC gene, TCR beta gene, and/or TRBC gene. In further such embodiments, between about 5% and about 75%, between about 10% and about 75%, between about 20% and about 75%, between about 30% and about 75%, between about 40% and about 75%, between about 45% and about 75%, between about 50% and about 75%, between about 55% and about 75%, between about 60% and about 75%, between about 65% and about 75%, or between about 70% to about 75% of the cells in the population are such genetically-modified human T cells or NK cells. Such cells do not have detectable cell-surface expression of an endogenous alpha/beta TCR due to the disruption of a gene encoding a component of the endogenous alpha/beta TCR complex.

### 2.8 Pharmaceutical Compositions

The invention also provides pharmaceutical compositions comprising a pharmaceutically-acceptable carrier and a eukaryotic cell described herein (e.g., a T cell or NK cell), or a population of eukaryotic cells, wherein the cells or populations are prepared according to the method disclosed herein. Such pharmaceutical compositions can be prepared in accordance with known techniques. See, e.g., Remington, The Science and Practice of Pharmacy (21st ed. 2005). In the manufacture of a pharmaceutical formulation according to the invention, cells are typically admixed with a pharmaceutically acceptable carrier and the resulting composition is administered to a subject. The carrier must, of course, be acceptable in the sense of being compatible with any other ingredients in the formulation and must not be deleterious to the subject. In some embodiments, pharmaceutical compositions of the invention can further comprise one or more additional agents useful in the treatment of a disease in the subject. In additional embodiments, pharmaceutical compositions of the invention can further include biological molecules, such as cytokines (e.g., IL-2, IL-7, IL-15, and/or IL-21), which may promote in viva cell proliferation and engraftment of cells (e.g., T cells). Pharmaceutical compositions comprising eukaryotic cells of the invention can be administered in the same composition as an additional agent or biological molecule or, alternatively, can be co-administered in separate compositions.

The present disclosure also provides eukaryotic cells (e.g., T cell or NK cells), or populations thereof, described herein for use as a medicament. The present disclosure further provides the use of eukaryotic cells, or populations thereof, described herein in the manufacture of a medicament for treating a disease in a subject in need thereof. In one such aspect, the medicament is useful for cancer immunotherapy in subjects in need thereof, wherein the eukaryotic cells are genetically modified human immune cells (e.g., T cells or NK cells) expressing a CAR or an exogenous TCR.

Pharmaceutical compositions of the invention can be useful for treating any disease state that can be targeted by adoptive immunotherapy. In a particular embodiment, the pharmaceutical compositions and medicaments of the invention are useful in the treatment of cancer including, for example, types of cancer described elsewhere herein.

In some of these embodiments wherein cancer is treated with the presently disclosed eukaryotic cells, or populations thereof, the subject administered the eukaryotic cells, or populations thereof, is further administered an additional therapeutic, such as radiation, surgery, or a chemotherapeutic agent.

### 2.9 Kits

Another aspect of the invention is a kit for transfecting a eukaryotic cell with a nucleic acid. In some embodiments, the kit includes a lipid nanoparticle composition described herein, which is bound to an apolipoprotein (e.g., ApoE). In some embodiments, the lipid nanoparticle composition is provided in a vial. In some embodiments, the kit further comprises a reagent that enhances the transfection efficiency of the lipid nanoparticle composition. In some further embodiments, the kit includes packaging and instructions for use thereof.

### 2.10 Methods of Administering Eukaryotic Cells

The invention also provides methods of treatment comprising administering an effective amount of eukaryotic cells (e.g., T cells or NK cells), or populations thereof, of the present disclosure to a subject in need thereof. In particular embodiments, the pharmaceutical compositions described herein are administered to a subject in need thereof. For example, an effective amount of a population of eukaryotic cells can be administered to a subject having a disease. In particular embodiments, the disease can be cancer, and administration of the eukaryotic cells of the invention represent an immunotherapy. The administered cells are able to reduce the proliferation, reduce the number, or kill target cells in the recipient. Unlike antibody therapies, eukaryotic cells of the present disclosure are able to replicate and expand in vivo, resulting in long-term persistence that can lead to sustained control of a disease.

In particular embodiments of the presently disclosed methods, the subject can be a mammal, such as a human.

Examples of possible routes of administration include parenteral, (e.g., intravenous (IV), intramuscular (IM), intradermal, subcutaneous (SC), or infusion) administration. Moreover, the administration may be by continuous infusion or by single or multiple boluses. In specific embodiments, the agent is infused over a period of less than about 12 hours, 6 hours, 4 hours, 3 hours, 2 hours, or 1 hour. In still other embodiments, the infusion occurs slowly at first and then is increased over time.

In some embodiments, a eukaryotic cell (e.g., a T cell or NK cell), or population thereof, of the present disclosure targets a tumor (i.e., cancer) antigen for the purposes of treating cancer including, for example, types of cancer described elsewhere herein.

When an “effective amount” or “therapeutic amount” is indicated, the precise amount to be administered can be determined by a physician with consideration of individual differences in age, weight, tumor size (if present), extent of infection or metastasis, and condition of the patient (subject). In some embodiments, a pharmaceutical composition comprising the eukaryotic cells (e.g., T cells or NK cells), or populations thereof, described herein is administered at a dosage of 10⁴ to 10⁹ cells/kg body weight, including all integer values within those ranges. In further embodiments, the dosage is 10⁵ to 10⁷ cells/kg body weight, including all integer values within those ranges. In further embodiments, the dosage is 10⁵ to 10⁶ cells/kg body weight, including all integer values within those ranges. In some embodiments, cell compositions are administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

In some embodiments, administration of eukaryotic cells (e.g., T cells or NK cells), or populations thereof, described herein reduce at least one symptom of a target disease or condition, such as a cancer. Symptoms of cancers are well known in the art and can be determined by known techniques.

### EXAMPLES

This invention is further illustrated by the following examples, which should not be construed as limiting. Those skilled in the art will recognize, or be able to ascertain, using no more than routine experimentation, numerous equivalents to the specific substances and procedures described herein. Such equivalents are intended to be encompassed in the scope of the claims that follow the examples below.

### Example 1

### Use of ApoE-Conjugated LNPs in Presence of High Serum Conditions to Deliver Nuclease mRNA for TCR Gene Knock Out in Primary Human T Cells

### Methods

The purpose of this experiment was to evaluate LNP and LNP ApoE conjugate formulations for delivering mRNA in the presence of serum in the production of CAR T cells.

### Lipid Nanoparticle Formulations

The lipid materials used for the formulation of LNPs included DLin-MC3-DMA, cholesterol, a DSPC, and total PEG lipids dissolved at a molar ratio of 40:48.5:10:1.5 in ethanol at a total lipid concentration of 15 mM (Formulated at constant N:P=8). The PEG lipids included DMG-PEG2000 and a functionalized DSPE-PEG2000, chemically-modified to include an amine, carboxylic acid, or maleimide group, at a 149:1 ratio DMG-PEG to DSPE-PEG.

The mix of lipids was stored at −80 C and thawed by heating to 50 C in heat block. Lipid mix was taken off heat and vortexed immediately before use in formulation. mRNA material coding for an engineered meganuclease referred to as TRC 1-2L.1592 (SEQ ID NO: 4), which binds and cleaves a recognition sequence (SEQ ID NO: 2) in the human TRAC gene, included a clean cap 1 structure with unmodified uridine. mRNA was stored at −80 C and thawed at room temperature. Once thawed, the mRNA was diluted to 0.1 mg/mL in a 50 mM citrate buffer at pH=4.0. Microfluidic mixing of the mRNA and lipid solutions at a 3:1 ratio into an exchange buffer (PBS pH=7.4) via Precision Nanosystems Benchtop Nanoassembler was performed. Final solution in the exchange buffer was collected, concentrated, and analyzed for physical characteristics such as size, PDI, and zeta potential, as well as for encapsulation efficiency. All LNPs were centrifuged to concentrate to final concentration at 0.1 mg/ml mRNA.

To initiate conjugation reactions of LNPs containing DSPE-PEG2000-NH2 group, ApoE3 solution was added followed by addition of EDCI. To initiate conjugation reactions of LNPs containing DSPE-PEG2000-COOH group, EDCI solution was added followed by additional of ApoE. To initiate conjugation reactions of LNPs containing DSPE-PEG2000-Maleimide group, ApoE solution was added. These solutions were kept on gentle stirring for 8 hrs and checked intermittently to ensure no aggregation in solution. After mixing, LNPs were dialysed again in 1×PBS overnight. Final solution was collected and analyzed for physical characteristics such as size, PDI, and zeta potential, as well as for encapsulation efficiency. Following buffer exchange, formulation was diluted to desired concentration in PBS before addition to cell culture media. The formulation was added to human donor T-cells in either a media containing no serum and 1 ug/mL ApoE, or a high human serum condition (%20) with no supplementation of ApoE to assess efficacy of LNP formulations to deliver mRNA encoding a TRC nuclease to edit at the TCR locus and reduce endogenous TCR on the cell surface, measured by CD3 staining and flow cytometry analysis.

### T Cell Culture and Transfection

In this study, an apheresis sample was drawn from a healthy, informed, and compensated donor, and the T cells were enriched using the CD3 positive selection kit II in accord with the manufacturer's instructions (Stein Cell Technologies). T cells were activated using MACS GMP T Cell TransAct (Miltenyi Biotec) in Xuri T cell expansion medium (GE) supplemented with 5% human serum AB (Gemini) and 10 ng/ml IL-2 (CellGenix). After 3 days of stimulation, cells were collected, washed and resuspended in serum supplemented medium (20%) or in serum void media with 1 ug/mL ApoE. Samples of 5 e5 cells/mL were treated with 2.5 ug/mL of LNP formulation with mRNA coding for TRC nuclease.

### Analysis

Flow cytometry was used to assess cell phenotype of cells at clay 7. Cells were collected and stained with Ghost Dye Violet 510 (Tonbo Biosciences), anti-human CD3-BV421, clone OKT3 (BioLegend) or anti-human CD3-BV711, clone UCHT1 (BioLegend), anti-human CD4-FITC, clone OKT4 (BioLegend) or anti-human CD4-APC, clone OKT4 (BioLegend), anti-human CD8-PE, clone RPA-T8 (BioLegend) or anti-human CD8-BV711, clone RPA-T8 (BioLegend), anti-human CD45RO clone UCHL1 (BioLegend), anti-human CD62L, clone DREG-56 (BD Biosciences) and anti-FMC63 recombinant antibody-AF647, clone VM16 (BioLegend). Cells were then washed, resuspended in PBS (Gibco) and analyzed on CytoFLEX LX (Beckman Coulter). Post-run analysis was completed using CytExpert software (Beckman Coulter). Cells were gated on FSC vs SSC scatter followed by singlets then live.

### Results and Conclusions

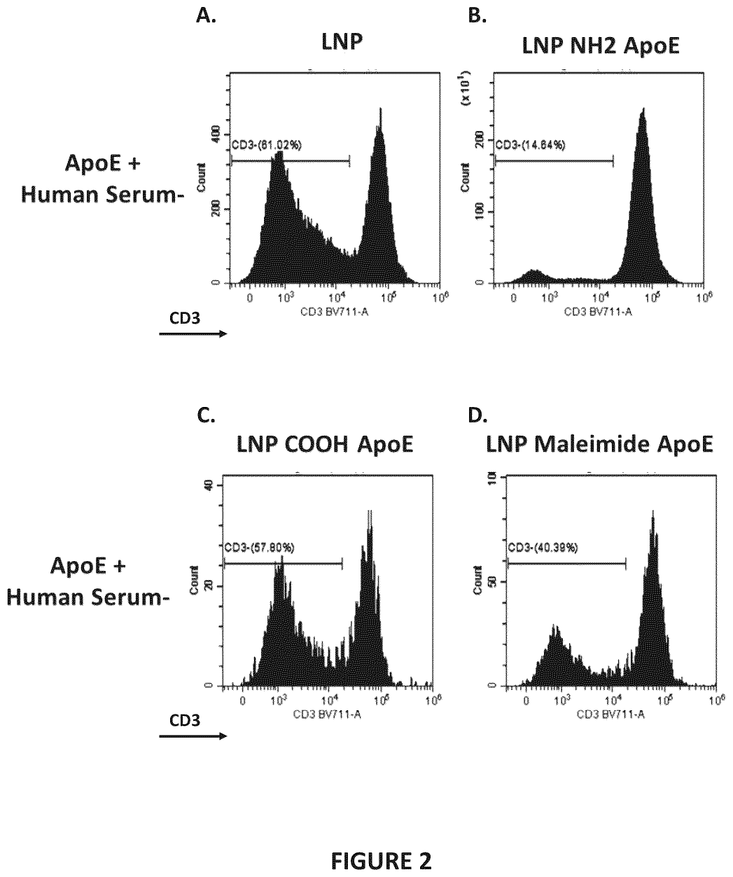

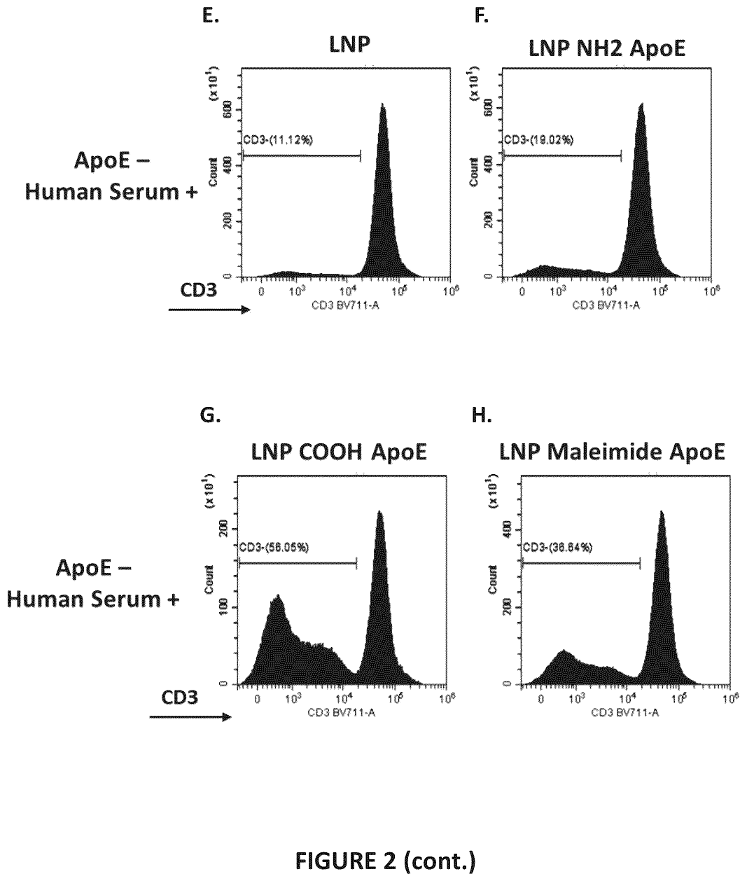

Flow cytometry results summarizing the CD3+/− populations after ApoE-conjugated LNP treatment for a CAR T production process, in either a high serum void of ApoE supplementation, or in a no serum condition with ApoE supplementation, are illustrated in FIG. 2.

This study evaluated the impact of ApoE-conjugated LNPs to generate high knock-out (KO) % by overcoming quenching factors in high serum conditions with no supplementation of ApoE in primary T cell culture media. This test illustrates the LNP formulations' ability to transfect T cells with mRNA encoding engineered nucleases and knockout the TCR locus despite high serum conditions, without the need for ApoE supplementation, when the ApoE is conjugated to the LNP during the formulation process. Using CD3 KO analysis, it was observed that the formulation was able to maintain transfection and KO potency through 20% serum supplemented media, while LNPs without ApoE-conjugation resulted in reduced potency in high serum conditions. Therefore, this data demonstrates that ApoE/LNP formulation complexes can transfect in the presence of high serum conditions to maintain potency.

### Example 2

### Use of ApoE-Conjugated LNPs in Presence of High Serum Conditions to Deliver Nuclease mRNA for Production of CAR T Cells

### Methods

The purpose of this experiment was to evaluate LNP and LNP ApoE conjugate formulations for delivering mRNA in the presence of serum in the production of CAR T cells.

### Lipid Nanoparticle Formulation

The lipid materials used for the formulation of LNP include DLin-MC3-DMA, cholesterol, DSPC, and total PEG lipids dissolved at a molar ratio of 40:48.5:10:1.5 in ethanol at a total lipid concentration of 15 mM (Formulated at constant N:P=8). The PEG lipids included DMG-PEG2000 and a functionalized DSPE-PEG2000, chemically-modified to include an amine, carboxylic acid, or maleimide group, at a 149:1 ratio DMG-PEG to DSPE-PEG.

The mix of lipids was stored at −80 C and thawed by heating to 50 C in heat block. Lipid mix was taken off heat and vortexed immediately before use in formulation. mRNA material coding for an engineered meganuclease referred to as TRC 1-2L.1592 (SEQ ID NO: 4), which binds and cleaves a recognition sequence (SEQ ID NO: 2) in the human TRAC gene, included a clean cap 1 structure with unmodified uridine. mRNA was stored at −80 C and thawed at room temperature. mRNA was stored at −80 C and thawed at room temperature. Once thawed, the mRNA was diluted to 0.1 mg/mL in a 50 mM citrate buffer at pH=4.0. Microfluidic mixing of the mRNA and lipid solutions at a 3:1 ratio into an exchange buffer (PBS pH=7.4) via Precision Nanosystems Benchtop Nanoassembler was performed. Final solution in the exchange buffer was collected, concentrated, and analyzed for physical characteristics such as size, PDI, and zeta potential, as well as for encapsulation efficiency. All LNPs were centrifuged to concentrate to final concentration at 0.1 mg/ml mRNA. To initiate conjugation reactions of LNPs containing DSPE-PEG2000-NH2 group, ApoE3 solution was added followed by additional of EDCI. To initiate conjugation reactions of LNPs containing DSPE-PEG2000-COOH group, EDCI solution was added followed by additional of ApoE. To initiate conjugation reactions of LNPs containing DSPE-PEG2000-Maleimide group, ApoE solution was added. These solutions were kept on gentle stirring for 8 hrs and checked intermittently to ensure no aggregation in solution. After mixing, LNPs were dialysed again in 1×PBS overnight. Final solution was collected and analyzed for physical characteristics such as size, PDI, and zeta potential, as well as for encapsulation efficiency. Following buffer exchange, formulation was diluted to desired concentration in PBS before addition to cell culture media. The formulation was added to human donor T cells in either a media containing no serum and 1 ug/mL ApoE, or a high human serum condition (%20) with no supplementation of ApoE to assess efficacy of LNP formulations to deliver mRNA encoding a TRC nuclease to edit at the TCR locus and reduce TCR on the cell surface, measured by CD3 staining and flow cytometry analysis. Further addition of 125K MOI of AAV (serotype 6) carrying a CD19-specific CAR transgene is added following LNP addition to assess gene knock in and CAR T production via flow cytometry analysis.

### T Cell Culture and Transfection

In this study, an apheresis sample was drawn from a healthy, informed, and compensated donor, and the T cells were enriched using the CD3 positive selection kit II in accord with the manufacturer's instructions (Stem Cell Technologies). T cells were activated using MACS GMP T Cell TransAct (Miltenyi Biotec) in Xuri T cell expansion medium (GE) supplemented with 5% human serum AB (Gemini) and 10 ng/mi (CeliGenix). After 3 days of stimulation, cells were collected, washed and resuspended in serum supplemented medium (20%) or in serum void media with 1 ug/mL ApoE. Samples of 5 e5 cells/mL were treated with 2.5 ug/mL of LNP formulation with mRNA coding for TRC nuclease along with 125K MOI of AAV carrying the CAR transgene.

### Analysis

Flow cytometry was used to assess cell phenotype of cells at day 7. Cells were collected and stained with Ghost Dye Violet 510 (Tonbo Biosciences), anti-human CD3-BV421, clone OKT3 (BioLegend) or anti-human CD3-BV711, clone UCHT1 (BioLegend), anti-human CD4-FITC, clone OKT4 (BioLegend) or anti-human CD4-APC, clone OKT4 (BioLegend), anti-human CD8-PE, clone RPA-T8 (BioLegend) or anti-human CD8-BV711, clone RPA-T8 (BioLegend), anti-human CD45RO, clone UCHL1 (BioLegend), anti-human CD62L, clone DREG-56 (BD Biosciences) and anti-FMC63 recombinant antibody-AF647, clone V1\416 (BioLegend). Cells were then washed, resuspended in PBS (Gibco) and analyzed on CytoFLEX LX (Beckman Coulter). Post-run analysis was completed using CytExpert software (Beckman Coulter). Cells were gated on FSC vs SSC scatter followed by singlets then live.

### Results and Conclusions

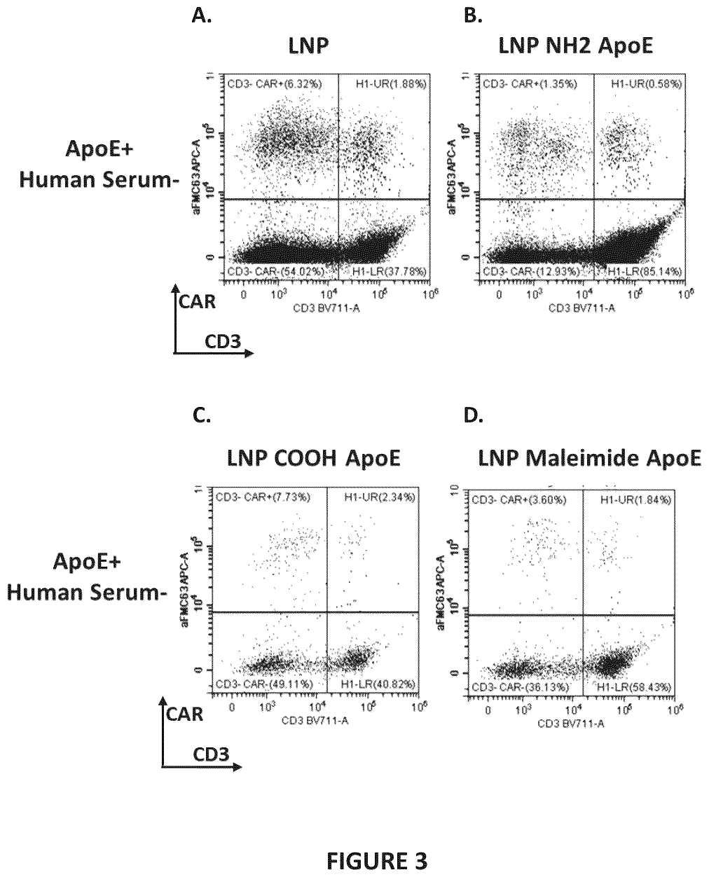

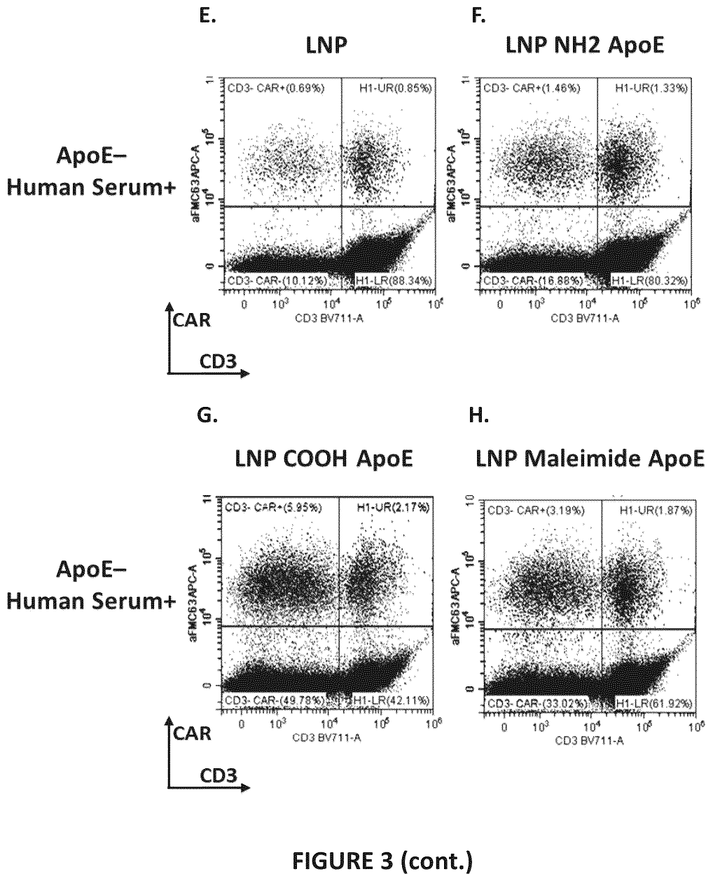

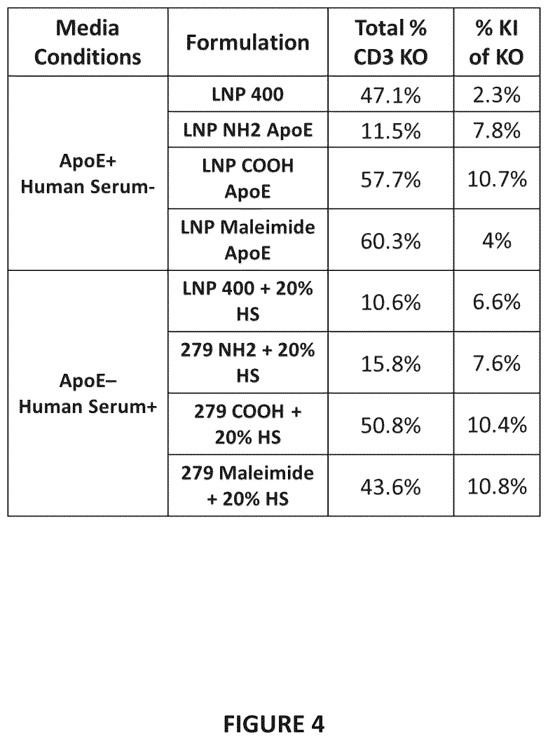

Flow cytometry results, and a table summarizing the CD3+/−KO % and KI % populations after ApoE-conjugated LNP treatment in a CAR T production process in either a high serum condition void of ApoE supplementation, or in a no serum condition with ApoE supplementation, is are shown in FIGS. 3 and 4, respectively.

This study evaluated the impact of ApoE-conjugated LNPs to generate high KO % and KI % by overcoming quenching factors in high serum conditions with no supplementation of ApoE in primary T cell culture media. This test illustrated the LNP formulations' ability to transfect T cells with mRNA encoding a engineered nuclease and knockout the TCR locus, while also allowing for knock-in of a transgene encoding a CAR in high serum conditions without the need for ApoE supplementation when the ApoE is directly conjugated to the LNP during the formulation process. Through CD3 KO and CAR KI analysis, it was observed that the formulation was able to maintain potency of gene knockout and transgene knock-in despite the use of 20% serum supplemented media, while LNPs without ApoE-conjugation resulted in reduced potency in high serum conditions. Therefore, this data demonstrates that ApoE/LNP formulation complexes can transfect in the presence of high serum conditions to maintain potency and produce CAR T cells.

What is claimed is:

1. A lipid nanoparticle composition comprising lipid nanoparticles comprising:
(a) one or more cationic lipids;
(b) one or more non-cationic lipids;
(c) one or more lipid conjugates; and
(d) an apolipoprotein covalently bound to at least one of said one or more cationic lipids, at least one of said one or more non-cationic lipids, or at least one of said one or more lipid conjugates.

2. The lipid nanoparticle composition of claim 1, wherein:
(a) at least one of said one or more cationic lipids bound to said apolipoprotein is a chemically modified cationic lipid;
(b) at least one of said one or more non-cationic lipids bound to said apolipoprotein is a chemically modified non-cationic lipid; or
(c) at least one of said one or more lipid conjugates bound to said apolipoprotein is a chemically modified lipid conjugate.

3. The lipid nanoparticle composition of claim 2, wherein said chemically modified cationic lipid, said chemically modified non-cationic lipid, or said chemically modified lipid conjugate, is chemically modified at its terminus, and wherein said apolipoprotein is bound at said terminus.

4. The lipid nanoparticle composition of claim 2 or 3, wherein the modification of said chemically modified cationic lipid, said chemically modified non-cationic lipid, or said chemically modified lipid conjugate, comprises an introduced amino group, a carboxyl group, a hydroxyl group, a sulfhydryl group, a maleimide group, an acyl halide group, an acetyl halide group, an aldehyde group, an azo group, an azide group, an alkyne group, an alkene group, a haloalkane group, a phosphine group, an imine group, a thiol group, a sulfoxide group, a sulfone group, a sulfonic acid group, a sulfide group, a peroxide group, a chelating group, an ester group, an epoxide group, a nitrone group, a cyclooctynes group, a sulfonyl halides group, a β-propiolactone group, a γ-butyrolactone group, a β-lactam group, a boronic acid group, or an aryl urea with one nitrogen being in aliphatic ring group.

5. The lipid nanoparticle composition of any one of claims 1-4, wherein said apolipoprotein is covalently bound by an amide bond, a thioester bond, a disulfide bond, a hydrazine bond, an imine bond, an azole bond, or triazole bond.

6. The lipid nanoparticle composition of any one of claims 2-5, wherein:
(a) said one or more cationic lipids includes at least one chemically modified cationic lipid and at least one unmodified cationic lipid;
(b) said one or more non-cationic lipids includes at least one chemically modified non-cationic lipid and at least one unmodified non-cationic lipid; or
(c) said one or more lipid conjugates includes at least one chemically modified lipid conjugate and at least one unmodified lipid conjugate.

7. The lipid nanoparticle composition of claim 6, wherein:
(a) said chemically modified cationic lipid is derived from said unmodified cationic lipid;
(b) said chemically modified non-cationic lipid is derived from said unmodified non-cationic lipid; or
(c) said chemically modified lipid conjugate is derived from said unmodified lipid conjugate.

8. The lipid nanoparticle composition of claim 6, wherein:
(a) said chemically modified cationic lipid is not derived from said unmodified cationic lipid;
(b) said chemically modified non-cationic lipid is not derived from said unmodified non-cationic lipid; or
(c) said chemically modified lipid conjugate is not derived from said unmodified lipid conjugate.

9. The lipid nanoparticle composition of any one of claims 1-8, wherein said apolipoprotein is an apolipoprotein A (ApoA), apolipoprotein B (ApoB), apolipoprotein C (ApoC), apolipoprotein D (ApoD), apolipoprotein E (ApoE), apolipoprotein H (ApoH), apolipoprotein L (ApoL), apolipoprotein M (ApoM), or apolipoprotein (a) (Apo(a)).

10. The lipid nanoparticle composition of any one of claims 1-9, wherein said apolipoprotein is ApoE.

11. The lipid nanoparticle composition of any one of claims 1-10, wherein said apolipoprotein is ApoE isoform 2, ApoE isoform 3, or ApoE isoform 4.

12. The lipid nanoparticle composition of any one of claims 1-11, wherein the total molar concentration of said one or more cationic lipids is from about 20% to about 80%, from about 30% to about 70%, from about 40% to about 60%, or from about 45% to about 55% of the total lipid molar concentration.

13. The lipid nanoparticle composition of any one of claims 1-12, wherein the total molar concentration of the one or more cationic lipids is about 20%, about 20.5%, about 21%, about 21.5%, about 22%, about 22.5%, about 23%, about 23.5%, about 24%, about 24.5%, about 25%, about 25.5%, about 26%, about 26.5%, about 27%, about 27.5%, about 28%, about 28.5%, about 29%, about 29.5%, about 30%, about 30.5%, about 31%, about 31.5%, about 32%, about 32.5%, about 33%, about 33.5%, about 34%, about 34.5%, about 35%, about 35.5%, about 36%, about 36.5%, about 37%, about 37.5%, about 38%, about 38.5%, about 39%, about 39.5%, about 40%, about 40.5%, about 41%, about 41.5%, about 42%, about 42.5%, about 43%, about 43.5%, about 44%, about 44.5%, about 45%, about 45.5%, about 46%, about 46.5%, about 47%, about 47.5%, about 48%, about 48.5%, about 49%, about 49.5%, about 50%, about 50.5%, about 51%, about 51.5%, about 52%, about 52.5%, about 53%, about 53.5%, about 54%, about 54.5%, about 55%, about 55.5%, about 56%, about 56.5%, about 57%, about 57.5%, about 58%, about 58.5%, about 59%, about 59.5%, about 60%, about 60.5%, about 61%, about 61.5%, about 62%, about 62.5%, about 63%, about 63.5%, about 64%, about 64.5%, about 65%, about 65.5%, about 66%, about 66.5%, about 67%, about 67.5%, about 68%, about 68.5%, about 69%, about 69.5%, about 70%, about 70.5%, about 71%, about 71.5%, about 72%, about 72.5%, about 73%, about 73.5%, about 74%, about 74.5%, about 75%, about 75.5%, about 76%, about 76.5%, about 77%, or about 77.5% of the total lipid molar concentration.

14. The lipid nanoparticle composition of any one of claims 1-13, wherein the total molar concentration of said one or more non-cationic lipids is from about 20% to about 100%, from about 20% to about 90%, from about 20% to about 80%, from about 25% to about 100%, from about 25% to about 90%, from about 25% to about 89.9%, from about 30% to about 70%, from about 40% to about 70%, from about 40% to about 60%, or from about 45% to about 55% of the total lipid molar concentration.

15. The lipid nanoparticle composition of any one of claims 1-14, wherein the total molar concentration of said one or more non-cationic lipids is about 25%, about 25.4%, about 25.9%, about 26%, about 26.4%, about 26.5%, about 26.9%, about 27%, about 27.4%, about 27.5%, about 27.9%, about 28%, about 28.4%, about 28.5%, about 28.9%, about 29%, about 29.4%, about 29.5%, about 29.9%, about 30%, about 30.4%, about 30.5%, about 30.9%, about 31%, about 31.4%, about 31.5%, about 31.9%, about 32%, about 32.4%, about 32.5%, about 32.9%, about 33%, about 33.4%, about 33.5%, about 33.9%, about 34%, about 34.4%, about 34.5%, about 34.9%, about 35%, about 35.4%, about 35.5%, about 35.9%, about 36%, about 36.4%, about 36.5%, about 36.9%, about 37%, about 37.4%, about 37.5%, about 37.9%, about 38%, about 38.4%, about 38.5%, about 38.9%, about 39%, about 39.4%, about 39.5%, about 39.9%, about 40%, about 40.4%, about 40.5%, about 40.9%, about 41%, about 41.4%, about 41.5%, about 41.9%, about 42%, about 42.4%, about 42.5%, about 42.9%, about 43%, about 43.4%, about 43.5%, about 43.9%, about 44%, about 44.4%, about 44.5%, about 44.9%, about 45%, about 45.4%, about 45.5%, about 45.9%, about 46%, about 46.4%, about 46.5%, about 46.9%, about 47%, about 47.4%, about 47.5%, about 47.9%, about 48%, about 48.4%, about 48.5%, about 48.9%, about 50%, about 50.4%, about 50.5%, about 50.9%, about 51%, about 51.4%, about 51.5%, about 51.9%, about 52%, about 52.4%, about 52.5%, about 52.9%, about 53%, about 53.4%, about 53.5%, about 53.9%, about 54%, about 54.4%, about 54.5%, about 54.9%, about 55%, about 55.4%, about 55.5%, about 55.9%, about 56%, about 56.4%, about 56.5%, about 56.9%, about 57%, about 57.4%, about 57.5%, about 57.9%, about 58%, about 58.4%, about 58.5%, about 58.9%, about 59%, about 59.4%, about 59.5%, about 59.9%, about 60%, about 60.4%, about 60.5%, about 60.9%, about 61%, about 61.4%, about 61.5%, about 61.9%, about 62%, about 62.4%, about 62.5%, about 62.9%, about 63%, about 63.4%, about 63.5%, about 63.9%, about 64%, about 64.4%, about 64.5%, about 64.9%, about 65%, about 65.4%, about 65.5%, about 65.9%, about 66%, about 66.4%, about 66.5%, about 66.9%, about 67%, about 67.4%, about 67.5%, about 67.9%, about 68%, about 68.4%, about 68.5%, about 68.9%, about 69%, about 69.4%, about 69.5%, about 69.9%, about 70%, about 70.4%, about 70.5%, about 70.9%, about 71%, about 71.4%, about 71.5%, about 71.9%, about 72%, about 72.4%, about 72.5%, about 72.9%, about 73%, about 73.4%, about 73.5%, about 73.9%, about 74%, about 74.4%, about 74.5%, about 74.9%, about 75%, about 75.4%, about 75.5%, about 75.9%, about 76%, about 76.4%, about 76.5%, about 76.9%, about 77%, about 77.4%, about 77.5%, about 77.9%, about 78%, about 78.4%, about 78.5%, about 78.9%, about 79%, about 79.4%, about 79.5%, about 79.9%, about 80%, about 80.4%, about 80.5%, about 80.9%, about 81%, about 81.4%, about 81.5%, about 81.9%, about 82%, about 82.4%, about 82.5%, about 82.9%, about 83%, about 83.4%, about 83.5%, about 83.9%, about 84%, about 84.4%, about 84.5%, about 84.9%, about 85%, about 85.4%, about 85.5%, about 85.9%, about 86%, about 86.4%, about 86.5%, about 86.9%, about 87%, about 87.4%, about 87.5%, about 87.9%, about 88%, about 88.4%, about 88.5%, about 88.9%, about 89%, about 89.4%, about 89.5%, or about 89.9% of the total lipid molar concentration.

16. The lipid nanoparticle composition of any one of claims 1-15, wherein said one or more non-cationic lipids includes one or more phospholipids.

17. The lipid nanoparticle composition of claim 16, wherein the total molar concentration of said one or more phospholipids is from about 0% to about 30%, from about 2.5% to about 25%, from about 5% to about 20%, from about 5% to about 15%, or from about 7.5% to about 12.5%, or about 10% of the total lipid molar concentration.

18. The lipid nanoparticle composition of claim 16 or 17, wherein the total molar concentration of said one or more phospholipids is about 5%, about 7.5%, about 10%, about 12.5%, about 15%, about 17.5%, or about 20% of the total lipid molar concentration.

19. The lipid nanoparticle composition of any one of claims 1-18, wherein said one or more non-cationic lipids includes one or more steroids.

20. The lipid nanoparticle composition of claim 19, wherein the total molar concentration of said one or more steroids is from about 20% to about 70%, from about 20% to about 69.9%, from about 20% to about 60%, from about 25% to about 55%, from about 30% to about 50%, or from about 35% to about 40% of the total lipid molar concentration.

21. The lipid nanoparticle composition of claim 19 or 20, wherein the total molar concentration of said one or more steroids is about 20%, about 20.4%, about 20.5%, about 20.9%, about 21%, about 21.4%, about 21.5%, about 21.9%, about 22%, about 22.4%, about 22.5%, about 22.9%, about 23%, about 23.4%, about 23.5%, about 23.9%, about 24%, about 24.4%, about 24.5%, about 24.9%, about 25%, about 25.4%, about 25.5%, about 25.9%, about 26%, about 26.4%, about 26.5%, about 26.9%, about 27%, about 27.4%, about 27.5%, about 27.9%, about 28%, about 28.4%, about 28.5%, about 28.9%, about 29%, about 29.4%, about 29.5%, about 29.9%, about 30%, about 30.4%, about 30.5%, about 30.9%, about 31%, about 31.4%, about 31.5%, about 31.9%, about 32%, about 32.4%, about 32.5%, about 32.9%, about 33%, about 33.4%, about 33.5%, about 33.9%, about 34%, about 34.4%, about 34.5%, about 34.9%, about 35%, about 35.4%, about 35.5%, about 35.9%, about 36%, about 36.4%, about 36.5%, about 36.9%, about 37%, about 37.4%, about 37.5%, about 37.9%, about 38%, about 38.4%, about 38.5%, about 38.9%, about 40%, about 40.4%, about 40.5%, about 40.9%, about 41%, about 41.4%, about 41.5%, about 41.9%, about 42%, about 42.4%, about 42.5%, about 42.9%, about 43%, about 43.4%, about 43.5%, about 43.9%, about 44%, about 44.4%, about 44/5%, about 44.9%, about 45%, about 45.4%, about 45.5%, about 45.9%, about 46%, about 46.4%, about 46.5%, about 46.9%, about 47%, about 47.4%, about 47.5%, about 47.9%, about 48%, about 48.4%, about 48.5%, about 48.9%, about 49%, about 49.4%, about 49.5%, about 49.9%, about 50%, about 50.4%, about 50.5%, about 50.9%, about 51%, about 51.4%, about 51.5%, about 51.9%, about 52%, about 52.4%, about 52.5%, about 52.9%, about 53%, about 53.4%, about 53.5%, about 53.9%, about 54%, about 54.4%, about 54.5%, about 54.9%, about 55%, about 55.4%, about 55.5%, about 55.9%, about 56%, about 56.4%, about 56.5%, about 56.9%, about 57%, about 57.4%, about 57.5%, about 57.9%, about 58%, about 58.4%, about 58.5%, about 58.9%, about 59%, about 59.4%, about 59.5%, about 59.9%, about 60%, about 60.4%, about 60.5%, about 60.9%, about 61%, about 61.4%, about 61.5%, about 61.9%, about 62%, about 62.4%, about 62.5%, about 62.9%, about 63%, about 63.4%, about 63.5%, about 63.9%, about 64%, about 64.4%, about 64.5%, about 64.9%, about 65%, about 65.4%, about 65.5%, about 65.9%, about 66%, about 66.4%, about 66.5%, about 66.9%, about 67%, about 67.4%, about 67.5%, about 67.9%, about 68%, about 68.4%, about 68.5%, about 68.9%, about 69%, about 69.4%, about 69.5%, or about 69.9% of the total lipid molar concentration.

22. The lipid nanoparticle composition of any one of claims 1-21, wherein the total molar concentration of said one or more lipid conjugates is from about 0.01% to about 10%, from about 0.1% to about 10%, from about 0.2% to about 8%, from about 0.5% to about 5%, from about 0.1% to about 2.5%, from about 0.1% to about 2%, from about 0.1% to about 1.5%, or from about 1% to about 2% of the total lipid molar concentration.

23. The lipid nanoparticle composition of any one of claims 1-22, wherein the total molar concentration of said one or more lipid conjugates is about 0.1%, about 0.5%, about 1%, about 1.5%, about 2%, or about 2.5% of the total lipid molar concentration.

24. The lipid nanoparticle composition of any one of claims 16-23, wherein said one or more non-cationic lipids comprise said one or more phospholipids and said one or more steroids.

25. The lipid nanoparticle composition of any one of claims 16-24, wherein said lipid nanoparticle comprises said one or more cationic lipids, said one or more phospholipids, said one or more steroids, and said one or more lipid conjugates at a molar ratio set forth in any lipid nanoparticle composition disclosed in FIG. 1.

26. The lipid nanoparticle composition of any one of claims 16-25, wherein said lipid nanoparticles comprise:
(a) said one or more cationic lipids at a total molar concentration of about 40% of the total lipid molar concentration;
(b) said one or more steroids at a total molar concentration of about 48.5% of the total lipid molar concentration;
(c) said one or more phospholipids at a total molar concentration of about 10% of the total lipid molar concentration; and
(d) said one or more lipid conjugates at a molar concentration of about 1.5% of the total lipid molar concentration.

27. The lipid nanoparticle composition of any one of claims 16-25, wherein said lipid nanoparticles comprise:
(a) the one or more cationic lipids at a total molar concentration of about 50% of the total lipid molar concentration;
(b) the one or more steroids at a total molar concentration of about 38.5% of the total lipid molar concentration;
(c) the one or more phospholipids at a total molar concentration of about 10% of the total lipid molar concentration; and
(d) the one or more lipid conjugates at a total molar concentration of about 1.5% of the total lipid molar concentration.

28. The lipid nanoparticle composition of any one of claims 1-27, wherein said one or more cationic lipids includes DLin-DMA, DLin-MC3-DMA, DLin-KC2-DMA, DODMA, SS-OP, SS-M, SS-E, SS-EC, SS-LC, SS-OC, DOTAP, DOTMA, DODAP, DOGS, DOSPA, DC-Chol, GL-67, BGTC, DDAB, DORIE, DMRIE, GAP-DLRIE, diC14-amidine, L319, C12-200, OF-02, TT3, ZA3-Ep10, or a derivative thereof.

29. The lipid nanoparticle composition of any one of claims 1-28, wherein said one or more cationic lipids includes DLin-MC3-DMA.

30. The lipid nanoparticle composition of any one of claims 2-29, wherein said one or more cationic lipids includes at least one chemically modified cationic lipid bound to said apolipoprotein, and at least one unmodified cationic lipid, wherein the molar ratio of said chemically modified cationic lipid to said unmodified cationic lipid is between about 1:1 and about 1:300, between about 1:10 and about 1:200, between about 1:25 and about 1:175, between about 1:50 and about 1:150, between about 1:100 and about 1:150, or between about 1:125 and about 1:150.

31. The lipid nanoparticle composition of any one of claims 2-30, wherein the molar ratio of said chemically modified cationic lipid to said unmodified cationic lipid is about 1:50, about 1:60, about 1:70, about 1:80, about 1:90, about 1:100, about 1:105, about 1:110, about 1:115, 1:120, about 1:125, 1:130, about 1:135, 1:140, about 1:145, about 1:149, or about 1:150.

32. The lipid nanoparticle composition of any one of claims 2-31, wherein said one or more non-cationic lipids includes at least one chemically modified non-cationic lipid bound to the apolipoprotein, and at least one unmodified non-cationic lipid, wherein the molar ratio of said chemically modified non-cationic lipid to said non-cationic cationic lipid is between about 1:1 and about 1:300, between about 1:10 and about 1:200 between about 1:25 and about 1:175, between about 1:50 and about 1:150, between about 1:100 and about 1:150, or between about 1:125 and about 1:150.

33. The lipid nanoparticle composition of any one of claims 2-32, wherein the molar ratio of said chemically modified non-cationic lipid to said unmodified non-cationic lipid is about 1:50, about 1:60, about 1:70, about 1:80, about 1:90, about 1:100, about 1:105, about 1:110, about 1:115, 1:120, about 1:125, 1:130, about 1:135, 1:140, about 1:145, about 1:149, or about 1:150.

34. The lipid nanoparticle composition of any one of claims 16-33, wherein said one or more phospholipids includes DSPC, DOPE, POPC, DDPC, DEPA-NA, DEPC, DEPE, DEPG-NA, DLOPC, DLPA-NA, DLPC, DLPE, DLPG-NA, DLPG-NH4, DLPS-NA, DMPA-NA, DMPC, DMPE, DMPG-NA, DMPG-NH4, DMPG-NH4/NA, DOPA, DOPG, DOPS, DPPA, DPPC, DPPE, DPPG, DPPS, DSPA, DSPG, DSPS, EPC, HEPC, MPPC, MSPC, PMPC, POPC, POPE, POPG, PSPC, SMPC, SOPC, SPPC, or a derivative thereof.

35. The lipid nanoparticle composition of any one of claims 16-34, wherein said one or more phospholipids includes DSPC.

36. The lipid nanoparticle composition of any one of claims 16-35, wherein said one or more phospholipids includes at least one chemically modified phospholipid bound to the apolipoprotein, and at least one unmodified phospholipid, wherein the molar ratio of said chemically modified phospholipid to said unmodified phospholipid is between about 1:1 and about 1:300, between about 1:10 and about 1:200, between about 1:25 and about 1:175, between about 1:50 and about 1:150, between about 1:100 and about 1:150, or between about 1:125 and about 1:150.

37. The lipid nanoparticle composition of claim 36, wherein the molar ratio of said chemically modified phospholipid to said unmodified phospholipid is about 1:50, about 1:60, about 1:70, about 1:80, about 1:90, about 1:100, about 1:105, about 1:110, about 1:115, 1:120, about 1:125, 1:130, about 1:135, 1:140, about 1:145, about 1:149, or about 1:150.

38. The lipid nanoparticle composition of any one of claims 19-37, wherein said one or more steroids includes cholesterol, ergosterol, hopanoids, hydroxysteroid, phytosterol, zoosterol, gonane, testosterone, cholic acid, dexamethasone, lanosterol, progesterone, medrogestone, beta-sitosterol, cholestane, cholanes, pregnanes, androstanes, estranes, or a derivative thereof.

39. The lipid nanoparticle composition of any one of claims 19-38, wherein said one or more steroids includes cholesterol.

40. The lipid nanoparticle composition of any one of claims 19-39, wherein said one or more steroids includes at least one chemically modified steroid bound to the apolipoprotein, and at least one unmodified steroid, wherein the molar ratio of said chemically modified steroid to said unmodified steroid is between about 1:1 and about 1:300, between about 1:10 and about 1:200, between about 1:25 and about 1:175, between about 1:50 and about 1:150, between about 1:100 and about 1:150, or between about 1:125 and about 1:150.

41. The lipid nanoparticle composition of claim 40, wherein the molar ratio of said chemically modified steroid to said unmodified steroid is about 1:50, about 1:60, about 1:70, about 1:80, about 1:90, about 1:100, about 1:105, about 1:110, about 1:115, 1:120, about 1:125, 1:130, about 1:135, 1:140, about 1:145, about 1:149, or about 1:150.

42. The lipid nanoparticle composition of any one of claims 1-41, wherein said one or more lipid conjugates includes DMG-PEG550, DMG-PEG1000, DMG-PEG2000, DMG-PEG5000, DSPE-PEG550, DSPE-PEG1000, DSPE-PEG2000, DSPE-PEG5000, DMPE-PEG550, DMPE-PEG1000, DMPE-PEG2000, DMPE-PEG5000, C18-PEG5000, C18-PEG2000, C18-PEG1000, C18-PEG550, C16-PEG5000, C16-PEG2000, C16-PEG1000, C16-PEG550, C14-PEG5000, C14-PEG2000, C14-PEG1000, C14-PEG550, C12-PEG5000, C12-PEG2000, C12-PEG1000, C12-PEG550, cholesterol-PEG5000, cholesterol-PEG2000, cholesterol-PEG1000, cholesterol-PEG550, or a derivative thereof.

43. The lipid nanoparticle composition of any one of claims 1-42, wherein said one or more lipid conjugates includes DMG-PEG2000, DMG-PEG5000, or DSPE-PEG2000.

44. The lipid nanoparticle composition of any one of claims 2-43, wherein said one or more lipid conjugates includes at least one chemically modified lipid conjugate bound to the apolipoprotein, and at least one unmodified lipid conjugate, wherein the molar ratio of said chemically modified lipid conjugate to said unmodified lipid conjugate is between about 1:1 and about 1:300, between about 1:10 and about 1:200, between about 1:25 and about 1:175, between about 1:50 and about 1:150, between about 1:100 and about 1:150, or between about 1:125 and about 1:150.

45. The lipid nanoparticle composition of claim 44, wherein the molar ratio of said chemically modified lipid conjugate to said unmodified lipid conjugate is about 1:50, about 1:60, about 1:70, about 1:80, about 1:90, about 1:100, about 1:105, about 1:110, about 1:115, 1:120, about 1:125, 1:130, about 1:135, 1:140, about 1:145, about 1:149, or about 1:150.

46. The lipid nanoparticle composition of any one of claims 2-45, wherein said one or more lipid conjugates includes a chemically modified DSPE-PEG bound to said apolipoprotein, and an unmodified DMG-PEG.

47. The lipid nanoparticle composition of any one of claims 2-45, wherein said one or more lipid conjugates includes a chemically modified DSPE-PEG2000 bound to the apolipoprotein, and an unmodified DMG-PEG2000.

48. The lipid nanoparticle composition of any one of claims 19-47, wherein said one or more cationic lipids is DLin-MC3-DMA, said one or more phospholipids is DSPC, said one or more steroids is cholesterol, and said one or more lipid conjugates are pegylated lipids, wherein said pegylated lipids include unmodified DMG-PEG2000 and a chemically modified DSPE-PEG2000, comprising an amino group, that is bound to said apolipoprotein, wherein said apolipoprotein is ApoE.

49. The lipid nanoparticle composition of any one of claims 19-47, wherein said one or more cationic lipids is DLin-MC3-DMA, said one or more phospholipids is DSPC, said one or more steroids is cholesterol, and said one or more lipid conjugates are pegylated lipids, wherein said pegylated lipids include unmodified DMG-PEG2000 and a chemically modified DSPE-PEG2000, comprising a carboxy group, that is bound to said apolipoprotein, wherein said apolipoprotein is ApoE.

50. The lipid nanoparticle composition of any one of claims 19-47, wherein said one or more cationic lipids is DLin-MC3-DMA, said one or more phospholipids is DSPC, said one or more steroids is cholesterol, and said one or more lipid conjugates are pegylated lipids, wherein said pegylated lipids include unmodified DMG-PEG2000 and a chemically modified DSPE-PEG2000, comprising a maleimide group, that is bound to said apolipoprotein, wherein said apolipoprotein is ApoE.

51. The lipid nanoparticle composition of any one of claims 19-47, wherein the molar ratio of DLin-MC3-DMA, DPSC, cholesterol, and said one or more pegylated lipids is about 40:10:48.5:1.5, wherein said one or more pegylated lipids includes a chemically modified DSPE-PEG2000 bound to ApoE and unmodified DMG-PEG2000, wherein the molar ratio of said chemically modified DSPE-PEG2000 to said unmodified DMG-PEG2000 is about 1:149.

52. The lipid nanoparticle composition of any one of claims 19-47, wherein the molar ratio of DLin-MC3-DMA, DPSC, cholesterol, and said one or more pegylated lipids is about 50:10:38.5:1.5, wherein said one or more pegylated lipids includes a chemically modified DSPE-PEG2000 bound to ApoE and unmodified DMG-PEG2000, wherein the molar ratio of said chemically modified DSPE-PEG2000 to said unmodified DMG-PEG2000 is about 1:149.

53. The lipid nanoparticle composition of any one of claims 1-52, wherein said lipid nanoparticles do not comprise a biological targeting molecule having specificity for a cell surface antigen on a target cell.

54. The lipid nanoparticle composition of claim 53, wherein said biological targeting molecule is an antibody, or antigen-binding fragment thereof.

55. The lipid nanoparticle composition of claim 53 or 54, wherein said cell surface antigen is present on a human immune cell.

56. The lipid nanoparticle composition of claim 55, wherein said human immune cell is a human T cell, a human natural killer cell, a human B cell, or a human macrophage.

57. The lipid nanoparticle composition of claim 53 or 54, wherein said cell surface antigen is present on a human induced pluripotent stem cell (iPSC).

58. The lipid nanoparticle composition of any one of claims 1-57, wherein said lipid nanoparticles comprise a nucleic acid.

59. The lipid nanoparticle composition of claim 58, wherein said lipid nanoparticles comprise a cationic charge:phosphate ratio between said one or more cationic lipids and said nucleic acid of from about 1 to about 20, from about 2 to about 16, from about 4 to about 12, or from about 6 to about 10.

60. The lipid nanoparticle composition of claim 58 or 59, wherein said lipid nanoparticles comprise a cationic charge:phosphate ratio between said one or more cationic lipids and said nucleic acid of about 8.

61. The lipid nanoparticle composition of any one of claims 58-60, wherein said nucleic acid is an mRNA.

62. The lipid nanoparticle composition of any one of claims 58-60, wherein said nucleic acid is a DNA molecule.

63. The lipid nanoparticle composition of claim 62, wherein said DNA molecule is a double-stranded DNA molecule (dsDNA).

64. The lipid nanoparticle composition of claim 62 or 63, wherein said DNA molecule is a recombinant DNA construct.

65. The lipid nanoparticle composition of any one of claims 58-60, wherein said nucleic acid is an RNA interference (RNAi) molecule.

66. The lipid nanoparticle composition of any one of 58-64, wherein said nucleic acid comprises a nucleic acid sequence encoding an engineered nuclease.

67. The lipid nanoparticle composition of claim 66, wherein said engineered nuclease is an engineered meganuclease, a CRISPR system nuclease, a TALEN, a compact TALEN, a zinc finger nuclease, or a megaTAL.

68. The lipid nanoparticle composition of claim 66 or 67, wherein said engineered nuclease is an engineered meganuclease.

69. The lipid nanoparticle composition of any one of claims 66-68, wherein said engineered nuclease has specificity for a recognition sequence within a T cell receptor (TCR) alpha gene, a TCR alpha constant region (TRAC) gene, a TCR beta gene, or a TCR beta constant region (TRBC) gene.

70. The lipid nanoparticle composition of any one of claims 66-69, wherein said engineered nuclease is an engineered meganuclease having specificity for a recognition sequence comprising SEQ ID NO: 2.

71. The lipid nanoparticle composition of claim 62-64, wherein said nucleic acid comprises a donor template.

72. The lipid nanoparticle composition of claim 71, wherein said donor template comprises a 5′ homology arm and a 3′ homology arm.

73. The lipid nanoparticle composition of claim 71 or 72, wherein said donor template comprises a nucleic acid sequence encoding a polypeptide of interest.

74. The lipid nanoparticle composition of claim 73, wherein said polypeptide of interest is a chimeric antigen receptor (CAR) or an exogenous TCR.

75. The lipid nanoparticle composition of claim 65, wherein said RNAi molecule is a short hairpin RNA (shRNA), a small interfering RNA (siRNA), a hairpin siRNA, a microRNA (miRNA), a precursor miRNA, or an miRNA-adapted shRNA.

76. A population of eukaryotic cells comprising said lipid nanoparticle composition of any one of claims 1-75.

77. The population of claim 76, wherein said eukaryotic cells are human immune cells.

78. The population of claim 77, wherein said human immune cells are human T cells, human NK cells, human B cells, or human macrophages.

79. The population of claim 76, wherein said eukaryotic cells are human iPSCs.

80. A method for transfecting a population of eukaryotic cells, said method comprising contacting said population of eukaryotic cells with said lipid nanoparticle composition of any one of claims 1-75.

81. The method of claim 80, wherein said eukaryotic cells are human immune cells.

82. The method of claim 81, wherein said human immune cells are human T cells, human NK cells, human B cells, or human macrophages.

83. The method of claim 80, wherein said eukaryotic cells are human iPSCs.

84. A method for introducing a nucleic acid into a population of eukaryotic cells, said method comprising contacting said population of eukaryotic cells with a lipid nanoparticle composition of any one of claims 1-75, wherein said lipid nanoparticles comprise said nucleic acid.

85. The method of claim 84, wherein said lipid nanoparticles comprise a cationic charge:phosphate ratio between said one or more cationic lipids and said nucleic acid of from about 1 to about 20, from about 2 to about 16, from about 4 to about 12, or from about 6 to about 10.

86. The method of claim 84 or 85, wherein said lipid nanoparticles comprise a cationic charge:phosphate ratio between said one or more cationic lipids and said nucleic acid of about 8.

87. The method of any one of claims 84-86, wherein said nucleic acid is an mRNA.

88. The method of any one of claims 84-86, wherein said nucleic acid is a DNA molecule.

89. The method of claim 88, wherein said DNA molecule is a double-stranded DNA molecule (dsDNA).

90. The method of claim 88 or 89, wherein said DNA molecule is a recombinant DNA construct.

91. The method of any one of claims 84-86, wherein said nucleic acid is an RNA interference (RNAi) molecule.

92. The method of claim 87, wherein said nucleic acid molecule is an mRNA, wherein said mRNA encodes an engineered nuclease, wherein said engineered nuclease is expressed in said eukaryotic cells, and wherein said engineered nuclease binds and cleaves a recognition sequence in the genome of said eukaryotic cells to generate a cleavage site.

93. The method of any one of claims 88-90, wherein said nucleic acid molecule is a DNA molecule, wherein said DNA molecule comprises a nucleic acid sequence encoding an engineered nuclease, wherein said engineered nuclease is expressed in said eukaryotic cells, and wherein said engineered nuclease binds and cleaves a recognition sequence in the genome of said eukaryotic cells to generate a cleavage site.

94. The method of claim 92 or 93, wherein said recognition sequence is within a target gene.

95. The method of claim 84, wherein expression of a polypeptide encoded by said target gene is disrupted by non-homologous end joining at said cleavage site.

96. The method of any one of claims 92-94, wherein said method further comprises introducing a second nucleic acid into said population of eukaryotic cells, wherein said second nucleic acid comprises a donor template.

97. The method of claim 96, wherein said donor template is inserted into said cleavage site by homologous recombination.

98. The method of claim 97, wherein expression of a polypeptide encoded by said target gene is disrupted by insertion of said donor template into said cleavage site.

99. The method of any one of claims 96-98, wherein said donor template is flanked by a 5′ homology arm and a 3′ homology arm having homology to sequences 5′ upstream and 3′ downstream, respectively, of said cleavage site.

100. The method of any one of claims 96-99, wherein said donor template is introduced into said population of eukaryotic cells within 48 hours after said eukaryotic cells are contacted with said lipid nanoparticles comprising said nucleic acid encoding said engineered nuclease.

101. The method of any one of claims 96-99, wherein said donor template is introduced into said population of eukaryotic cells between 0-24 hours after said eukaryotic cells are contacted with said lipid nanoparticles comprising said nucleic acid encoding said engineered nuclease.

102. The method of any one of claims 96-99, wherein said donor template is introduced into said population of eukaryotic cells between 24-48 hours after said eukaryotic cells are contacted with said lipid nanoparticles comprising said nucleic acid encoding said engineered nuclease.

103. The method of any one of claims 96-99, wherein said donor template is introduced into said population of eukaryotic cells within 12 hours after said eukaryotic cells are contacted with said lipid nanoparticles comprising said nucleic acid encoding said engineered nuclease.

104. The method of any one of claims 96-103, wherein said donor template is introduced into said population of eukaryotic cells by a recombinant virus.

105. The method of claim 104, wherein said recombinant virus is a recombinant adeno-associated virus (AAV).

106. The method of any one of claims 96-103, wherein said donor template is introduced into said population of eukaryotic cells by a second lipid nanoparticle composition.

107. The method of claim 106, wherein said donor template is comprised by a recombinant DNA construct encapsulated by said second lipid nanoparticle composition.

108. The method of claim 106 or 107, wherein said second lipid nanoparticle composition comprises said lipid nanoparticle composition of any one of claims 1-75.

109. The method of any one of claims 96-108, wherein said donor template comprises a nucleic acid sequence encoding a polypeptide of interest.

110. The method of claim 109, wherein said polypeptide of interest is a CAR or an exogenous TCR.

111. The method of any one of claims 94-110, wherein said target gene is a TCR alpha gene, a TRAC gene, a TCR beta gene, or a TRBC gene.

112. The method of any one of claims 94-111, wherein said target gene is a TRAC gene.

113. The method of any one of claims 92-112, wherein said engineered nuclease is an engineered meganuclease, a CRISPR system nuclease, a TALEN, a compact TALEN, a zinc finger nuclease, or a megaTAL.

114. The method of any one of claims 92-113, wherein said engineered nuclease is an engineered meganuclease.

115. The method of any one of claims 92-114, wherein said engineered nuclease has specificity for a recognition sequence within a TCR alpha gene, a TRAC gene, a TCR beta gene, or a TRBC gene.

116. The method of any one of claims 92-115, wherein said engineered nuclease is an engineered meganuclease having specificity for a recognition sequence comprising SEQ ID NO: 2.

117. The method of any one of claims 96-116, wherein said donor template is inserted into the genome of said eukaryotic cells between positions 13 and 14 of SEQ ID NO: 2.

118. The method of any one of claims 88-90, wherein said nucleic acid is a DNA molecule, and wherein said DNA molecule comprises a donor template.

119. The method of claim 118, wherein said DNA molecule is a dsDNA.

120. The method of claim 118 or 119, wherein said DNA molecule is a recombinant DNA construct.

121. The method of any one of claims 118-120, wherein said donor template comprises a nucleic acid sequence encoding a polypeptide of interest.

122. The method of claim 121, wherein said polypeptide of interest is a CAR or an exogenous TCR.

123. The method of any one of claims 118-122, wherein said method further comprises introducing a second nucleic acid into said population of eukaryotic cells, wherein said second nucleic acid encodes an engineered nuclease, wherein said engineered nuclease is expressed in said eukaryotic cells, and wherein said engineered nuclease binds and cleaves a recognition sequence in the genome of said eukaryotic cells to generate a cleavage site.

124. The method of claim 123, wherein said second nucleic acid encoding said engineered nuclease is an mRNA.

125. The method of claim 124, wherein said mRNA is introduced using a lipid nanoparticle composition.

126. The method of claim 124, wherein said mRNA is introduced by electroporation.

127. The method of claim 123, wherein said second nucleic acid encoding said engineered nuclease is introduced into said eukaryotic cells using a recombinant virus.

128. The method of claim 127, wherein said recombinant virus is a recombinant AAV.

129. The method of claim 123, wherein said second nucleic acid is a recombinant DNA construct comprising a nucleic acid sequence encoding said engineered nuclease.

130. The method of claim 129, wherein said recombinant DNA construct is introduced by transfection.

131. The method of claim 129, wherein said recombinant DNA construct is introduced using a lipid nanoparticle composition.

132. The method of claim 131, wherein said lipid nanoparticle composition is said lipid nanoparticle composition of any one of claims 1-73.

133. The method of any one of claims 123-132, wherein said cleavage site is within a target gene.

134. The method of any one of claims 123-133, wherein said donor template is inserted into said cleavage site by homologous recombination.

135. The method of any one of claims 123-134, wherein said donor template is flanked by a 5′ homology arm and a 3′ homology arm having homology to sequences 5′ upstream and 3′ downstream, respectively, of said cleavage site.

136. The method of any one of claims 133-135, wherein insertion of said donor template into said cleavage site disrupts expression of a polypeptide encoded by said target gene.

137. The method of any one of claims 133-136, wherein said target gene is a TCR alpha gene, a TRAC gene, a TCR beta gene, or a TRBC gene.

138. The method of any one of claims 133-137, wherein said target gene is a TRAC gene.

139. The method of any one of claims 123-138, wherein said donor template is introduced into said population of eukaryotic cells within 48 hours after introduction of said nucleic acid encoding said engineered nuclease.

140. The method of any one of claims 123-138, wherein said donor template is introduced into said population of eukaryotic cells between 0-24 hours after introduction of said nucleic acid encoding said engineered nuclease.

141. The method of any one of claims 123-138, wherein said donor template is introduced into said population of eukaryotic cells between 24-48 hours after introduction of said nucleic acid encoding said engineered nuclease.

142. The method of any one of claims 123-138, wherein said donor template is introduced into said population of eukaryotic cells within 12 hours after introduction of said nucleic acid encoding said engineered nuclease.

143. The method of any one of claims 123-142, wherein said engineered nuclease is an engineered meganuclease, a CRISPR system nuclease, a TALEN, a compact TALEN, zinc finger nuclease, or a megaTAL.

144. The method of any one of claims 123-143, wherein said engineered nuclease is an engineered meganuclease.

145. The method of any one of claims 123-144, wherein said engineered nuclease has specificity for a recognition sequence within a TCR alpha gene, a TRAC gene, a TCR beta gene, or a TRBC gene.

146. The method of any one of claims 123-145, wherein said engineered nuclease is an engineered meganuclease having specificity for a recognition sequence comprising SEQ ID NO: 2.

147. The method of any one of claims 123-146, wherein said donor template is inserted into the genome of said eukaryotic cells between positions 13 and 14 of SEQ ID NO: 2.

148. The method of claim 91, wherein said RNAi molecule is an shRNA, an siRNA, a hairpin siRNA, an miRNA, a precursor miRNA, or a microRNA-adapted shRNA.

149. The method of claim 91 or 148, wherein said RNAi molecule is inhibitory against beta-2 microglobulin.

150. The method of any one of claim 91, 148, or 149, wherein said eukaryotic cells are human immune cells.

151. The method of claim 150, wherein said human immune cells are human T cell, human NK cells, human B cells, or human macrophages.

152. The method of any one of claim 91, 148, or 149, wherein said eukaryotic cells are iPSCs.

153. A population of eukaryotic cells prepared by the method of any one of claims 80-152.

154. A pharmaceutical composition comprising a pharmaceutically-acceptable carrier and said population of eukaryotic cells of claim 153.

155. A method for reducing the number of target cells in a subject in need thereof, said method comprising administering a therapeutically effective amount of a pharmaceutical composition of claim 154, wherein said population of eukaryotic cells express a CAR or an exogenous TCR, and wherein said CAR or said exogenous TCR has specificity for an antigen present on said target cells.

156. The method of claim 155, wherein said eukaryotic cells are human T cells, human NK cells, human B cells, or human macrophages.

157. The method of claim 155 or 156, wherein said method is a method of immunotherapy.

158. The method of any one of claims 155-157, wherein said target cells are cancer cells.

159. The method of any one of claims 155-158, wherein said method reduces the size of said cancer.

160. The method of any one of claims 155-159, wherein said method eradicates the cancer in said subject.

161. A kit for transfecting a population of eukaryotic cells comprising a lipid nanoparticle composition of any one of claims 1-75.

162. The kit of claim 161, wherein said kit further comprises a reagent that enhances the transfection efficiency of said lipid nanoparticle composition.


SEQUENCE LISTING (4 sequences)

SEQ ID NO: 1 - protein, 163 aa, Chlamydomonas reinhardtii
MNTKYNKEFLLYLAGFVDGDGSIIAQIKPNQSYKFKHQLSLAFQVTQKTQRRWFLDKLVDEIGVGYVRDRGSVSDYILSEIKPLHNFLTQLQPFLKLKQKQANLVLKIIWRLPSAKESPDKFLEVCTWVDQIAALNDSKTRKTTSETVRAVLDSLSEKKKSSP

SEQ ID NO: 2 - DNA, 22 bp, Homo sapiens
tggcctggagcaacaaatctga

SEQ ID NO: 3 - DNA, 22 bp, Homo sapiens
accggacctcgttgtttagact

SEQ ID NO: 4 - protein, 354 aa, Artificial Sequence
  Synthesized
MNTKYNKEFLLYLAGFVDGDGSIYAVIYPHQRAKFKHFLKLLFTVSQSTKRRWFLDKLVDEIGVGYVYDLPRTSEYRLSEIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSIYACIRPRQGSKFKHRLTLGFAVGQKTQRRWFLDKLVDEIGVGYVYDRGSVSEYVLSEIKPLHNFLTQLQPFLKLKQKQANLVLKIIEQLPSAKESPDKFLEVCTWVDQIAALNDSKTRKTTSETVRAVLDSLSEKKKSSP